SEQUENCE LISTING (22 sequences)

SEQ ID NO: 1 - protein, 125 aa, synthetic construct
EVQLVESGGGLVQPGGSLRLSCAASGFNIYYNYIHWVRQAPGKGLEWVAEFSPYSGSTYYADSVKGRFTISADTSKNTAYLQMNSLRAEDTAVYYCARSAAIVDWYDYFKGFDYWGQGTLVTVSS

SEQ ID NO: 2 - protein, 107 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQYGSYLYTFGQGTKVEIK

SEQ ID NO: 3 - protein, 125 aa, synthetic construct
EVQLVESGGGLVQPGGSLRLSCAASGFNIYYNYIHWVRQAPGKGLEWVAEFSPYSGSTYYADSVKGRFTISADTSKNTAYLQMNSLRAEDTAVYYCARSAAIKDWYDYFKGFDYWGQGTLVTVSS

SEQ ID NO: 4 - protein, 125 aa, synthetic construct
EVQLVESGGGLVQPGGSLRLSCAASGFNIYYNYIHWVRQAPGKGLEWVAEFSPYSGSTYYADSVKGRFTISADTSKNTAYLQMNSLRAEDTAVYYCARSAAIVDFYDYFKGFDYWGQGTLVTVSS

SEQ ID NO: 5 - protein, 125 aa, synthetic construct
EVQLVESGGGLVQPGGSLRLSCAASGFNIYYNYIHWVRQAPGKGLEWVAEFSPYSGSTYYADSVKGRFTISADTSKNTAYLQMNSLRAEDTAVYYCARSAAIVDVYDYFKGFDYWGQGTLVTVSS

SEQ ID NO: 6 - protein, 125 aa, synthetic construct
EVQLVESGGGLVQPGGSLRLSCAASGFNIYYNYIHWVRQAPGKGLEWVAEFSPYSGSTYYADSVKGRFTISADTSKNTAYLQMNSLRAEDTAVYYCARSAAIVDLYDYFKGFDYWGQGTLVTVSS

SEQ ID NO: 7 - protein, 125 aa, synthetic construct
EVQLVESGGGLVQPGGSLRLSCAASGFNIYYNYIHWVRQAPGKGLEWVAEFSPYSGSTYYADSVKGRFTISADTSKNTAYLQMNSLRAEDTAVYYCARSAAIVDAYDYFKGFDYWGQGTLVTVSS

SEQ ID NO: 8 - protein, 125 aa, synthetic construct
EVQLVESGGGLVQPGGSLRLSCAASGFNIYYNYIHWVRQAPGKGLEWVAEFSPYSGSTYYADSVKGRFTISADTSKNTAYLQMNSLRAEDTAVYYCARSAAIVDWVDYFKGFDYWGQGTLVTVSS

SEQ ID NO: 9 - protein, 125 aa, synthetic construct
EVQLVESGGGLVQPGGSLRLSCAASGFNIYYNYIHWVRQAPGKGLEWVAEFSPYSGSTYYADSVKGRFTISADTSKNTAYLQMNSLRAEDTAVYYCARSAAIVDWADYFKGFDYWGQGTLVTVSS

SEQ ID NO: 10 - protein, 125 aa, synthetic construct
EVQLVESGGGLVQPGGSLRLSCAASGFNIYYNYIHWVRQAPGKGLEWVAEFSPYSGSTYYADSVKGRFTISADTSKNTAYLQMNSLRAEDTAVYYCARSAAIVDWHDYFKGFDYWGQGTLVTVSS

SEQ ID NO: 11 - protein, 125 aa, synthetic construct
EVQLVESGGGLVQPGGSLRLSCAASGFNIYYNYIHWVRQAPGKGLEWVAEFSPYSGSTYYADSVKGRFTISADTSKNTAYLQMNSLRAEDTAVYYCARSAAIVDWKDYFKGFDYWGQGTLVTVSS

SEQ ID NO: 12 - protein, 125 aa, synthetic construct
EVQLVESGGGLVQPGGSLRLSCAASGFNIHYNYIHWVRQAPGKGLEWVAEFSPYSGSTYYADSVKGRFTISADTSKNTAYLQMNSLRAEDTAVYYCARSAAIVDWYDYFKGFDYWGQGTLVTVSS

SEQ ID NO: 13 - protein, 125 aa, synthetic construct
EVQLVESGGGLVQPGGSLRLSCAASGFNIYHNYIHWVRQAPGKGLEWVAEFSPYSGSTYYADSVKGRFTISADTSKNTAYLQMNSLRAEDTAVYYCARSAAIVDWYDYFKGFDYWGQGTLVTVSS

SEQ ID NO: 14 - protein, 125 aa, synthetic construct
EVQLVESGGGLVQPGGSLRLSCAASGFNIYYNYIHWVRQAPGKGLEWVAEFSPYRGSTYYADSVKGRFTISADTSKNTAYLQMNSLRAEDTAVYYCARSAAIVDWYDYFKGFDYWGQGTLVTVSS

SEQ ID NO: 15 - protein, 125 aa, synthetic construct
EVQLVESGGGLVQPGGSLRLSCAASGFNIYYNYIHWVRQAPGKGLEWVAEFSPYSGRTYYADSVKGRFTISADTSKNTAYLQMNSLRAEDTAVYYCARSAAIVDWYDYFKGFDYWGQGTLVTVSS

SEQ ID NO: 16 - protein, 125 aa, synthetic construct
EVQLVESGGGLVQPGGSLRLSCAASGFNIYYNYIHWVRQAPGKGLEWVAEFSPYSGSTYYADSVKGRFTISADTSKNTAYLQMNSLRAEDTAVYYCARSAAIVDWQDYFKGFDYWGQGTLVTVSS

SEQ ID NO: 17 - protein, 125 aa, synthetic construct
EVQLVESGGGLVQPGGSLRLSCAASGFNIYYNYIHWVRQAPGKGLEWVAEFSPYSGSTYYADSVKGRFTISADTSKNTAYLQMNSLRAEDTAVYYCARSAAIVDWRDYFKGFDYWGQGTLVTVSS

SEQ ID NO: 18 - protein, 107 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQYGSKLYTFGQGTKVEIK

SEQ ID NO: 19 - protein, 125 aa, synthetic construct
EVQLVESGGGLVQPGGSLRLSCAASGFNIYYNYIHWVRQAPGKGLEWVAEFSPYSGSTYYADSVKGRFTISADTSKNTAYLQMNSLRAEDTAVYYCARSAAIVDHYDYFKGFDYWGQGTLVTVSS

SEQ ID NO: 20 - protein, 125 aa, synthetic construct
EVQLVESGGGLVQPGGSLRLSCAASGFNIYYNYIHWVRQAPGKGLEWVAEFSPYSGSTYYADSVKGRFTISADTSKNTAYLQMNSLRAEDTAVYYCARSAAIVDKYDYFKGFDYWGQGTLVTVSS

SEQ ID NO: 21 - protein, 125 aa, synthetic construct
EVQLVESGGGLVQPGGSLRLSCAASGFNIYYNYIHWVRQAPGKGLEWVAEFSPYSGSTYYADSVKGRFTISADTSKNTAYLQMNSLRAEDTAVYYCARSAAIVDRYDYFKGFDYWGQGTLVTVSS

SEQ ID NO: 22 - protein, 125 aa, synthetic construct; identical to SEQ ID NO: 10

(19) United States
(12) Patent Application Publication
(10) Pub. No.: US 2026/0317437 A1
(43) Pub. Date: Oct. 15, 2026

(54) METHODS AND COMPOSITIONS FOR REDUCING ANTIBODY VISCOSITY

(71) Applicant: Genentech, Inc., South San Francisco, CA (US)

(72) Inventors: Paul John CARTER, Hillsborough, CA (US); Jing DAI, Foster City, CA (US); Saeed IZADI, Walnut Creek, CA (US)

(73) Assignee: Genentech, Inc., South San Francisco, CA (US)

(21) Appl. No.: 19/483,219

(22) PCT Filed: May 10, 2024
(86) PCT No.: PCT/US2024/028793

§ 371 Date: Nov. 10, 2025

Related U.S. Application Data
(60) Provisional application No. 63/466,219, filed on May 12, 2023.
(60) Provisional application No. 63/587,649, filed on Oct. 3, 2023.

(51) Int. Cl. C07K 16/28; C40B 40/10; G01N 11/00; G01N 33/68

(52) U.S. Cl. CPC C07K 16/2869; G01N 11/00; G01N 33/6854; C07K 2317/55; C07K 2317/565; C07K 2317/92; C40B 40/10; G01N 2011/008

(57) ABSTRACT

Provided herein are methods of identifying an antigen binding polypeptide variant (e.g., an antibody variant, an antigen-binding fragment thereof, or a variant of an antibody-based construct) having reduced viscosity relative to a parental antigen binding polypeptide (e.g., an antibody, an antigen-binding fragment thereof, or an antibody construct). Also provided are antigen binding polypeptide variants produced by the methods. Further provided are related libraries and methods of screening such libraries. Also provided are methods of identifying one or more amino acids in an antibody, an antigen binding fragment of an antibody, or an antibody construct that affect the viscosity of the antibody, the antigen binding fragment, or the antibody construct. Also provided are exemplary antibody variable domains with reduced viscosity.

### CROSS-REFERENCE TO RELATED APPLICATIONS

This application claims priority to, and the benefit of, U.S. Provisional Patent Application Nos. 63/466,219, filed on May 12, 2023, and 63/587,649, filed on Oct. 3, 2023, the contents of each of which are hereby incorporated herein by reference in their entirety.

### FIELD OF THE INVENTION

The present application relates to methods of decreasing the viscosity of antibody compositions, as well as antibodies produced by the methods.

### SEQUENCE LISTING

The content of the electronic sequence listing (146392066640seglist.xml; Size: 22,642 bytes; and Date of Creation: May 8, 2024) is hereby incorporated herein by reference in its entirety.

### BACKGROUND OF THE INVENTION

Over the last three decades, the United States Food and Drug Administration (US FDA) has approved approximately 125 biologic drugs, including antibody-based therapeutics, e.g., monoclonal antibodies (mAbs), antibody-drug conjugates, and Fc-fusion proteins. The majority of antibody-based therapeutics are administered intravenously (IV). However, IV administration typically requires visits to a healthcare facility and is usually associated with lengthy administration times, higher medical costs, and lower patient compliance. An alternative route, namely, subcutaneous administration, is being increasingly used for patients with chronic diseases, as frequent dosing of biologic drugs may be needed over patients' lifetimes. Presently, approximately 30% of approved antibody therapeutics are given by subcutaneous injection. See, e.g., Carter and Rajpal (2022) Cell, 185(15): P2789-2805. Subcutaneous injections can sometimes be self-administered by patients using ready-to-use delivery devices, which are advantageous for comfort and privacy. Concentrated solutions of antibody-based therapeutics contained in such devices may provide the additional benefit of longer intervals between injections, which can reduce healthcare costs by minimizing hospital or clinic visits and increasing patient compliance and adherence to a treatment regimen.

The development of formulations comprising antibody-based therapeutics for subcutaneous administration is a critical consideration. The volume of drug that can be injected subcutaneously is typically 1-2 mL, although volumes up to 3 mL are possible. Such low volumes necessitate that antibody-based therapeutics be formulated, e.g, at concentrations typically ≥150 mg/mL to deliver the required dose. However, such highly concentrated solutions can, in some cases, demonstrate high viscosities, leading to complications in the development of antibody-based therapeutics as drug products suitable for subcutaneous administration High viscosity formulations can increase the injection time and may cause pain at the site of injection, adversely affecting patient compliance. Additionally, high viscosity formulations also pose difficulties during the manufacture of the antibody-based therapeutic.

Although factors contributing to the viscosity of high concentration antibody solutions have been extensively studied, predicting the viscosity of an antibody, antigen binding fragment thereof, or antibody-based construct remains a challenge. What is needed in the art are improved methods of predicting and decreasing the viscosity of compositions comprising high concentrations of antibody-based therapeutics, including antibodies, antigen binding fragments thereof, and antibody-based constructs.

### SUMMARY OF THE INVENTION

In some embodiments, provided is a method of identifying an antigen binding polypeptide variant having reduced viscosity relative to a parental antigen binding polypeptide, comprising: (a) identifying one or more wild type amino acids in the parental antigen binding polypeptide that have high spatial aggregation propensity (SAP) values and/or high solvent-accessible surface area (SASA) ratios; (b) substituting the one or more wild type amino acids identified as having the high SAP values and/or the high SASA ratios with amino acids having lower SAP values and/or lower SASA ratios as compared to the wild type amino acids: (c) measuring the viscosity of the antigen binding polypeptide variant; and (d) identifying the antigen binding polypeptide variant having reduced viscosity relative to parental polypeptide. In some embodiments, the one or more wild type amino acids in the parental antigen binding polypeptide are surface exposed. In some embodiments, the one or more wild type amino acid(s) are aromatic amino acids, and the one or more aromatic amino acids are substituted with charged amino acids or amino acids having smaller side chains. In some embodiments, the one or more aromatic amino acids are selected from the group consisting of: W, Y, and F. In some embodiments, the charged amino acid(s) or amino acid(s) having smaller side chains are selected from the group consisting of A, I, L, V, D, E, H, K, R, N, Q, S, and T. In some embodiments, the one or more wild type amino acid(s) have smaller side chains, and wherein the one or more amino acids that have smaller chains are substituted with charged amino acids. In some embodiments, the one or more wild type amino acid(s) have smaller side chains are selected from the group consisting of: A, I, L, V, N, Q, S, G, P, C, M, or T. In some embodiments, the charged amino acid(s) are selected from the group consisting of: R, K, H, D, and E.

In some embodiments, the high SAP values are 2 or greater, such as 2.3 or greater. In some embodiments, the high SASA ratios are 0.5 or greater. In some embodiments, the high SASA ratio(s) are 0.25 or greater. In some embodiments, the viscosity of the antigen binding polypeptide variant is assessed via dynamic light scattering (DLS). In some embodiments, the viscosity of the antigen binding polypeptide variant is measured via cone and plate rheometry. In some embodiments, the viscosity of the antigen binding polypeptide variant is measured at a high concentration. In some embodiments, the high concentration is between about 50 mg/mil and 300 mg/ml.

In some embodiments, the parental antigen binding polypeptide is an antibody, an antigen-binding fragment of an antibody, or an antibody construct, the antibody or antibody construct comprises one or more of a CDR-H1, a CDR-H2, a CDR-H3, a CDR-L1, a CDR-L2 and a CDR-L3, and one or more wild type amino acids in the one or more CDR-H1, CDR-H2, CDR-H3, CDR-L1, CDR-L2 and CDR-L3 are identified as having high SAP values and/or high SASA ratios. In some embodiments, the parental antigen binding polypeptide is an antibody. In some embodiments, the antibody is a therapeutic antibody. In some embodiments, the antibody is a chimeric antibody, a humanized antibody, or a human antibody. In some embodiments, the antibody is a monospecific antibody, a bispecific antibody, or a multispecific antibody. In some embodiments, the antibody comprises a human IgG Fc region. In some embodiments, the human IgG constant region is an IgG1, IgG2, or IgG4 Fc region. In some embodiments, the parental polypeptide is an antigen binding fragment of an antibody. In some embodiments, the antigen binding fragment is a Fab, a F(ab′)2, a trispecific Fab₃, an scFv, a monovalent IgG, a diabody, a triabody, an scFv-Vc, a minibody, a V_{H}H, a V-NAR, an hcIgG, or an IgNAR. In some embodiments, the parental polypeptide is an antibody construct. In some embodiments, the antibody construct is a CrossMab, a dual action Fab (DAF), a DVD-IgG, or a knob-in-hole bispecific antibody.

In some embodiments, a method provided herein comprises the step of subjecting the antigen binding polypeptide variant to at least one affinity maturation step. In some embodiments the one or more substituted amino acids in the antigen binding polypeptide variant that were introduced to replace the one or more wild type amino acids having high SAP values and/or high SASA ratios are not randomized during affinity maturation. In some embodiments, provided is a polypeptide variant produced by a method described herein.

In some embodiments, provided is a library comprising a plurality of antigen binding polypeptide variants, wherein at least one variant comprises one or more amino acid substitutions relative to a parental antigen binding polypeptide, wherein the one or more amino acid substitutions relative to the parental antigen binding polypeptide replace one or more wild type amino acids identified as having the high SAP values and the high SASA ratios with amino acids having lower SAP values and/or lower SASA ratios as compared to the wild type amino acids. In some embodiments the plurality comprises at least 1,000 unique variants. In some embodiments, the plurality of antigen binding variants is a plurality of antibodies, antigen-binding fragments of antibodies, or antibody constructs.

Also provide herein is a method of predicting one or more amino acids in an antigen binding polypeptide that affect the viscosity of the antigen binding polypeptide, the method comprising: identifying one or more amino acids in the antigen binding polypeptide that have high spatial aggregation propensity (SAP) values and/or high solvent-accessible surface area (SASA) ratios, wherein the one or more amino acids that have high SAP values and/or high SASA ratios are predicted to affect viscosity of the antigen binding polypeptide. Also provided herein is a method of predicting one or more amino acids in an antibody, an antigen binding fragment of an antibody, or an antibody construct that affect the viscosity of the antibody, the antigen binding fragment, or the antibody construct, wherein the antibody, the antigen binding fragment, or the antibody construct comprises one or more of a CDR-H1, a CDR-H2, a CDR-H3, a CDR-L1, a CDR-L2 and a CDR-L3, the method comprising: identifying one or more amino acid positions in the one or more of the CDR-H1, the CDR-H2, the CDR-H3, the CDR-L1, the CDR-L2 and the CDR-L3 that have high spatial aggregation propensity (SAP) values and high solvent-accessible surface area (SASA) ratios, wherein the one or more amino acid positions that have the SAP values and the high SASA ratios are predicted to affect viscosity of the antibody, the antigen binding fragment, or the antibody construct.

In some embodiments, provided is an antibody heavy chain variable domain (V_{H}) set forth in any one of SEQ ID NOs:3-17 or 19-22. In some embodiments, provided is an antibody comprising a heavy chain variable domain (V_{H}) set forth in any one of SEQ ID NOs:3-17 or 19-22 and a light chain variable domain (V_{L}) set forth in SEQ ID NO:2.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTIONS OF THE DRAWINGS

FIG. 1 provides the results of experiments that were performed to measure the viscosity of alanine substitution variants of an anti-GCGR IgG1 antibody comprising a V_{H} set forth in SEQ ID NO:1 and a V_{L} set forth in SEQ ID NO:2. See, Tilegenova et al. (2020) mAbs, 12(1): 1692764.

FIG. 2 provides surface electrostatic potential map of an anti-GCGR Fab fragment comprising a V_{H} set forth in SEQ ID NO:1 and a V_{L} set forth in SEQ ID NO:2 (left) and the spatial aggregation propensity (SAP) of the Fab (right). On the left, the surface charge patch was analyzed based on the electrostatic potential map calculated by Adaptive Poisson-Boltzmann Solver (APBS) on an averaged structure derived from molecular dynamics (MD) trajectories. On the right, the circles indicate large hydrophobic patches that overlap with four CDRS. The representation is averaged SAP value calculated for each atom across all representation MD trajectories.

FIG. 3 shows 6 CDRS including 4 CDRS (L3, H1, H2, and H3) having multiple (but not all) residues with high-SAP values (e.g., having a SAP value of ≥2, such as 2.3 or greater) for the anti-GCGR Fab whose SAP is shown in the right side of FIG. 2.

FIG. 4 shows the aromatic amino acid residues in the CDRs of the anti-GCGR Fab that were substituted in Example 1A.

FIG. 5 shows a viscosity analysis of anti-GCGR IgG1 variants targeting aromatic residues with high SAP scores. Specifically, individual alanine mutations were introduced for each of the 10 parent aromatic residues with high SAP scores. The viscosity of these single mutants, together with a negative control mutant (VH Y79A, were measured at a concentration of 180 mg/mL by rheometry at 25.0° C. in 20 mM histidine acetate, pH 5.5 (bars, left axis). The SAP score for the selected sites is also shown (right axis). Additionally, three double mutants combining two¬ viscosity-reducing mutations were tested to evaluate any additive effects. The viscosity of the parent anti-GCGR antibody is shown as a reference with a dotted line.

FIG. 6 shows a comparison of the viscosity of single substitution variant IgG1 antibodies shown in FIG. 5 relative to the parent IgG1 antibody vs. SAP value of each original wild type amino acid.

FIG. 7 shows a selection of the anti-GCGR viscosity hotspot sites combining high SAP score and high SASA ratio. The SAP score and SASA ratio were calculated and plotted against each other for all sites in the anti-GCGR Fab fragment. The dotted lines indicate cutoff values used for high SASA ratio (0.25) and high SAP score (2.3). Sites with both high SASA ratio and high SAP scores (upper right quadrant) were selected for mutational analysis to evaluate their contribution to high viscosity. Additionally, representative sites in the high SASA ratio and low SAP region (labeled residues in the upper left quadrant) as well as in the low SASA ratio and high SAP region (labeled residues in the lower right quadrant) were also included to provide a more comprehensive analysis.

FIG. 8 shows a plot of DLS interaction parameter (k_{D}, ml/g) and viscosity (η, cP) for exemplary anti-GCGR IgG1 antibody variants. The Pearson's correlation efficient between the two sets of measurements was calculated as −0.73. The solid thick black line represents the Pearson linear regression fit. The DLS interaction parameter was measured between 2-10 mg/mL. Viscosity was measured at 180 mg/mL.

FIG. 9 provides a table reporting screening of DLS interaction parameters for ~200 anti-GCGR IgG1 variants. Shown are DLS interaction parameters measured at 25.0° C. in 20 mM histidine acetate, pH 5.5. Variants with exceptionally low diffusion coefficient are highlighted (grey), indicating their polydispersed behavior in solution. The parental residues are highlighted using (P). Mutants with expression or purification issues or exhibited significant aggregation are also highlighted (black).

FIG. 10 shows a plot of DLS interaction parameter (k_{D}, ml/g) for each the anti-GCGR IgG1 antibody variant tested in FIG. 9 and each variant's dissociation constant (K_{D}, M). The K_{D} of the anti-GCGR mutants to the extracellular domain of GCGR was determined by SPR. Variants with an interaction parameter of >12 mL/g and binding affinities within 5-fold of the parent antibody are noted in the upper left quadrant. The parent antibody is also highlighted using a label.

### DETAILED DESCRIPTION OF THE INVENTION

### Overview

Provided herein are methods of identifying antigen binding polypeptide variants (e.g., antibodies, antigen-binding fragments thereof, and antibody-based constructs) having reduced viscosities relative to a parental antigen binding polypeptide. Also provided are antigen binding polypeptide variants (e.g., antibodies, antigen-binding fragments thereof, and antibody-based constructs) produced by the methods. Libraries comprising pluralities of antigen binding polypeptide variants having reduced viscosity are also described herein. Additionally, the present application provides methods of predicting one or more amino acids in an antibody, an antigen binding fragment of an antibody, or an antibody construct that affect the viscosity of the antibody, the antigen binding fragment, or the antibody construct.

The present application is based on Applicant's unexpected finding that key viscosity-affecting amino acids in an antigen binding polypeptide (e.g., an antibody, an antigen-binding fragment thereof, or an antibody-based construct) are characterized by having high spatial aggregation propensity (SAP) values (e.g., SAP values ≥2, such as 2.3 or greater) and/or high solvent accessible surface area (SASA) ratios (e.g., SASA ratios ≥0.25, for example ≥0.5). Applicant also unexpectedly found that substituting one or more amino acids in the parental antigen binding polypeptide that have been identified as having high SAP values and/or high SASA ratios with amino acid(s) having lower SAP values and/or lower SASA ratios produces antigen binding polypeptide variants with reduced viscosity. The inventors also unexpectedly found that there is a strong negative correlation between the dynamic light scattering (DLS) interaction parameter and viscosity, highlighting the potential of using DLS as a predictive assay independently or to complement other activity assays in early-stage drug discovery. DLS-screening methods require less protein content than certain other viscosity assays, such as cone-and-plate rheometry. Moreover, DLS-screening methods can be used alone or in conjunction with binding assays to identifying binding proteins with a desired viscosity and/or target binding affinity to further enable the therapeutic development of binding proteins, such as antibodies.

Thus, provided herein in some aspects is a method of identifying an antigen binding polypeptide variant having reduced viscosity relative to a parental antigen binding polypeptide, the method comprising: (a) identifying one or more wild type amino acids in the parental antigen binding polypeptide that have high spatial aggregation propensity (SAP) values and/or high solvent-accessible surface area (SASA) ratios; (b) substituting the one or more wild type amino acids identified as having the high SAP values and/or the high SASA ratios with amino acids having lower SAP values and/or lower SASA ratios as compared to the wild type amino acids; and -(c) identifying the antigen binding polypeptide variant having reduced viscosity relative to parental polypeptide. In some embodiments, the method further comprises determining, such as by measuring, the viscosity of the antigen binding polypeptide variant.

In certain aspects, provided herein is a method of identifying an antigen binding polypeptide variant having reduced viscosity relative to a parental antigen binding polypeptide, the method comprising: (a) identifying one or more wild type amino acids in the parental antigen binding polypeptide that have high spatial aggregation propensity (SAP) values and/or high solvent-accessible surface area (SASA) ratios; (b) substituting the one or more wild type amino acids identified as having the high SAP values and/or the high SASA ratios with amino acids having lower SAP values and/or lower SASA ratios as compared to the wild type amino acids; (c) determining, such as by measuring, the viscosity of the antigen binding polypeptide variant, and (d) identifying the antigen binding polypeptide variant having reduced viscosity relative to parental polypeptide.

As described herein, in some embodiments, aspects of the methods may be performed using one or more processors, e.g., using a computer. Thus, in some embodiments, polypeptides and polypeptide sequences, e.g., antigen binding polypeptide variants, may refer to various forms based on the context such as a molecular structure or data representing a sequence.

Unless defined otherwise herein, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 3D Ed., John Wiley and Sons, New York (2006) provides one of skill with a general dictionary of many of the terms used in this invention. Although any methods and materials similar or equivalent to those described herein can be used in the practice or testing of the present invention, the preferred methods and materials are described. Numeric ranges are inclusive of the numbers defining the range. Unless otherwise indicated, nucleic acids are written left to right in 5′ to 3′ orientation; amino acid sequences are written left to right in amino to carboxy orientation, respectively. Practitioners are particularly directed to Sambrook et al., 1989, and Ausubel F M et al., 1993, for definitions and terms of the art. It is to be understood that this invention is not limited to the particular methodology, protocols, and reagents described, as these may vary.

Numeric ranges are inclusive of the numbers defining the range.

Unless otherwise indicated, nucleic acids are written left to right in 5′ to 3′ orientation; amino acid sequences are written left to right in amino to carboxy orientation, respectively.

The headings provided herein are not limitations of the various aspects or embodiments which can be had by reference to the specification as a whole. Accordingly, the terms defined immediately below are more fully defined by reference to the specification as a whole.

### Definitions

Before describing the embodiments in detail, it is to be understood that the present disclosure is not limited to particular compositions or biological systems, which can, of course, vary. It is also to be understood that the terminology used herein is for the purpose of describing particular embodiments only, and is not intended to be limiting.

As used in this specification and the appended claims, the singular forms “a,” “an,” and “the” include plural referents unless the content clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the present disclosure include “comprising,” “consisting,” and “consisting essentially of” aspects and embodiments.

As used herein, the term “antibody” is used in the broadest sense and specifically covers intact antibodies (e.g., full length antibodies), antibody fragments (including without limitation Fab, F(ab′)2, Fab′-SH, Fv, diabodies, scFv, scFv-Fc, single domain antibodies, single heavy chain antibodies, and single light chain antibodies), monoclonal antibodies, and polyclonal antibodies, so long as they exhibit the desired biological activity (e.g., epitope binding). “Antibodies” (or “Abs”) and “immunoglobulins” (or “Igs”) are glycoproteins having the same structural characteristics. While antibodies exhibit binding specificity to a specific antigen, immunoglobulins include both antibodies and other antibody-like molecules which lack antigen specificity. Polypeptides of the latter kind are, for example, produced at low levels by the lymph system and at increased levels by myelomas.

As a frame of reference, as used herein an immunoglobulin will refer to the structure of an immunoglobulin G (IgG). However, one skilled in the art would understand/recognize that an antibody of any immunoglobulin class may be utilized in the inventive method described herein. For clarity, an IgG molecule contains a pair of heavy chains (HCs) and a pair of light chains (LCs). Each LC has one variable domain (V_{L}) and one constant domain (C_{L}), while each HC has one variable (V_{H}) and three constant domains (C_{H}1, C_{H}2, and C_{H}3). The C_{H}1 and C_{H}2 domains are connected by a hinge region. This structure is well known in the art.

Briefly, the basic 4-chain antibody unit is a heterotetrameric glycoprotein composed of two light (L) chains and two heavy (H) chains (an IgM antibody consists of 5 of the basic heterotetramer unit along with an additional polypeptide called J chain, and therefore contain 10 antigen binding sites, while secreted IgA antibodies can polymerize to form polyvalent assemblages comprising 2-5 of the basic 4-chain units along with J chain). In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (V_{H}) followed by three constant domains (C_{H}) for each of the α and γ chains and four C_{H} domains for μ and ε isotypes. Each L chain has at the N-terminus, a variable domain (V_{L}) followed by a constant domain (C_{L}) at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, CT, 1994, page 71 and Chapter 6.

The L chain from any vertebrate species can be assigned to one of two clearly distinct types, called kappa and lambda, based on the amino acid sequences of their constant domains. Depending on the amino acid sequence of the constant domain of their heavy chains (C_{H}), immunoglobulins can be assigned to different classes or isotypes. There are five classes of immunoglobulins: IgA, IgD, IgE, IgG, and IgM, having heavy chains designated α, δ, γ, ε, and μ, respectively. The γ and α classes are further divided into subclasses on the basis of relatively minor differences in C_{H} sequence and function, e.g., humans express the following subclasses: IgG1, IgG2, IgG3, IgG4, IgA1, and IgA2.

As used herein, the term “isolated” antibody may refer to an antibody that is substantially free of other cellular material. In one embodiment, an isolated antibody is substantially free of other proteins from the same species. In another embodiment, an isolated antibody is expressed by a cell from a different species and is substantially free of other proteins from the different species. In some embodiments, an “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. An antibody may be rendered substantially free of naturally associated components (or components associated with the cellular expression system used to produce the antibody) by isolation, using protein purification techniques well known in the art. In some embodiments, the antibody will be purified (1) to greater than 75% by weight of antibody as determined by the Lowry method, and most preferably more than 80%, 90%, 95% or 99% by weight, or (2) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

As used herein, the term “epitope” means any antigenic determinant on an antigen to which the paratope of an antibody binds. Epitopic determinants usually consist of chemically active surface groupings of molecules such as amino acids or sugar side chains and usually have specific three-dimensional structural characteristics, as well as specific charge characteristics.

As used herein, the term “native antibodies and immunoglobulins” are usually heterotetrameric glycoproteins of about 150,000 daltons, composed of two identical light (L) chains and two identical heavy (H) chains. Each light chain is linked to a heavy chain by one covalent disulfide bond (also termed a “VH/VL pair”), while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light- and heavy-chain variable domains. See, e.g., Chothia et al., J. Mol. Biol., 186:651 (1985); Novotny and Haber, Proc. Natl. Acad. Sci. U.S.A., 82:4592 (1985).

As used herein, the term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework (FR). The variable domains of native heavy and light chains each comprise four FR regions, largely adopting a R-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen-binding site of antibodies. See, e.g., Kabat et al., Sequences of Proteins of Immunological Interest, Fifth Edition, National Institute of Health, Bethesda, Md. (1991). The constant domains are not involved directly in binding an antibody to an antigen, but exhibit various effector functions, such as participation of the antibody in antibody-dependent cellular toxicity. Variable region sequences of interest include the humanized variable region sequences for CD47 antibodies described in detail elsewhere herein.

The term “hypervariable region (HVR)” or “complementarity determining region (CDR)” may refer to the subregions of the VH and VL domains characterized by enhanced sequence variability and/or formation of defined loops. These include three CDRs in the VH domain (H1, H2, and H3) and three CDRs in the VL domain (L1, L2, and L3). H3 is believed to be critical in imparting fine binding specificity, with L3 and H3 showing the highest level of diversity. See Johnson and Wu, in Methods in Molecular Biology 248:1-25 (Lo, ed., Human Press, Totowa, N.J., 2003).

A number of CDR/HVR delineations are known. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. The residues from each of these HVRs/CDRs are noted below. “Framework” or “FR” residues are those variable domain residues other than the HVR/CDR residues.

| Loop | Kabat | AbM | Chothia | Contact | |
|---|---|---|---|---|---|
| L1 | L24-L34 | L24-L34 | L26-L32 | L30-L36 | |
| L2 | L50-L56 | L50-L56 | L50-L52 | L46-L55 | |
| L3 | L89-L97 | L89-L97 | L91-L96 | L89-L96 | |
| H1 | H31-H35B | H26-H35B | H26-H32 | H30-H35B | (Kabat |
| | | | | | Numbering) |
| H1 | H31-H35 | H26-H35 | H26-H32 | H30-H35 | (Chothia |
| | | | | | Numbering) |
| H2 | H50-H65 | H50-H58 | H53-H55 | H47-H58 | |
| H3 | H95-H102 | H95-H102 | H96-H101 | H93-H101 | |

“Extended” HVRs are also known: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the VH (Kabat numbering).

“Numbering according to Kabat” may refer to the numbering system used for heavy chain variable domains or light chain variable domains of the compilation of antibodies in Kabat et al., supra. The actual linear amino acid sequence may contain fewer or additional amino acids corresponding to a shortening of, or insertion into, a FR or HVR of the variable domain. The Kabat numbering of residues may be determined for a given antibody by alignment at regions of homology of the sequence of the antibody with a “standard” Kabat numbered sequence. Typically, the Kabat numbering is used when referring to a residue in the variable domains (approximately residues 1-107 of the light chain and residues 1-113 of the heavy chain), whereas the EU numbering system or index (e.g., the EU index as in Kabat, numbering according to EU IgG1) is generally used when referring to a residue in the heavy chain constant region.

As used herein, a “monoclonal” antibody refers to an antibody obtained from a population of substantially homogeneous antibodies, e.g., substantially identical but allowing for minor levels of background mutations and/or modifications. “Monoclonal” denotes the substantially homogeneous character of antibodies, and does not require production of the antibody by any particular method. In some embodiments, a monoclonal antibody is selected by its HVR, VH, and/or VL sequences and/or binding properties, e.g., selected from a pool of clones (e.g., recombinant, hybridoma, or phage-derived). A monoclonal antibody may be engineered to include one or more mutations, e.g., to affect binding affinity or other properties of the antibody, create a humanized or chimeric antibody, improve antibody production and/or homogeneity, engineer a multispecific antibody, resultant antibodies of which are still considered to be monoclonal in nature. A population of monoclonal antibodies may be distinguished from polyclonal antibodies as the individual monoclonal antibodies of the population recognize the same antigenic site. A variety of techniques for production of monoclonal antibodies are known; see, e.g., the hybridoma method (e.g., Köhler and Milstein, Nature, 256:495-97 (1975); Hongo et al., Hybridoma, 14 (3): 253-260 (1995), Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and U.S. Pat. No. 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995).

The term “Fc region,” as used herein, generally refers to a dimer complex comprising the C-terminal polypeptide sequences of an immunoglobulin heavy chain, wherein a C-terminal polypeptide sequence is that which is obtainable by papain digestion of an intact antibody. The Fc region may comprise native or variant Fc sequences. Although the boundaries of the Fc sequence of an immunoglobulin heavy chain might vary, the human IgG heavy chain Fc sequence comprises about position Cys226, or from about position Pro230, to the carboxyl terminus of the Fc sequence. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991. The Fc sequence of an immunoglobulin generally comprises two constant domains, a C_{H}2 domain and a C_{H}3 domain, and optionally comprises a C_{H}4 domain. By “Fc polypeptide” herein is meant one of the polypeptides that make up an Fc region, e.g., a monomeric Fc. An Fc polypeptide may be obtained from any suitable immunoglobulin, such as human IgG1, IgG2, IgG3, or IgG4 subtypes, IgA, IgE, IgD or IgM. An Fc polypeptide may be obtained from mouse, e.g., a mouse IgG2a. The Fc region comprises the carboxy-terminal portions of both H chains held together by disulfides. The effector functions of antibodies are determined by sequences in the Fc region; this region is also the part recognized by Fc receptors (FcR) found on certain types of cells. In some embodiments, an Fc polypeptide comprises part or all of a wild type hinge sequence (generally at its N terminus). In some embodiments, an Fc polypeptide does not comprise a functional or wild type hinge sequence.

“Fc component” as used herein refers to a hinge region, a C_{H}2 domain or a C_{H}3 domain of an Fc region.

In certain embodiments, the Fc region comprises an IgG Fc region, preferably derived from a wild-type human IgG Fc region. In certain embodiments, the Fc region is derived from a “wild type” mouse IgG, such as a mouse IgG2a. By “wild-type” human IgG Fc or “wild type” mouse IgG Fc it is meant a sequence of amino acids that occurs naturally within the human population or mouse population, respectively. Of course, just as the Fc sequence may vary slightly between individuals, one or more alterations may be made to a wild type sequence and still remain within the scope of the invention. For example, the Fc region may contain alterations such as a mutation in a glycosylation site or inclusion of an unnatural amino acid.

“Chimeric” antibodies may refer to an antibody with one portion of the heavy and/or light chain from a particular isotype, class, or organism and another portion from another isotype, class, or organism. In some embodiments, the variable region will be from one source or organism, and the constant region will be from another.

“Humanized antibodies” may refer to antibodies with predominantly human sequence and a minimal amount of non-human (e.g., mouse or chicken) sequence. In some embodiments, a humanized antibody has one or more HVR sequences (bearing a binding specificity of interest) from an antibody derived from a non-human (e.g., mouse or chicken) organism grafted onto a human recipient antibody framework (FR). In some embodiments, non-human residues are further grafted onto the human framework (not present in either source or recipient antibodies), e.g., to improve antibody properties. In general, a humanized antibody will comprise substantially all of at least one, and typically two, variable domains, in which all or substantially all of the hypervariable loops correspond to those of a non-human immunoglobulin, and all or substantially all of the FRs are those of a human immunoglobulin sequence. The humanized antibody optionally will also comprise at least a portion of an immunoglobulin constant region (Fc), typically that of a human immunoglobulin. See Jones et al., Nature 321:522-525 (1986); Riechmann et al., Nature 332:323-329 (1988); and Presta, Curr. Op. Struct. Biol. 2:593-596 (1992).

A “human” antibody may refer to an antibody having an amino acid sequence which corresponds to that of an antibody produced by a human and/or has been made using any of the techniques for making human antibodies as disclosed herein. Human antibodies can be produced using various techniques known in the art, including phage-display libraries. Hoogenboom and Winter, J. Mol. Biol., 227:381 (1991); Marks et al., J. Mol. Biol., 222:581 (1991); preparation of human monoclonal antibodies as described in Cole et al., Monoclonal Antibodies and Cancer Therapy, Alan R. Liss, p. 77 (1985); Boerner et al., J. Immunol., 147(1):86-95 (1991); and by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology) or chickens with human immunoglobulin sequence(s) (see, e.g., WO2012162422, WO2011019844, and WO2013059159).

There are five major classes of immunoglobulins: IgA, IgD, IgE, IgG, and IgM, and several of these can be further divided into subclasses (isotypes), e.g., IgG1, IgG2, IgG3, IgG4, IgA1, IgA2. The heavy-chain constant domains that correspond to the different classes of immunoglobulins are called α, δ, ε, γ, and μ, respectively. The subunit structures and three-dimensional configurations of different classes of immunoglobulins are well known.

As used herein, the term “antibody fragment,” and all grammatical variants thereof, are defined as a portion of an intact antibody comprising the antigen binding site or variable region of the intact antibody which, in certain instances, is free of the constant heavy chain domains (i.e. CH2, CH3, and/or CH4, depending on antibody isotype) of the Fc region of the intact antibody. Examples of antibody fragments include Fab, Fab′, Fab′-SH, F(ab′)₂, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv (scFv) molecules, (2) single chain polypeptides containing only one light chain variable domain, or a fragment thereof that contains the three CDRs of the light chain variable domain, without an associated heavy chain moiety, and (3) single chain polypeptides containing only one heavy chain variable region, or a fragment thereof containing the three CDRs of the heavy chain variable region, without an associated light chain moiety; and multi-specific or multivalent structures formed from antibody fragments. In an antibody fragment comprising one or more heavy chains, the heavy chain(s) can contain any constant domain sequence (e.g. CH1 in the IgG isotype) found in a non-Fc region of an intact antibody, and/or can contain any hinge region sequence found in an intact antibody, and/or can contain a leucine zipper sequence fused to or situated in the hinge region sequence or the constant domain sequence of the heavy chain(s).

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen. “Fv” is the minimum antibody fragment which contains a complete antigen-recognition and -binding site. In a two-chain Fv species, this region consists of a dimer of one heavy- and one light-chain variable domain in tight, non-covalent association. In a single-chain Fv species (scFv), one heavy- and one light-chain variable domain can be covalently linked by a flexible peptide linker such that the light and heavy chains can associate in a “dimeric” structure analogous to that in a two-chain Fv species. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site. See, e.g., Plückthun, in The Pharmacology of Monoclonal Antibodies, Vol. 113, Rosenburg and Moore eds., Springer-Verlag, New York, pp. 269-315 (1994).

The Fab fragment also contains the constant domain of the light chain and the first constant domain (CH₁) of the heavy chain. Fab′ fragments differ from Fab fragments by the addition of a few residues at the carboxy terminus of the heavy chain CH₁ domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

As used herein, the term “monoclonal antibody” (mAb) refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Each mAb is directed against a single determinant on the antigen. In addition to their specificity, the monoclonal antibodies are advantageous in that they can be synthesized by hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made in an immortalized B cell or hybridoma thereof, or may be made by recombinant DNA methods.

The monoclonal antibodies include hybrid and recombinant antibodies produced by splicing a variable (including hypervariable) domain of an CD47 antibody with a constant domain (e.g. “humanized” antibodies), or a light chain with a heavy chain, or a chain from one species with a chain from another species, or fusions with heterologous proteins, regardless of species of origin or immunoglobulin class or subclass designation, as well as antibody fragments (e.g., Fab, F(ab′)₂, and Fv), so long as they exhibit the desired biological activity.

The monoclonal antibodies herein specifically include chimeric antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity.

An antibody (such as a monospecific or multispecific antibody) “which binds an antigen of interest” is one that binds the antigen, e.g., a protein, with sufficient affinity such that the antibody is useful as a diagnostic and/or therapeutic agent in targeting a protein or a cell or tissue expressing the protein, and does not significantly cross-react with other proteins. In such embodiments, the extent of binding of the antibody to a “non-target” protein will be less than about 10% of the binding of the antibody to its particular target protein as determined by fluorescence activated cell sorting (FACS) analysis or radioimmunoprecipitation (RIA) or ELISA. With regard to the binding of antibody to a target molecule, the term “specific binding” or “specifically binds to” or is “specific for” a particular polypeptide or an epitope on a particular polypeptide target means binding that is measurably different from a nonspecific interaction (e.g., a non-specific interaction may be binding to bovine serum albumin or casein). Specific binding can be measured, for example, by determining binding of a molecule compared to binding of a control molecule. For example, specific binding can be determined by competition with a control molecule that is similar to the target, for example, an excess of non-labeled target. In this case, specific binding is indicated if the binding of the labeled target to a probe is competitively inhibited by excess unlabeled target. The term “specific binding” or “specifically binds to” or is “specific for” a particular polypeptide or an epitope on a particular polypeptide target as used herein can be exhibited, for example, by a molecule having a K_{D} for the target of at least about 200 nM, alternatively at least about 150 nM, alternatively at least about 100 nM, alternatively at least about 60 nM, alternatively at least about 50 nM, alternatively at least about 40 nM, alternatively at least about 30 nM, alternatively at least about 20 nM, alternatively at least about 10 nM, alternatively at least about 8 nM, alternatively at least about 6 nM, alternatively at least about 4 nM, alternatively at least about 2 nM, alternatively at least about 1 nM, or greater affinity. In one embodiment, the term “specific binding” refers to binding where a multispecific antibody binds to a particular polypeptide or epitope on a particular polypeptide without substantially binding to any other polypeptide or polypeptide epitope.

Commercially available reagents referred to in the Examples were used according to manufacturer's instructions unless otherwise indicated. The source of those cells identified in the following Examples, and throughout the specification, by ATCC accession numbers is the American Type Culture Collection, Manassas, VA. Unless otherwise noted, the present invention uses standard procedures of recombinant DNA technology, such as those described hereinabove and in the following textbooks: Sambrook et al., supra; Ausubel et al., Current Protocols in Molecular Biology (Green Publishing Associates and Wiley Interscience, NY, 1989); Innis et al., PCR Protocols: A Guide to Methods and Applications (Academic Press, Inc., NY, 1990); Harlow et al., Antibodies: A Laboratory Manual (Cold Spring Harbor Press, Cold Spring Harbor, 1988); Gait, Oligonucleotide Synthesis (IRL Press, Oxford, 1984); Freshney, Animal Cell Culture, 1987; Coligan et al., Current Protocols in Immunology, 1991.

All references cited herein, including patent applications and publications, are hereby incorporated by reference in their entirety.

### Methods of Identifying Antigen Binding Polypeptide Variants Having Reduced Viscosity Relative to a Parental Antigen Binding Polypeptide

In some embodiments, provided is a method of identifying an antigen binding polypeptide variant (e.g., an antibody variant, an antigen binding fragment of an antibody variant, or a variant of an antibody construct) having reduced viscosity relative to a parental antigen binding polypeptide (e.g., an antibody, an antigen binding fragment thereof, or an antibody construct), the method comprising: (a) identifying one or more wild type amino acids in the parental antigen binding polypeptide that have high spatial aggregation propensity (SAP) values and/or high solvent-accessible surface area (SASA) ratios; (b) substituting the one or more wild type amino acids identified as having high SAP values and/or high SASA ratios with amino acids having lower SAP values and/or lower SASA ratios as compared to the wild type amino acids; (c) measuring the viscosity of the antigen binding polypeptide variant; and (d) identifying the antigen binding polypeptide variant having reduced viscosity relative to the parental antigen binding polypeptide. In some embodiments, the method comprises (a) identifying one or more wild type amino acids in the parental antigen binding polypeptide that have high SAP values and high SASA ratios. In some embodiments, the method comprises the step of (b) substituting the one or more wild type amino acids identified as having high SAP values and high SASA ratios with amino acids having lower SAP values and lower SASA ratios as compared to the wild type amino acids. In some embodiments, the method comprises determining the SAP values and/or SASA ratios of the one or more substituted amino acids in the antigen binding polypeptide variant after step (b) and prior to step (c).

In some embodiments, provided is a method of reducing viscosity of an antigen binding polypeptide (e.g., an antibody, an antigen binding fragment thereof, or an antibody construct), method comprising: (a) identifying one or more wild type amino acids in the antigen binding polypeptide that have high spatial aggregation propensity (SAP) values and/or high solvent-accessible surface area (SASA) ratios; (b) substituting the one or more wild type amino acids identified as having high SAP values and/or high SASA ratios with amino acids having lower SAP values and/or lower SASA ratios as compared to the wild type amino acids to produce an antigen binding polypeptide variant (e.g., an antibody variant, an antigen binding fragment of an antibody variant, or a variant of an antibody construct); (c) measuring the viscosity of the antigen binding polypeptide variant; (d) identifying the antigen binding polypeptide variant having reduced viscosity relative to the wild type antigen binding polypeptide, thereby reducing the viscosity of the antigen binding polypeptide. In some embodiments, the method comprises (a) identifying one or more wild type amino acids in the antigen binding polypeptide that have high SAP values and high SASA ratios. In some embodiments, the method comprises the step of (b) substituting the one or more wild type amino acids identified as having high SAP values and high SASA ratios with amino acids having lower SAP values and lower SASA ratios as compared to the wild type amino acids to produce an antigen binding polypeptide variant (e.g., an antibody variant, an antigen binding fragment of an antibody variant, or a variant of an antibody construct).

In some embodiments of any of the methods described herein, the terms “parental antigen binding polypeptide” and “antigen binding polypeptide” are used interchangeably.

In some embodiments, the one or more wild type amino acids in the antigen binding polypeptide (such as the parental antigen binding polypeptide) that are identified as having high SAP values and/or high SASA ratios are surface exposed amino acids. In some embodiments, the amino acid(s) that are surface exposed are identified by structural modeling (such as computational modeling) of the antigen binding polypeptide and/or by studying a solved structure (such as solved crystal structure) of the antigen binding polypeptide. In some embodiments, the one or more wild-type amino acids in the parental antigen binding polypeptide that are identified as having high SAP values and/or high SASA ratios have large side chains. In some embodiments, a “large side chain” refers to an amino acid side chain that has a volume greater than about ~189 Å, e.g., a volume between ~189 Å and ~228 Å. In some embodiments, the one or more wild-type amino acids in the parental antigen binding polypeptide that are identified as having high SAP values and/or high SASA ratios have large, hydrophobic side chains. In some embodiments, the one or more wild-type amino acids in the parental antigen binding polypeptide that are identified as having high SAP values and/or high SASA ratios are aromatic amino acids. In some embodiments, the aromatic amino acids are selected from the group constating of: W, Y, and F. In some embodiments, the one or more wild type amino acids having large side chains or large hydrophobic side chains identified as having high SAP values and/or high SASA ratios are substituted with charged amino acids or amino acids having smaller side chains. In some embodiments, a “smaller side chain” refers to an amino acid side chain that has a volume less than about 174 Å. In some embodiments, the charged amino acid(s) or amino acid(s) having smaller side chains are selected from the group consisting of A, I, L, V, D, E, H, K, R, N, Q, S, and T. In some embodiments, the one or more aromatic amino acids identified as having high SAP values and/or high SASA ratios are substituted with charged amino acids or amino acids having smaller side chains. In some embodiments, the charged amino acid(s) or amino acid(s) having smaller side chains are selected from the group consisting of A, I, L, V, D, E, H, K, R, N, Q, S, and T. In some embodiments, the one or more wild type amino acids in the parental antigen binding polypeptide identified as having high SAP values and/or high SASA ratios have smaller side chains. In some embodiments, the one or more amino acids having smaller side chains are selected from the group consisting of: A, I, L, V, N, Q, S, G, P, C, M, or T. In some embodiments, the one or more amino acids that have smaller side chains are substituted with charged amino acids. In some embodiments, the charged amino acid(s) are selected from the group consisting of: R, K, H, D, and E. In some embodiments, the charged amino acid(s) are selected from the group consisting of: R, K, and H. In some embodiments, the one or more wild type amino acids in the parental antigen binding polypeptide identified as having high SAP values and/or high SASA ratios are substituted with an R, K, or H. In some embodiments, one of the wild type amino acids in the parental antigen binding polypeptide identified as having a high SAP value and/or a high SASA ratio is substituted with an H, e.g., to produce an antigen binding polypeptide variant comprising a single histidine substitution. In some embodiments the histidine substitution is the only amino acid substitution in the antigen binding polypeptide variant. In some embodiments, the histidine substitution is the only amino acid substitution that has been introduced into the antigen binding polypeptide variant to reduce viscosity of the antigen binding polypeptide variant relative to the parental antigen binding polypeptide.

In some embodiments, provided is a method of identifying an antigen binding polypeptide variant (e.g., an antibody variant, an antigen binding fragment of an antibody variant, or a variant of an antibody construct) having reduced viscosity relative to a parental antigen binding polypeptide (e.g., an antibody, an antigen binding fragment thereof, or an antibody construct), the method comprising: (a) identifying one or more wild type amino acids selected from the group consisting of W, Y, and F in the parental antigen binding polypeptide that have high spatial aggregation propensity (SAP) values and/or high solvent-accessible surface area (SASA) ratios; (b) substituting the one or more wild type amino acids identified as having high SAP values and/or high SASA ratios with amino acids having lower SAP values and/or lower SASA ratios as compared to the wild type amino acids; (c) measuring the viscosity of the antigen binding polypeptide variant; and (d) identifying the antigen binding polypeptide variant having reduced viscosity relative to the parental antigen binding polypeptide. In some embodiments, the method comprises (a) identifying one or more wild type amino acids in the parental antigen binding polypeptide that have high SAP values and high SASA ratios. In some embodiments, the method comprises the step of (b) substituting the one or more wild type amino acids identified as having high SAP values and high SASA ratios with amino acids having lower SAP values and lower SASA ratios as compared to the wild type amino acids. In some embodiments, the method comprises determining the SAP values and/or SASA ratios of the one or more substituted amino acids in the antigen binding polypeptide variant after step (b) and prior to step (c). In some embodiments, provided is a method of reducing viscosity of an antigen binding polypeptide (e.g., an antibody, an antigen binding fragment thereof, or an antibody construct), method comprising: (a) identifying one or more wild type amino acids in the antigen binding polypeptide selected from the group consisting of W, Y and F that have high spatial aggregation propensity (SAP) values and/or high solvent-accessible surface area (SASA) ratios; (b) substituting the one or more wild type amino acids identified as having high SAP values and/or high SASA ratios with amino acids having lower SAP values and/or lower SASA ratios as compared to the wild type amino acids to produce an antigen binding polypeptide variant (e.g., an antibody variant, an antigen binding fragment of an antibody variant, or a variant of an antibody construct); (c) measuring the viscosity of the antigen binding polypeptide variant; (d) identifying the antigen binding polypeptide variant having reduced viscosity relative to the wild type antigen binding polypeptide, thereby reducing the viscosity of the antigen binding polypeptide. In some embodiments, the one or more wild type amino acids selected from the group consisting of W, Y, and F identified as having high SAP values and/or high SASA ratios are substituted with charged amino acids or amino acids having smaller side chains. In some embodiments, the charged amino acid(s) or amino acid(s) having smaller side chains are selected from the group consisting of A, I, L, V, D, E, H, K, R, N, Q, S, and T. In some embodiments, the one or more wild type amino acids in the parental antigen binding polypeptide identified as having high SAP values and/or high SASA ratios are substituted with an R, K, or H. In some embodiments, one of the wild type amino acids in the parental antigen binding polypeptide identified as having a high SAP value and/or a high SASA ratio is substituted with an H, e.g., to produce an antigen binding polypeptide variant comprising a single histidine substitution. In some embodiments the histidine substitution is the only amino acid substitution in the antigen binding polypeptide variant. In some embodiments, the histidine substitution is the only amino acid substitution that has been introduced into the antigen binding polypeptide variant to reduce viscosity of the antigen binding polypeptide variant relative to the parental antigen binding polypeptide.

In some embodiments, provided is a method of identifying an antigen binding polypeptide variant (e.g., an antibody variant, an antigen binding fragment of an antibody variant, or a variant of an antibody construct) having reduced viscosity relative to a parental antigen binding polypeptide (e.g., an antibody, an antigen binding fragment thereof, or an antibody construct), the method comprising: (a) identifying one or more wild type amino acids selected from the group consisting of W, Y, and F in the parental antigen binding polypeptide that have high spatial aggregation propensity (SAP) values and/or high solvent-accessible surface area (SASA) ratios; (b) substituting the one or more wild type amino acids identified as having high SAP values and/or high SASA ratios with (i) Y or F wherein the wild type amino acid is W, (ii) Y or W wherein the wild type amino acid is F, and/or (iii) F or W wherein the wild type amino acid is Y; (c) measuring the viscosity of the antigen binding polypeptide variant; and (d) identifying the antigen binding polypeptide variant having reduced viscosity relative to the parental antigen binding polypeptide. In some embodiments, the method comprises (a) identifying one or more wild type amino acids in the parental antigen binding polypeptide that have high SAP values and high SASA ratios. In some embodiments, the method comprises the step of (b) substituting the one or more wild type amino acids identified as having high SAP values and high SASA ratios with amino acids having lower SAP values and lower SASA ratios as compared to the wild type amino acids. In some embodiments, the method comprises determining the SAP values and/or SASA ratios of the one or more substituted amino acids in the antigen binding polypeptide variant after step (b) and prior to step (c). In some embodiments, provided is a method of reducing viscosity of an antigen binding polypeptide (e.g., an antibody, an antigen binding fragment thereof, or an antibody construct), method comprising: (a) identifying one or more wild type amino acids in the antigen binding polypeptide selected from the group consisting of W, Y and F that have high spatial aggregation propensity (SAP) values and/or high solvent-accessible surface area (SASA) ratios; (b) substituting the one or more wild type amino acids identified as having high SAP values and/or high SASA ratios with (i) Y or F wherein the wild type amino acid is W, (ii) Y or W wherein the wild type amino acid is F, and/or (iii) F or W wherein the wild type amino acid is Y to produce an antigen binding polypeptide variant (e.g., an antibody variant, an antigen binding fragment of an antibody variant, or a variant of an antibody construct); (c) measuring the viscosity of the antigen binding polypeptide variant; (d) identifying the antigen binding polypeptide variant having reduced viscosity relative to the wild type antigen binding polypeptide, thereby reducing the viscosity of the antigen binding polypeptide.

SAP gives the effective dynamically exposed hydrophobicity of a certain patch on the surface of an antigen binding polypeptide. SAP is calculated for spherical regions centered on every atom in the antigen binding polypeptide. This gives a unique SAP value for each atom. The SAP for an amino acid residue is obtained by averaging the SAP of all its constituent atoms. Additional details regarding SAP and calculating SAP values are provided in, e.g., Chennamsetty et al. (2009) PNAS USA, 106 (29): 11937-11942; Lauer et al. (2012),J Pharm Sci, 101(1): 102-115; and elsewhere. In some embodiments, molecular dynamic simulation is used to prepare an averaged model for SAP considering the flexibility of antibody molecules in solution. See, e.g., R. Salomon-Ferrer, D. A. Case, R. C. Walker. (2013) “An overview of the Amber biomolecular simulation package.” WIREs Comput. Mol. Sci. 3, 198-210; and D. A. Case, T. E. Cheatham, III, T. Darden, H. Gohlke, R. Luo, K. M. Merz, Jr., A. Onufriev, C. Simmerling, B. Wang and R. Woods. (2005) “The Amber biomolecular simulation programs.” J. Computat. Chem. 26, 1668-1688. Exemplary molecular dynamics tools are described in, e.g., D. A. Case, H. M. Aktulga, K. Belfon, I. Y. Ben-Shalom, J. T. Berryman, S. R. Brozell, D. S. Cerutti, T. E. Cheatham, III, G. A. Cisneros, V. W. D. Cruzeiro, T. A. Darden, N. Forouzesh, G. Giamba u, T. Giese, M. K. Gilson, H. Gohlke, A. W. Goetz, J. Harris, S. Izadi, S. A. Izmailov, K. Kasavajhala, M. C. Kaymak, E. King, A. Kovalenko, T. Kurtzman, T. S. Lee, P. Li, C. Lin, J. Liu, T. Luchko, R. Luo, M. Machado, V. Man, M. Manathunga, K. M. Merz, Y. Miao, O. Mikhailovskii, G. Monard, H. Nguyen, K. A. O'Hearn, A. Onufriev, F. Pan, S. Pantano, R. Qi, A. Rahnamoun, D. R. Roe, A. Roitberg, C. Sagui, S. Schott-Verdugo, A. Shajan, J. Shen, C. L. Simmerling, N. R. Skrynnikov, J. Smith, J. Swails, R. C. Walker, J. Wang, J. Wang, H. Wei, X. Wu, Y. Wu, Y. Xiong, Y. Xue, D. M. York, S. Zhao, Q. Zhu, and P. A. Kollman (2023), Amber 2023, University of California, San Francisco; C. Tian, K. Kasavajhala, K. A. A. Belfon, L. Raguette, H. Huang, A. N. Migues J. Bickel, Y. Wang, J. Pincay, Q. Wu and C. Simmerling. (2019) “ff19SB: Amino-Acid-Specific Protein Backbone Parameters Trained against Quantum Mechanics Energy Surfaces in Solution.” J. Chem. Theory Comput. 16, 528-552; and R. Salomon-Ferrer, A. W. Goetz, D. Poole; S. Le Grand, and R. C. Walker. (2013) “Routine microsecond molecular dynamics simulations with AMBER on GPUs. 2. Explicit solvent Particle Mesh Ewald.” J. Chem. Theory Comput. 9, 3878-3888. In some embodiments, the SAP values of the one or more wild type amino acids in the parental antigen binding polypeptide identified as having high SAP values are ≥2, such as about any one of 2.0, 2.1, 2.2, 2.3, 2.4, 2.5, 3.0, 3.5, 4.0, 4.5, 5.0, 5.5, 6.0, 6.5, 7.0, 7.5, 8.0 or greater than 8.0, including any value in between. In some embodiments, the SAP values of the one or more wild type amino acids in the parental antigen binding polypeptide identified as having high SAP values based on having a SAP value of 2.3 or greater.

SASA ratio is typically calculated by methods involving the in silico rolling of a spherical probe, which approximates a water molecule, around a full-atom protein model. A variety of methods are known in the art for calculating SASA ratios of soluble proteins in their folded and unfolded states. See, e.g., Ali et al. (2014) Current Protein and Peptide Science, 15(5):456-76. In some embodiments, SASA ratios of wild type amino acids in the parental antigen binding protein are calculated according to the method described at ssbio(dot)readthedocs(dot)io/en/latest/instructions/msms(dot)html and Lee and Richards (1971) JMB 55(3): 379-400, IN3-IN4. In some embodiments, the SASA ratios of the one or more wild type amino acids in the parental antigen binding polypeptide identified as having high SASA ratios are ≥0.25, such as 0.3, 0.35, 0.4, 0.45, 0.5, 0.6, 0.7, 0.8, 0.9, 1.0 or greater than 1.0, including any value in between. In some embodiments, the high SASA ratio is 0.25 or greater. In some embodiments, the high SASA ratio is 0.5 or greater.

As discussed elsewhere herein, in some embodiments, the viscosity of the antigen binding polypeptide variant produced by substituting the one or more wild type amino acids identified as having high SAP values and/or the SASA ratios with amino acids having lower SAP values and/or lower SASA ratios as compared to the wild type amino acids is measured, e.g., to confirm whether the viscosity of the antigen binding polypeptide variant is lower than the viscosity of the parental antigen binding polypeptide. In some embodiments, the viscosity of the of the antigen binding polypeptide variant is measured (such as indirectly measured) via dynamic light scattering (DLS). As discussed in the Examples, DLS interaction parameter (k_{D}, ml/gram) can correlate with viscosity (η, cP). In some embodiments, the viscosity of the antigen binding polypeptide variant is measured via DLS at a concentration between about 1 mg/ml and about 10 mg/ml (e.g., about any one of 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or 10.0 mg/ml, including any value in between). In some embodiments, the viscosity of the antigen binding polypeptide variant is measured via cone and plate rheometry. See, e.g. Lang et al. (2020) Appl Sci, 10(1), 172 and Zhang et al. (2017) Curr Opin in Chem Eng, 16: 48-55. In some embodiments, the viscosity of the of the antigen binding polypeptide variant is measured via cone and plate rheometry, e.g., at a high concentration. In some embodiments, the high concentration is between about 50 mg/ml and about 300 mg/ml, such as about any one of 50, 60, 70, 80, 90, 100, 110, 120, 130, 140, 150, 160, 170, 180 190, 200, 210, 220, 230, 240, 250, 260, 270, 280, 290, or 300 mg/ml, including any value in between.

In some embodiments, the parental antigen binding polypeptide is an antibody, an antigen binding fragment of an antibody, or an antibody construct, wherein the antibody, fragment thereof, or antibody construct comprises one or more of a CDR-H1, a CDR-H2, a CDR-H3, a CDR-L1, a CDR-L2 and a CDR-L3, wherein one or more wild type amino acids in the one or more CDR-H1, CDR-H2, CDR-H3, CDR-L1, CDR-L2 and CDR-L3 are identified as having high SAP values and/or high SASA ratios. In some embodiments, one or more wild type amino acids in the one or more CDR-H1, CDR-H2, CDR-H3, CDR-L1, CDR-L2 and CDR-L3 are identified as having high SAP values and high SASA ratios. In some embodiments, the one or more wild type amino acids in the one or more CDR-H1, CDR-H2, CDR-H3, CDR-L1, CDR-L2 and CDR-L3 identified as having high SAP values and/or high SASA ratios are substituted with amino acids having lower SAP values and/or lower SASA ratios, e.g., as described above, to produce an antibody variant, an antigen binding fragment of an antibody variant, or an antibody construct variant. In some embodiments, the one or more wild-type amino acids in the parental antibody, antigen binding fragment thereof, or antibody construct that are identified as having high SAP values and/or high SASA ratios have large side chains. In some embodiments, the one or more wild-type amino identified as having high SAP values and/or high SASA ratios have large, hydrophobic side chains. In some embodiments, the one or more wild-type amino acids identified as having high SAP values and/or high SASA ratios are aromatic amino acids. In some embodiments, the aromatic amino acids are selected from the group constating of: W, Y, and F. In some embodiments, the one or more wild type amino acids having large side chains or large hydrophobic side chains identified as having high SAP values and/or high SASA ratios are substituted with charged amino acids or amino acids having smaller side chains. In some embodiments, the charged amino acid(s) or amino acid(s) having smaller side chains are selected from the group consisting of A, I, L, V, D, E, H, K, R, N, Q, S, and T. In some embodiments, the one or more aromatic amino acids identified as having high SAP values and/or high SASA ratios are substituted with charged amino acids or amino acids having smaller side chains. In some embodiments, the charged amino acid(s) or amino acid(s) having smaller side chains are selected from the group consisting of A, I, L, V, D, E, H, K, R, N, Q, S, and T. In some embodiments, the one or more wild type amino acids identified as having high SAP values and/or high SASA ratios have smaller side chains. In some embodiments, the one or more amino acids having smaller side chains are selected from the group consisting of: A, I, L, V, N, Q, S, G, P, C, M, or T. In some embodiments, the one or more amino acids that have smaller side chains are substituted with charged amino acids. In some embodiments, the charged amino acid(s) are selected from the group consisting of: R, K, H, D, and E. In some embodiments, the charged amino acid(s) are selected from the group consisting of: R, K, and H. In some embodiments, the one or more wild type amino acids in the parental antibody, antigen binding fragment thereof, or antibody construct identified as having high SAP values and/or high SASA ratios are substituted with an R, K, or H. In some embodiments, one of the wild type amino acids in the parental antibody, antigen binding fragment thereof, or antibody construct identified as having a high SAP value and/or a high SASA ratio is substituted with an H, e.g., to produce an antibody variant, antigen binding fragment thereof, or antibody construct variant comprising a single histidine substitution. In some embodiments the histidine substitution is the only amino acid substitution in the antibody variant, fragment thereof, or antibody construct variant. In some embodiments, the histidine substitution is the only amino acid substitution that has been introduced into the antibody variant, fragment thereof, or antibody construct variant to reduce viscosity of the antibody variant, fragment thereof, or antibody construct variant relative to the parental antibody, fragment thereof, or antibody construct.

In some embodiments, the method comprises: (a) identifying one or more wild type amino acids selected from the group consisting of W, Y, and F in one or more of the CDR-H1, the CDR-H2, the CDR-H3, the CDR-L1, the CDR-L2 and the CDR-L3 of the parental antibody, antigen binding fragment thereof, or antibody construct that have high spatial aggregation propensity (SAP) values and/or high solvent-accessible surface area (SASA) ratios; (b) substituting the one or more wild type amino acids identified as having high SAP values and/or high SASA ratios with amino acids having lower SAP values and/or lower SASA ratios as compared to the wild type amino acids to produce an antibody variant, antigen binding fragment thereof, or antibody construct variant; (c) measuring the viscosity of the variant; and (d) identifying the antibody variant, antigen binding fragment thereof, or antibody construct variant having reduced viscosity relative to the parental antibody, an antigen binding fragment of an antibody, or an antibody construct. In some embodiments, the method comprises (a) identifying one or more wild type amino acids selected from the group consisting of W, Y, and F in one or more of the CDR-H1, the CDR-H2, the CDR-H3, the CDR-L1, the CDR-L2 and the CDR-L3 of the parental antibody, antigen binding fragment thereof, or antibody construct that have high spatial aggregation propensity (SAP) values and high solvent-accessible surface area (SASA) ratios. In some embodiments, the method comprises the step of (b) substituting the one or more wild type amino acids identified as having high SAP values and high SASA ratios with amino acids having lower SAP values and lower SASA ratios as compared to the wild type amino acids to produce an antibody variant, antigen binding fragment thereof, or antibody construct variant. In some embodiments, the method comprises determining the SAP values and/or SASA ratios of the one or more substituted amino acids in the antibody variant, antigen binding fragment thereof, or antibody construct variant after step (b) and prior to step (c).

In some embodiments, provided is a method of reducing viscosity of an antibody, an antigen binding fragment thereof, or an antibody construct (or a method of identifying an antibody variant, antigen binding fragment thereof, or an antibody construct variant having reduced viscosity), the method comprising: (a) identifying one or more wild type amino acids in the antigen binding polypeptide selected from the group consisting of W, Y and F that have high spatial aggregation propensity (SAP) values and/or high solvent-accessible surface area (SASA) ratios; (b) substituting the one or more wild type amino acids identified as having high SAP values and/or high SASA ratios with amino acids having lower SAP values and/or lower SASA ratios as compared to the wild type amino acids to produce an antibody variant, an antigen binding fragment of an antibody variant, or a variant of an antibody construct; (c) measuring the viscosity of the antibody variant, or a variant of an antibody construct; (d) identifying the variant having reduced viscosity relative to the wild type antibody, antigen binding fragment thereof, or antibody construct, thereby reducing the viscosity of the antibody, antigen binding fragment thereof, or antibody construct. In some embodiments, the one or more wild type amino acids selected from the group consisting of W, Y, and F identified as having high SAP values and/or high SASA ratios are substituted with charged amino acids or amino acids having smaller side chains. In some embodiments, the charged amino acid(s) or amino acid(s) having smaller side chains are selected from the group consisting of A, I, L, V, D, E, H, K, R, N, Q, S, and T. In some embodiments, the one or more wild type amino acids identified as having high SAP values and/or high SASA ratios are substituted with an R, K, or H. In some embodiments, one of the wild type amino acids identified as having a high SAP value and/or a high SASA ratio is substituted with an H, e.g., to produce an antibody variant, antigen binding fragment thereof, or antibody construct variant comprising a single histidine substitution. In some embodiments the histidine substitution is the only amino acid substitution in the variant. In some embodiments, the histidine substitution is the only amino acid substitution that has been introduced into the antibody variant, antigen binding fragment thereof, or antibody construct variant to reduce viscosity of the variant relative to the parental antibody, antigen-binding fragment thereof, or antibody construct.

In some embodiments, provided is a method of reducing viscosity of an antibody, an antigen binding fragment thereof, or an antibody construct (or a method of identifying an antibody variant, antigen binding fragment thereof, or an antibody construct variant having reduced viscosity), the method comprising: (a) identifying one or more wild type amino acids in the antigen binding polypeptide selected from the group consisting of W, Y and F that have high spatial aggregation propensity (SAP) values and/or high solvent-accessible surface area (SASA) ratios; (b) substituting the one or more wild type amino acids identified as having high SAP values and/or high SASA ratios with (i) Y or F wherein the wild type amino acid is W, (ii) Yor W wherein the wild type amino acid is F, and/or (iii) F or W wherein the wild type amino acid is Y to produce an antibody variant, an antigen binding fragment of an antibody variant, or a variant of an antibody construct; (c) measuring the viscosity of the antibody variant, or a variant of an antibody construct; (d) identifying the variant having reduced viscosity relative to the wild type antibody, antigen binding fragment thereof, or antibody construct, thereby reducing the viscosity of the antibody, antigen binding fragment thereof, or antibody construct.

In some embodiments, the parental antigen binding polypeptide is an antibody. In some embodiments, the antibody is a therapeutic antibody. In some embodiments, the antibody is a chimeric antibody, a humanized antibody, or a human antibody. In some embodiments, the antibody is a full-length antibody. In some embodiments, the antibody is a monospecific antibody, a bispecific antibody, or a multispecific antibody. In some embodiments, the parental antigen binding polypeptide is an antibody, antigen binding fragment thereof, or antibody construct that comprises a human IgG Fc region, or a portion thereof, such as the CH2 and/or CH3 domain. In some embodiments, the human IgG constant region is an IgG1, IgG2, or IgG4 constant region.

In some embodiments, the parental antigen binding polypeptide is an antibody construct (such as an antibody-based construct), e.g., a multispecific antibody construct. Exemplary antibody constructs that find use with the methods described herein include, without limitation, CrossMabs, dual action Fabs (“DAFs”), e.g., two-in-one DAFs and four-in-one DAFs, DutaMabs, DT-IgGs, knobs-in holes (“KIH”) bispecific antibodies, e.g., common light chain KIH bispecific antibodies and KIH bispecific antibodies comprising two different light chains, SEEDbodies, TrioMabs, Dock and Lock bispecific constructs, DVD-IgGs, IgG(H)-scFvs, scFv-(H)IgGs, IgG(L)-scFvs, scFv-(L)IgGs, IgG(L, H)-Fvs, IgG(H)-Vs, V(H)-IgGs, IgG(L)-Vs, V(L)-IgGs, KIH IgG-scFabs, 2scFv-IgGs, scGv4-Igs, Zybodies, and DVI-IgGs, such as four-in-one DVI-IgGs. These and other multispecific antibody constructs are described in further detail in, e.g., Spiess et al. (2015) Mol Immunol, 67(2 Pt A):95-106; Labrijn et al. (2019) Nat Revs Drug Discovery, 18: 585-608; and elsewhere.

In some embodiments, the parental antigen binding polypeptide is an antigen binding fragment of an antibody, or a construct comprising an antigen binding fragment of an antibody. Exemplary antigen binding fragments, and constructs comprising such fragments, include, but are not limited to, e.g., Fabs, Fab₂s, such as monospecific and bispecific Fab₂s, F(ab′)₂s, such as monospecific and bispecific F(ab′)₂s, trispecific Fab₃s, scFvs, monovalent IgGs, one-armed antibodies, diabodies, triabodies, scFv-Vcs, minibodies, V_{H}Hs, V-NARs, hcIgGs, IgNARs, nanobodies, nanobody-HSAs, bispecific T-cell engagers (BiTEs), dual affinity retargeting molecules (DARTs), tandem diabodies (TandAbs), scDiabodies, scDiabody-CH3s, diabody-CH3s, triple bodies, miniantibodies, minibodies, TriBi minibodies, scFv-CH3 KIHs, Fab-scFvs, scFv-CH-VL-scFvs, F(ab′)₂, -scFv2s, scFv-KIHs, Fab-scFv-Fcs, intrabodies, and others. These and other antigen-binding fragments, and constructs comprising such fragments, are described in further detail in, e.g., Spiess et al. (2015) Mol Immunol, 67(2 Pt A):95-106; Labrijn et al. (2019) Nat Revs Drug Discovery, 18: 585-608; and elsewhere.

In some embodiments, the antigen binding polypeptide (e.g., the antibody, the antigen binding fragment of the antibody, or the antibody construct) is subject to at least one affinity maturation step (e.g., 1, 2, 3, 4, 5, 6, 7, 8, 9, 10, or more than 10 affinity maturation steps) prior to being used in a method described herein (e.g., before substitution of one or more wild type amino acids having high SAP and/or high SASA ratios with amino acids having lower SAP and/or lower SASA ratios). In some embodiments, the antigen binding polypeptide variant (e.g., the antibody variant, the antigen binding fragment of the antibody variant, or the antibody construct variant) produced by a method described herein is subject to at least one affinity maturation step (e.g., 1, 2, 3, 4, 5, 6, 7, 8, 9, 10, or more than 10 affinity maturation steps), e.g., following substitution of one or more wild type amino acids having high SAP and/or high SASA ratios with amino acids having lower SAP and/or lower SASA ratios. Details regarding Affinity maturation are provided elsewhere herein. In some embodiments, the substituted amino acids in the antigen binding polypeptide variant (i.e., the amino acids having lower SAP values and/or lower SASA ratios that were introduced into the variant to replace wild type amino acids having high SAP values and/or high SASA ratios) are fixed (such as not randomized or not further substituted) during the affinity maturation step(s). In some embodiments, one or more amino acids in the antigen binding polypeptide variant (e.g., other than the amino acids having lower SAP values and/or lower SASA ratios that replaced wild type amino acids having high SAP values and/or high SASA ratios) are randomized to produce a library of randomized variants. In some embodiments, the one or more amino acids in the randomized variants that were replaced to improve the affinity of the antigen binding polypeptide variant for its target are at or near the site of the antigen binding polypeptide variant that contacts the target (e.g., target ligand or target antigen). The library of randomized variants is then screened to identify those variants with the desired affinity for the target.

Thus, in certain embodiments, affinity maturation comprises the steps of: mutagenizing or randomizing the CDR-H1, CDR-H2, CDR-H3, CDR-L1, CDR-L2, and/or CDR-L3 of an antibody variant, an antigen binding fragment of the antibody variant, or an antibody construct variant obtained by a method herein at one or more positions to produce a library of randomized variants; contacting the library of randomized variants with a target (e.g., a target ligand or target antigen); detecting the binding of the target to one or more randomized variants; and obtaining the one or more randomized variants that specifically bind the target. In some embodiments, the randomized variant thus identified is referred to as an “affinity matured variant.” In some embodiments, the one or more amino acids in the CDR-H1, CDR-H2, CDR-H3, CDR-L1, CDR-L2, and/or CDR-L3 in the antibody variant, antigen binding fragment of the antibody variant, or antibody construct variant having lower SAP values and/or lower SASA ratios that were introduced to replace wild type amino acids having high SAP values and/or high SASA ratios are fixed and not targeted for further randomization (e.g., not further mutagenized). The methods for mutagenizing CDR-H1, CDR-H2, CDR-H3, CDR-L1, CDR-L2, and/or CDR-L3 of an antibody (or fragment antigen-binding fragment thereof) are known in the art, and they are discussed elsewhere herein. Details regarding libraries and library screens are provided elsewhere herein.

In certain embodiments, the methods described herein further comprise a step of determining the nucleic acid sequence of the antigen binding polypeptide variant obtained following at least one round of affinity maturation. In certain embodiments, the methods described herein further comprise a step producing the randomized variant (e.g., randomized antibody variant, antigen binding fragment of a randomized antibody variant, or randomized antibody construct variant). In some embodiments, the method of producing the randomized variant (e.g., randomized antibody variant, antigen binding fragment of a randomized antibody variant, or randomized antibody construct variant) is a manufacturing-scale production process (such as a fermentation process).

In some embodiments, provided herein is an antigen binding polypeptide variant (e.g., an antibody variant, an antigen binding fragment of an antibody variant, or an antibody construct variant) produced according to a method described herein. In some embodiments, provided is a composition comprising an antigen binding polypeptide variant (e.g., an antibody variant, an antigen binding fragment of an antibody variant, or an antibody construct variant) produced according to a method described herein at a concentration between about 20 mg/ml and 500 mg/ml, such as between about 30 mg/ml and about 400 mg/ml, between about 40 mg/ml and about 350 mg/ml, or between about 50 mg/ml and about 300 mg/ml.

The amino acid sequences of exemplary VH domain variants of an anti-GCGR antibody that were produced according to a method described herein are provided below.

In some embodiments, provided herein is an affinity matured antigen binding polypeptide variant (e.g., an affinity matured antibody variant, an antigen binding fragment of an affinity matured antibody variant, or an affinity matured antibody construct variant), e.g., obtained following affinity maturation of an antigen binding polypeptide variant produced according to a method described herein. In some embodiments, provided is a composition comprising an affinity matured antigen binding polypeptide variant (e.g., an affinity matured antibody variant, an antigen binding fragment of an antibody variant, or an affinity matured antibody construct variant) at a concentration between about 20 mg/ml and 500 mg/ml, such as between about 30 mg/ml and about 400 mg/ml, between about 40 mg/ml and about 350 mg/ml, or between about 50 mg/ml and about 300 mg/ml.

### Methods Using Dynamic Light Scattering (DLS) Interaction Parameter

In certain aspects, provided herein are methods comprising the use of dynamic light scattering (DLS) interaction parameter to assess viscosity of an antigen binding polypeptide. As described herein, the inventors found that there is a strong linear correlation between the DLS interaction parameter measured for an antigen binding polypeptide, such as an antigen binding polypeptide variant described herein, and viscosity. In some embodiments, the methods using DLS interaction parameter to assess viscosity are configured to enable medium-throughput screening of a plurality of antigen binding polypeptides, e.g., at least about 100 antigen binding polypeptides, such as at least about 150, 200, 250, 300, 350, 400, 450, 500, 550, 600, 650, 700, 750, 800, 850, 900, 950, or 1,000 antigen binding polypeptides.

In some embodiments, the method comprises measuring the DLS interaction parameter of a composition comprising an antigen binding polypeptide, such as an antigen binding polypeptide variant. In some embodiments, measuring the DLS interaction parameter of a composition comprising an antigen binding polypeptide comprises subjecting the composition to a DLS technique, wherein the composition comprises between about 0.1 mg/ml and about 100 mg/ml, such as between about 2 mg/ml and about 10 mg/ml, of the antigen binding polypeptide to measure each the interaction parameter using the following equation:

$$D = {D}_{0} ( 1 + {k}_{D} * c )$$

where D=translational diffusion coefficient; D₀ is the self-diffusion coefficient (the value of D at zero concentration); c=protein concentration; and k_{D}=interaction parameter. The more positive a variant's k_{D} value, the weaker the variant's self-interaction. Conversely, the more negative a variant's k_{D} value, the stronger the variant's self-interaction. See, e.g., Sorret et al. (2016) Biophysical Journal, 111: 1831-1842. Techniques for measuring DLS are known in the art and include, e.g., techniques involving fixed angle of multi-angle instruments. Moreover, there are a number of techniques for analyzing DLS data including, e.g., CUMULANT, CONTIN, and CORENN. E.g., <https://lsinstruments.ch/en/theory/dynamic-light-scattering-dls/introduction>, accessed on Sep. 1, 2023, which is hereby incorporated herein by reference in its entirety.

In some embodiments, the method comprises comparing the measured DLS interaction parameter of the antigen binding polypeptide and a standard curve and/or a reference to determine the viscosity of the antigen binding polypeptide based on the DLS interaction parameter. In some embodiments, the standard curve and/or reference comprises known viscosity and DLS interaction parameters of other polypeptides, such as variants of the antigen binding polypeptide.

In certain aspects provided herein, the methods for assessing viscosity of an antigen binding polypeptide are further paired with an assay (such as a binding assay, an activity assay, or an efficacy assay) to assess the antigen binding polypeptide, e.g., to assess the affinity of the antigen binding polypeptide with the target antigen. Such methods taught herein thus enable evaluation of viscosity and a desirable attribute of an antigen binding polypeptide (e.g., binding affinity for a target antigen, activity, or efficacy), which, e.g., can aid in therapeutic drug development to identify useful antigen binding polypeptides having desired properties, such as low viscosity and high binding affinity. Techniques for measuring binding affinity of an antigen binding polypeptide are known in the art and include techniques involving surface plasmon resonance (SPR) and bio-layer interference (BLI). E.g., Regenmortel & Azimzadeh, J Immunoassay, 21, 2000, which is hereby incorporated herein by reference in its entirety. Techniques for conducting activity and efficacy assays are known in the art and include animal model and cell-based assays. E.g., Jiang & Mire-Sluis, Biosimilars, 2018, which is hereby incorporated herein by reference in its entirety.

In certain aspects, provided herein are methods comprising: (a) performing mutagenesis of an antigen binding polypeptide based on SAP values and/or and SASA ratios as described herein (e.g., based on variants having high SAP values and/or high SASA ratios); and (b) using DLS to identify one or more mutants having a desirable viscosity (such as a low viscosity or a viscosity lower than a parent antigen binding polypeptide on which the mutagenesis was based upon) as described herein. In some embodiments, the method further comprises conducting one or more additional assays to assess a characteristic of the mutant antigen binding polypeptide, such as a binding assay, an activity assay, or an efficacy assay. For example, in some embodiments, such methods are useful to identify antigen binding polypeptide variants having a reduced viscosity and a desired (such as higher) binding affinity or activity relative to a parental antigen binding polypeptide.

### Methods of Predicting Viscosity-Affecting Amino Acids in an Antigen Binding Polypeptide

In some embodiments, provided is a method of predicting one or more amino acids in an antigen binding polypeptide that affect the viscosity of the antigen binding polypeptide. In some embodiments, the method comprising: identifying one or more amino acids in the antigen binding polypeptide that have high spatial aggregation propensity (SAP) values and/or high solvent-accessible surface area (SASA) ratios, wherein the one or more amino acids that have high spatial aggregation propensity (SAP) values and/or high solvent-accessible surface area (SASA) ratios are predicted to affect viscosity of the antigen binding polypeptide. In some embodiments, the method comprises identifying one or more amino acids in the antigen binding polypeptide that have high spatial aggregation propensity (SAP) values and high solvent-accessible surface area (SASA) ratios, wherein the one or more amino acids that have high spatial aggregation propensity (SAP) values and high solvent-accessible surface area (SASA) ratios are predicted to affect viscosity of the antigen binding polypeptide.

In some embodiments, provided herein is a method of predicting one or more amino acids in an antibody, an antigen binding fragment of an antibody, or an antibody construct that affect the viscosity of the antibody, the antigen binding fragment, or the antibody construct, wherein the antibody, the antigen binding fragment, or the antibody construct comprises one or more of a CDR-H1, a CDR-H2, a CDR-H3, a CDR-L1, a CDR-L2 and a CDR-L3, the method comprising: identifying one or more amino acid positions in the one or more of the CDR-H1, the CDR-H2, the CDR-H3, the CDR-L1, the CDR-L2 and the CDR-L3 that have high spatial aggregation propensity (SAP) values and/or high solvent-accessible surface area (SASA) ratios, wherein the one or more amino acid positions that have the high spatial aggregation propensity (SAP) values and/or the high solvent-accessible surface area (SASA) ratios are predicted to affect viscosity of the antibody, the antigen binding fragment, or the antibody construct. In some embodiments, the method comprises: identifying one or more amino acid positions in the one or more of the CDR-H1, the CDR-H2, the CDR-H3, the CDR-L1, the CDR-L2 and the CDR-L3 that have high spatial aggregation propensity (SAP) values and high solvent-accessible surface area (SASA) ratios, wherein the one or more amino acid positions that have the high spatial aggregation propensity (SAP) values and the high solvent-accessible surface area (SASA) ratios are predicted to affect viscosity of the antibody, the antigen binding fragment, or the antibody construct.

In some embodiments, the antibody is a therapeutic antibody. In some embodiments, the antibody is a chimeric antibody, a humanized antibody, or a human antibody. In some embodiments the antibody is a monospecific antibody, a bispecific antibody, or a multispecific antibody. In some embodiments, the antibody is a full-length antibody. In some embodiments, the antibody comprises a human IgG Fc region, or a portion thereof, such as the CH2 and/or CH3 domain. In some embodiments, the human IgG constant region is an IgG1, IgG2, or IgG4 constant region. In some embodiments, the antibody fragment is a Fab, a F(ab′)₂, a trispecific Fab₃, an scFv, a monovalent IgG, a diabody, a triabody, an scFv-Vc, a minibody, a V_{H}H, a V-NAR, an hcIgG, or an IgNAR. Other exemplary antibody fragments that find used with the methods include, but are not limited to, those described in detail elsewhere herein. In some embodiments, the antibody construct is, e.g., CrossMab, a dual action Fab (DAF), a DVD-IgG, or a knob-in-hole bispecific antibody. Other exemplary antibody constructs that find used with the methods include, but are not limited to, those described in detail elsewhere herein.

### Libraries and Library Screens

In some embodiments, provided herein is a library comprising a plurality of antigen binding polypeptide variants, wherein at least one variant in the library comprises one or more amino acid substitutions relative to a parental antigen binding polypeptide, wherein the one or more amino acid substitutions relative to the parental antigen binding polypeptide replace one or more wild type amino acids identified as having high SAP values and/or high SASA ratios with amino acids having lower SAP values and/or lower SASA ratios, as compared to the wild type amino acids. In some embodiments, each variant in the library comprises one or more amino acid substitutions relative to a parental antigen binding polypeptide, wherein the one or more amino acid substitutions relative to the parental antigen binding polypeptide replace one or more wild type amino acids identified as having high SAP values and/or high SASA ratios with amino acids having lower SAP values and/or lower SASA ratios, as compared to the wild type amino acids. In some embodiments, the library is screened to identify antigen binding polypeptide variants with reduced viscosity, as compared to a parental antigen binding polypeptide. In some embodiments, the library is screened to identify antigen binding polypeptide variants having reduced viscosity, as compared to a parental antigen binding polypeptide, and affinities for a target (e.g., target ligand or target antigen) that are comparable to, at least as high as, or improved as compared to the affinity of the parental antigen binding polypeptide for the target. In some embodiments, an affinity for a target that is “comparable” to the affinity of the parental antigen binding polypeptide for the target is an affinity that is within 5-fold (such as any one of about 4.5-, 4.0-, 3.5-, 3.0-, 2.5-, 2.0-, 1.5- or less than 1.5-fold, including any value in between) of the affinity of the parental antigen binding polypeptide for the target.

In some embodiments, the library is a polypeptide library (such as a plurality of antigen binding polypeptide variants, e.g., antibody variants, antigen binding fragments of antibody variants, or antibody construct variants). In some embodiments, the polypeptide library is a polypeptide display library. Such polypeptide display libraries can be screened to select and/or evolve antigen binding polypeptide variants with desired properties for a wide variety of utilities, including but not limited to therapeutic, prophylactic, veterinary, diagnostic, reagent, or material applications. In certain embodiments, the library is a nucleic acid library, wherein each nucleic acid (or a group of nucleic acids) encodes a different antigen binding polypeptide variant. In some embodiments, the library is a plurality of host cells (e.g., prokaryotic or eukaryotic host cells) each comprising (and, e.g., expressing) a different nucleic acid (or a group of nucleic acids), wherein each different nucleic acid (or a group of nucleic acids) encodes a different antigen binding polypeptide variant.

In certain embodiments, a plurality of antigen binding polypeptide variants in a library described herein comprises at least 2, 3, 4, 5, 10, 30, 100, 250, 500, 750, 1000, 2500, 5000, 7500, 10000, 25000, 50000, 75000, 100000, 250000, 500000, 750000, 1000000, 2500000, 5000000, 7500000, 10000000, or more than 10000000 different antigen binding polypeptide variants (such as unique antigen binding polypeptide variants), including any value in between. In certain embodiments, the library of antigen binding polypeptide variants has a sequence diversity of about 2, about 5, about 10, about 50, about 100, about 250, about 500, about 750, about 10³, about 10⁴, about 10⁵, about 10⁶, about 10⁷, about 10⁸, about 10⁹, about 10¹⁰, about 10¹¹, about 10¹², about 10¹³, about 10¹⁴, or more than about 10¹⁴ (such as about 10¹⁵ or about 10¹⁶), including any value in between.

In certain embodiments, the library generated via genetic engineering. A variety of methods for mutagenesis and subsequent library construction have been previously described (along with appropriate methods for screening or selection). Such mutagenesis methods include, but are not limited to, e.g., error-prone PCR, loop shuffling, or oligonucleotide-directed mutagenesis, random nucleotide insertion or other methods prior to recombination. Further details regarding these methods are described in, e.g., Abou-Nadler et al. (2010) Bioengineered Bugs 1, 337-340; Firth et al. (2005) Bioinformatics 21, 3314-3315; Cirino et al. (2003) Methods Mol Biol 231, 3-9; Pirakitikulr (2010) Protein Sci 19, 2336-2346; Steffens et al. (2007) J. Biomol Tech 18, 147-149; and others. Accordingly, in certain embodiments, provided are multispecific antigen-binding protein libraries generated via genetic engineering techniques.

In certain embodiments, the library is generated via in vitro translation. Briefly, in vitro translation entails cloning the polypeptide-coding sequence(s) into a vector containing a promoter, producing mRNA by transcribing the cloned sequence(s) with an RNA polymerase, and synthesizing the polypeptide by translation of this mRNA in vitro, e.g., using a cell-free extract. A desired antigen binding polypeptide variant can be generated simply by altering the cloned polypeptide-coding sequence. Many mRNAs can be translated efficiently in wheat germ extracts or in rabbit reticulocyte lysates. Further details regarding in vitro translation are described in, e.g., Hope et al. (1985) Cell 43, 177-188; Hope et al. (1986) Cell 46, 885-894; Hope et al. (1987) EMBO J 6, 2781-2784; Hope et al. (1988) Nature 333, 635-640; and Melton et al. (1984) Nucl. Acids Res. 12, 7057-7070.

Accordingly, provided in some embodiments is a plurality of nucleic acid molecules encoding a polypeptide display library described herein. An expression vector operably linked to the plurality of nucleic acid molecules is also provided herein. Also provided is a method of making a library provided herein by providing a plurality of nucleic acids encoding a plurality of antigen binding domains described herein, and expressing the nucleic acids.

In certain embodiments, a library provided herein is generated via chemical synthesis. Methods of solid phase and liquid phase peptide synthesis are well known in the art and described in detail in, e.g., Fmoc Solid Phase Peptide Synthesis, A Practical Approach, (W. C. Chan, P. D. White Eds), Oxford University Press, 2000; Solid Phase Synthesis, A Practical Guide, (S. F. Kates, F Albericio Eds), Marcel Dekker, 2000; P. Seneci, Solid-Phase Synthesis and Combinatorial Technologies, John Wiley & Sons, 2000; Synthesis of Peptides and Peptidomimetics (M. Goodman, Editor-in-chief, A. Felix, L. Moroder, C. Tmiolo Eds), Thieme, 2002; N. L. Benoiton, Chemistry of Peptide Synthesis, CRC Press, 2005; Methods in Molecular Biology, 298, Peptide Synthesis and Applications, (J. Howl Ed) Humana Press, 2005; and Amino Acids, Peptides and Proteins in Organic Chemistry, Volume 3, Building Blocks, Catalysts and Coupling Chemistry, (A. B. Hughs, Ed.) Wiley-VCH, 2011. Accordingly, in certain embodiments, provided is a multispecific antigen-binding protein library generated via chemical synthesis techniques.

In certain embodiments, a library provided herein is a display library. In certain embodiments, the display library is a phage display library, a phagemid display library, a virus display library, a bacterial display library, a yeast display library, a λgt11 library, a CIS display library, and in vitro compartmentalization library, or a ribosome display library. Methods of making and screening such display libraries are well known to those of skill in the art and described in, e.g., Molek et al. (2011) Molecules 16, 857-887; Boder et al., (1997) Nat Biotechnol 15, 553-557; Scott et al. (1990) Science 249, 386-390; Brisette et al. (2007) Methods Mol Biol 383, 203-213; Kenrick et al. (2010) Protein Eng Des Sel 23, 9-17; Freudl et al. (1986) J Mol Biol 188,491-494; Getz et al. (2012) Methods Enzymol 503, 75-97; Smith et al. (2014) Curr Drug Discov Technol 11, 48-55; Hanes, et al. (1997) Proc Natl Acad Sci USA 94,4937-4942; Lipovsek et al., (2004) J Imm Methods 290, 51-67; Ullman et al. (2011) Brief. Funct. Genomics, 10, 125-134; Odegrip et al. (2004) Proc Natl Acad Sci USA 101, 2806-2810; and Miller et al. (2006) Nat Methods 3, 561-570.

In certain embodiments, a library provided herein is an RNA-protein fusion library generated, for example, by the techniques described in Szostak et al., U.S. Pat. Nos. 6,258,558, 6,261,804, 5,643,768, and 5,658,754. In certain embodiments, a library provided herein is a DNA-protein library, as described, for example, in U.S. Pat. No. 6,416,950.

### Methods of Screening

In some embodiments, a library described herein is screened to identify an antigen binding polypeptide variant (e.g., antibody variant, antigen binding fragment of an antibody variant, or an antibody construct variant) having reduced viscosity, as compared to a parental antigen binding polypeptide (e.g., parental antibody, antigen binding fragment of a parental antibody, or a parental antibody construct). In some embodiments, a library provided herein can be screened to identify an antigen binding polypeptide variant (e.g., antibody variant, antigen binding fragment of an antibody variant, or an antibody construct variant) having low viscosity (e.g., reduced viscosity as compared to the parental antigen binding protein) and/or having an affinity for a target antigen that is comparable to, at least as high as, or improved as compared to the affinity of the parental antigen binding polypeptide (e.g., parental antibody, antigen binding fragment of a parental antibody, or a parental antibody construct) for the target antigen.

In certain embodiments, screening the library comprises the steps of: contacting a library described herein under a condition that allows binding of a target of interest with an antigen binding polypeptide variant in the library that specifically binds the target; detecting the binding of the target with the antigen binding polypeptide variant that specifically binds the target (e.g., detecting a complex comprising the target and the antigen binding polypeptide variant that specifically binds the target); and obtaining the antigen binding polypeptide variant that specifically binds the target. In some embodiments, the method further comprises measuring the viscosity of the antigen binding polypeptide variant thus obtained, e.g., according to a method described herein, to identify an antigen binding polypeptide variant having low viscosity (e.g., a viscosity of less than about 50 cP, less than about 20 cP, or less than about 10 cP, when measured at high concentration, such as at 180 mg/mL, 20 mM histidine acetate, pH 5.5, 25° C.) or reduced viscosity compared to the parental antigen binding polypeptide.

In some embodiments, screening the library further comprises subjecting the antigen binding polypeptide variant identified via library screen or an antigen binding polypeptide variant obtained by using a method herein to at least one affinity maturation step (e.g., 1, 2, 3, 4, 5, 6, 7, 8, 9, 10, or more than 10 affinity maturation steps). In some embodiments, an antigen binding polypeptide is subject to at least one affinity maturation step (e.g., 1, 2, 3, 4, 5, 6, 7, 8, 9, 10, or more than 10 affinity maturation steps) to obtain an affinity matured antigen binding polypeptide variant, and the affinity matured antigen binding polypeptide variant is used in a method described herein or in a library or library screen described herein. Affinity maturation is a process by which an antigen binding polypeptide variant (e.g., an antibody variant, an antigen binding fragment of the antibody variant, or an antibody construct variant) produced by a method provided herein or by screening a library provided herein is subject to a scheme that selects for increased affinity for a target (e.g., target ligand or target antigen) (see Wu et al. (1998) Proc Natl Acad Sci USA. 95, 6037-42). Details regarding affinity maturation of antibodies are also detailed in, e.g., Merchant et al. (2013) Proc Natl Acad Sci USA. 110(32): E2987-96; Julian et al. (2017) Scientific Reports. 7: 45259; Tiller et al. (2017) Front. Immunol. 8: 986; Koenig et al. (2017) Proc Natl Acad Sci USA. 114(4): E486-E495; Yamashita et al. (2019) Structure. 27, 519-527; Payandeh et al. (2019) J Cell Biochem. 120: 940-950; Richter et al. (2019) mAbs. 11(1): 166-177; and Cisneros et al. (2019) Mol. Syst. Des. Eng. 4: 737-746.

In some embodiments, the substituted amino acids in the antigen binding polypeptide variant (i.e., the amino acids having lower SAP values and/or lower SASA ratios that were introduced into the variant to replace wild type amino acids having high SAP values and/or high SASA ratios) are fixed (such as not randomized or not further substituted) during the affinity maturation step(s). In some embodiments, one or more amino acids in the antigen binding polypeptide variant (e.g., other than the amino acids having lower SAP values and/or lower SASA ratios that replaced wild type amino acids having high SAP values and/or high SASA ratios) are randomized to produce a library of randomized variants. In some embodiments, the one or more amino acids in the randomized variants are replaced to improve the affinity of the antigen binding polypeptide variant for its target. In some embodiments, the one or more amino acids in the randomized variants that are replaced to improve the affinity of the antigen binding polypeptide variant for its target are at or near the site of the antigen binding polypeptide variant that contacts the target (e.g., target ligand or target antigen). The library of randomized variants is then screened to identify those variants with the desired affinity for the target. In some embodiments, a variant obtained following an affinity maturation is referred to as an “affinity matured variant.”

In some embodiments, wherein the antigen binding polypeptide is an antibody, antigen binding fragment thereof, or an antibody construct, or a variant of any of the preceding obtained by using a method herein, the one or more amino acids that are randomized during affinity maturation are in the V_{H} and/or V_{L}. In some embodiments, the one or more amino acids that are randomized during affinity maturation are in the CDR-H1, CDR-H2, CDR-H3, CDR-L1, CDR-L2, and/or CDR-L3. Thus, in certain embodiments, the methods described herein comprise (such as further comprise) the steps of mutagenizing or randomizing the V_{H} and/or V_{L} (e.g., the CDR-H1, CDR-H2, CDR-H3, CDR-L1, CDR-L2, and/or CDR-L3) of an antibody variant, an antigen binding fragment of the antibody variant, or an antibody construct variant obtained by a method herein at one or more positions to produce a library of randomized variants; contacting the library of randomized variants with a target (e.g., a target ligand or target antigen); detecting the binding of the target to one or more randomized variants; and obtaining the one or more randomized variants that specifically bind the target. In some embodiments, the one or more amino acids in the CDR-H1, CDR-H2, CDR-H3, CDR-L1, CDR-L2, and/or CDR-L3 of the antibody variant, antigen binding fragment of the antibody variant, or antibody having lower SAP values and/or lower SASA ratios that were introduced into the variant to replace wild type amino acids having high SAP values and/or high SASA ratios are fixed (e.g., are not targeted for further randomization or mutagenesis). In some embodiments, the one or more amino acids in the CDR-H1, CDR-H2, CDR-H3, CDR-L1, CDR-L2, and/or CDR-L3 in the antibody variant, antigen binding fragment of the antibody variant, or antibody having lower SAP values and/or lower SASA ratios that were introduced into the variant to replace wild type amino acids having high SAP values and/or high SASA ratios are not fixed (e.g., are targeted for further randomization or mutagenesis). In certain embodiments, affinity matured antibody variants, antigen binding fragments thereof, or affinity matured antibody construct variants comprise at least one or at least two randomized CDRs which were not previously randomized. In some embodiments, an affinity matured antibody variant, antigen binding fragment thereof, or affinity matured antibody construct variant obtained following two or more rounds of affinity maturation have low viscosity (e.g., lower viscosity as compared to the parental antibody, fragment, or construct) and/or bind the target of interest with affinity that is at least as high as that of an affinity matured antibody variant, antigen binding fragment thereof, or affinity matured antibody construct variant obtained following one round of affinity maturation. In some embodiments, an affinity matured antibody variant, antigen binding fragment thereof, or affinity matured antibody construct variant that has undergone two or more rounds of affinity maturation has lower viscosity than the parental antibody, antigen binding fragment thereof, or parental antibody construct and binds the target of interest with an affinity that is at comparable to, least as high as, or improved over the affinity of the parental antibody, antigen binding fragment thereof, or parental antibody construct for binding the target of interest.

In some embodiments, the method further comprises measuring the viscosity of the affinity matured antigen binding polypeptide variant (i.e., the variant obtained following the least one affinity maturation step), e.g., according to a method described herein, to identify an affinity matured antigen binding polypeptide variant having high affinity for a target of interest (such as an affinity for a target of interest that is comparable to, at least as high as, or improved as compared to the affinity of a parental antigen binding polypeptide for the target) and low viscosity (such as reduced viscosity compared to a parental antigen binding polypeptide). In some embodiments, low viscosity refers to a viscosity of less than about 50 cP, less than about 20 cP, or less than about 10 cP, when measured at high concentration, such as at 180 mg/mL, 20 mM histidine acetate, pH 5.5, 25° C.

In some embodiments, the affinity matured variant is subject to further characterization. For example, in some embodiments, the further characterization comprises (a) identifying one or more amino acids in the affinity matured variant that have high spatial aggregation propensity (SAP) values and/or high solvent-accessible surface area (SASA) ratios; (b) substituting the amino acids identified as having high SAP values and/or high SASA ratios with amino acids having lower SAP values and/or lower SASA ratios as compared to the parental affinity matured variant to produce a substituted affinity matured variant (c) measuring the viscosity of the substituted affinity matured variant; (d) identifying the substituted affinity matured variant having reduced viscosity relative to the parental affinity matured variant. In some embodiments, the affinity of the substituted affinity matured variant for its target is measured, e.g., according to a method described elsewhere herein. IN some embodiments, the substituted affinity matured variant has and low viscosity (such as reduced viscosity compared to a parental antigen binding polypeptide) and high affinity for a target of interest (such as an affinity for a target of interest that is comparable to, at least as high as, or improved as compared to the affinity of a parental affinity matured variant for the target). In some embodiments, the substituted affinity matured variant is subject to one or more further affinity maturation steps (e.g., 1, 2, 3, 4, 5, 6, 7, 8, 9, 10, or more than 10 further affinity maturation steps).

The methods for mutagenizing an antigen binding polypeptide (e.g., one or more of the V_{H} and/or V_{L} or one or more of the CDR-H1, CDR-H2, CDR-H3, CDR-L1, CDR-L2, and/or CDR-L3 of an antibody variant, antigen binding fragment thereof, or antibody construct variant obtained by screening a library herein or by using a method described herein) are known in the art, and may include, for example, random mutagenesis, CDR walking mutagenesis or sequential and parallel optimization, mutagenesis by structure-based rational design, site-specific mutagenesis, enzyme-based mutagenesis, chemical-based mutagenesis, and gene synthesis methods for synthetic antibody gene production. See, e.g., Yang et al., 1995, CDR Walking Mutagenesis for the Affinity Mutation of a Potent Human Anti-HIV-1 Antibody into the Picomolar Range, J. Mol. Biol. 254:392-40, and Lim et al., 2019, Review: Cognizance of Molecular Methods for the Generation of Mutagenic Phage Display Antibody Libraries for Affinity Maturation, Int. J. Mol. Sci, 20:1861, the contents of which are both incorporated by reference herein in their entireties.

A library of antigen binding polypeptide variants described herein may be screened by any technique known in the art for evolving new or improved binding proteins that specifically bind a target ligand. In certain embodiments, the target ligand is immobilized on a solid support (such as a column resin or microtiter plate well), and the target ligand is contacted with a library of candidate multispecific antigen-binding proteins (such as any library described herein). Selection techniques can be, for example, phage display (Smith (1985) Science 228, 1315-1317), mRNA display (Wilson et al. (2001) Proc Natl Acad Sci USA 98: 3750-3755) bacterial display (Georgiou, et al. (1997) Nat Biotechnol 15:29-34.), yeast display (Boder and Wittrup (1997) Nat. Biotechnol. 15:553-5577) or ribosome display (Hanes and Plückthun (1997) Proc Natl Acad Sci USA 94:4937-4942 and WO2008/068637).

In certain embodiments, the library of antigen binding domain polypeptide variants is a phage display library. In certain embodiments, provided is a phage particle displaying an antigen binding domain variant described herein. In certain embodiments, provided is a phage particle displaying an antigen binding domain variant described herein that is capable of binding to a target ligand.

Phage display is a technique by which a plurality of multispecific antigen-binding protein variants are displayed as fusion proteins to the coat protein on the surface of bacteriophage particles (Smith, G. P. (1985) Science, 228:1315-7; Scott, J. K. and Smith, G. P. (1990) Science 249: 386; Sergeeva, A., et al. (2006) Adv. Drug Deliv. Rev. 58:1622-54). The utility of phage display lies in the fact that large libraries of selectively randomized protein variants (or randomly cloned cDNAs) can be rapidly and efficiently sorted for those sequences that bind to a target molecule with high affinity.

Display of peptides (Cwirla, S. E. et al. (1990) Proc. Natl. Acad. Sci. USA, 87:6378) or protein (Lowman, H. B. et al. (1991) Biochemistry, 30:10832; Clackson, T. et al. (1991) Nature, 352: 624; Marks, J. D. et al. (1991), J Mol. Biol., 222:581; Kang, A. S. et al. (1991) Proc. Natl. Acad. Sci. USA, 88:8363) libraries on phage have been used for screening millions of polypeptides or oligopeptides for ones with specific binding properties (Smith, G. P. (1991) Current Opin. Biotechnol., 2:668; Wu et al. (1998) Proc Natl Acad Sci USA. May 95, 6037-42). Polyvalent phage display methods have been used for displaying small random peptides and small proteins through fusions to either gene III or gene VIII of filamentous phage. (Wells and Lowman, Curr. Opin. Struct. Biol., 3:355-362 (1992), and references cited therein.) In a monovalent phage display, a protein or peptide library is fused to a gene III or a portion thereof, and expressed at low levels in the presence of wild type gene III protein so that phage particles display one copy or none of the fusion proteins. Avidity effects are reduced relative to polyvalent phage so that sorting is on the basis of intrinsic ligand affinity, and phagemid vectors are used, which simplify DNA manipulations. (Lowman and Wells, Methods: A companion to Methods in Enzymology, 3:205-0216 (1991).)

Sorting phage libraries of antigen binding polypeptide variants entails the construction and propagation of a large number of variants, a procedure for affinity purification using the target ligand, and a means of evaluating the results of binding enrichments (see for example, U.S. Pat. Nos. 5,223,409, 5,403,484, 5,571,689, and 5,663,143).

Most phage display methods use filamentous phage (such as M13 phage). Lambdoid phage display systems (see WO1995/34683, U.S. Pat. No. 5,627,024), T4 phage display systems (Ren et al. (1998) Gene 215:439; Zhu et al. (1998) Cancer Research, 58:3209-3214; Jiang et al., (1997) Infection & Immunity, 65:4770-4777; Ren et al. (1997) Gene, 195:303-311; Ren (1996) Protein Sci., 5:1833; Efimov et al. (1995) Virus Genes, 10:173) and T7 phage display systems (Smith and Scott (1993) Methods in Enzymology, 217: 228-257; U.S. Pat. No. 5,766,905) are also known.

Many other improvements and variations of the basic phage display concept have now been developed. These improvements enhance the ability of display systems to screen peptide libraries for binding to selected target molecules and to display functional proteins with the potential of screening these proteins for desired properties. Combinatorial reaction devices for phage display reactions have been developed (WO 1998/14277) and phage display libraries have been used to analyze and control bimolecular interactions (WO 1998/20169; WO 1998/20159) and properties of constrained helical peptides (WO 1998/20036). WO 1997/35196 describes a method of isolating an affinity ligand in which a phage display library is contacted with one solution in which the ligand will bind to a target molecule and a second solution in which the affinity ligand will not bind to the target molecule, to selectively isolate binding ligands. WO 1997/46251 describes a method of biopanning a random phage display library with an affinity purified antibody and then isolating binding phage, followed by a micropanning process using microplate wells to isolate high affinity binding phage. Such method can be applied to the libraries of antigen binding polypeptide variants disclosed herein. The use of Staphylococcus aureus protein A as an affinity tag has also been reported (Li et al. (1998) Mol Biotech. 9:187). WO 1997/47314 describes the use of substrate subtraction libraries to distinguish enzyme specificities using a combinatorial library which may be a phage display library. Additional methods of selecting specific binding proteins are described in U.S. Pat. Nos. 5,498,538, 5,432,018, and WO 1998/15833. Methods of generating peptide libraries and screening these libraries are also disclosed in U.S. Pat. Nos. 5,723,286, 5,432,018, 5,580,717, 5,427,908, 5,498,530, 5,770,434, 5,734,018, 5,698,426, 5,763,192, and 5,723,323.

An antibody variant, an antigen binding fragment thereof, or an antibody construct variant produced using by screening a library described herein or by using a method described herein, or an affinity matured variant of any of the preceding can be characterized for its physical/chemical properties and biological functions by various assays known in the art. Such assays include, but are not limited to, N-terminal sequencing, amino acid analysis, non-denaturing size exclusion high pressure liquid chromatography (HPLC), mass spectrometry, ion exchange chromatography and papain digestion. In some embodiments, the viscosity of the antibody variant, antigen binding fragment thereof, antibody construct variant, or affinity matured variant of any of the preceding is measured, e.g., using a method known in the art and/or described elsewhere herein.

In certain embodiments, the antibody variant, antigen binding fragment thereof, or antibody construct variant obtained by screening a library described herein or by using a method described herein, or an affinity matured variant of any of the preceding is analyzed for its biological activity. In some embodiments, the antibody variant, antigen binding fragment thereof, or antibody construct variant is tested for its antigen-binding activity. Antigen-binding assays that are known in the art and can be used herein include, without limitation, any direct or competitive binding assays using techniques such as western blots, radioimmunoassays, ELISA (enzyme-linked immunosorbent assay), “sandwich” immunoassays, immunoprecipitation assays, fluorescent immunoassays, and protein A immunoassays.

### Production and Purification of Antigen Binding Polypeptide Variants

In some embodiments, the method further comprises producing the affinity matured antigen binding polypeptide variant (e.g., antibody variant, antigen binding fragment of an antibody variant, or an antibody construct variant). In some embodiments, the method of producing the affinity-matured variant (e.g., affinity matured antibody variant, antigen binding fragment of an affinity-matured antibody variant, or affinity-matured antibody construct variant) is a manufacturing-scale production process (such as a fermentation process). An antigen binding polypeptide variant obtained by screening a library herein, by using a method herein, or by affinity maturation of a variant obtained screening a library or using a method described herein may be produced by any means known in the art. Exemplary techniques for antigen binding polypeptide variant production are described below; however these exemplary techniques are provided for illustrative purposes only and are not intended to be limiting.

An antigen binding polypeptide variant obtained by screening a library herein, by using a method herein, or by affinity maturation of a variant obtained screening a library or using a method described herein can be produced using recombinant methods. For recombinant production of an antigen binding polypeptide variant, nucleic acid(s) encoding the antigen binding polypeptide variant are isolated and inserted into replicable vector(s) for further cloning (amplification of the DNA) or for expression. DNA encoding the antigen binding polypeptide variant may be readily isolated and sequenced using conventional procedures (e.g., by using oligonucleotide probes that are capable of binding specifically to genes encoding the heavy and light chains of the antibody). Many vectors are available. The vector components generally include, but are not limited to, one or more of the following: a signal sequence, an origin of replication, one or more selectable marker genes, an enhancer element, a promoter, and a transcription termination sequence.

An antigen binding polypeptide variant obtained by screening a library herein, by using a method herein, or by affinity maturation of a variant obtained screening a library or using a method described herein can be produced recombinantly as a fusion polypeptide with a heterologous polypeptide, e.g., a signal sequence or other polypeptide having a specific cleavage site at the N-terminus of the mature protein or polypeptide. The heterologous signal sequence selected can be one that is recognized and processed (e.g., cleaved by a signal peptidase) by the host cell. For prokaryotic host cells that do not recognize and process a native antibody signal sequence, the signal sequence is substituted by a prokaryotic signal sequence selected, for example, from alkaline phosphatase, penicillinase, lpp, or heat-stable enterotoxin II leaders. For yeast secretion the native signal sequence may be substituted by, e.g., the yeast invertase leader, a factor leader (including Saccharomyces and Kluyveromyces α-factor leaders), or acid phosphatase leader, the C. albicans glucoamylase leader, etc. In mammalian cell expression, mammalian signal sequences as well as viral secretory leaders, for example, the herpes simplex gD signal, are available.

Both expression and cloning vectors contain a nucleic acid sequence that enables the vector to replicate in one or more selected host cells, e.g., to allow the vector to replicate independently of the host chromosomal DNA. This sequence can include origins of replication or autonomously replicating sequences. Such sequences are well known for a variety of bacteria, yeast, and viruses. Generally, the origin of replication component is not needed for mammalian expression vectors (the SV40 origin may be used because it contains the early promoter).

Expression and cloning vectors can contain a selection gene or selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media. Examples of dominant selection use the drugs neomycin, mycophenolic acid and hygromycin. Another example of suitable selectable markers for mammalian cells are those that enable the identification of cells competent to take up antigen binding polypeptide variant-encoding nucleic acid(s), such as DHFR, glutamine synthetase (GS), thymidine kinase, metallothionein-I and -II, preferably primate metallothionein genes, adenosine deaminase, ornithine decarboxylase, and the like. For example, a Chinese hamster ovary (CHO) cell line deficient in endogenous DHFR activity transformed with the DHFR gene is identified by culturing the transformants in a culture medium containing methotrexate (Mtx), a competitive antagonist of DHFR.

Alternatively, host cells (particularly wild-type hosts that contain endogenous DHFR) transformed or co-transformed with DNA sequences encoding an antibody of interest, wild-type DHFR gene, and another selectable marker such as aminoglycoside 3′-phosphotransferase (APH) can be selected by cell growth in medium containing a selection agent for the selectable marker such as an aminoglycosidic antibiotic, e.g., kanamycin, neomycin, or G418.

Expression and cloning vectors generally contain a promoter that is recognized by the host organism and is operably linked to nucleic acid(s) encoding an antigen binding polypeptide variant. Promoters suitable for use with prokaryotic hosts include the phoA promoter, β-lactamase and lactose promoter systems, alkaline phosphatase promoter, a tryptophan (trp) promoter system, and hybrid promoters such as the tac promoter. However, other known bacterial promoters are suitable. Promoter sequences are known for eukaryotes. Yeast promoters are well known in the art and can include inducible promoters/enhancers regulated by growth conditions. Virtually all eukaryotic genes have an AT-rich region located approximately 25 to 30 bases upstream from the site where transcription is initiated. Examples include without limitation the promoters for 3-phosphoglycerate kinase or other glycolytic enzymes, such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. Antigen binding polypeptide variant transcription from vectors in mammalian host cells can be controlled, for example, by promoters obtained from the genomes of viruses. The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment. Alternatively, the Rous Sarcoma Virus long terminal repeat can be used as the promoter.

Transcription of a DNA encoding an antigen binding polypeptide variant obtained by screening a library herein, by using a method herein, or by subjecting a variant obtained by screening a library or using a method herein to affinity maturation by higher eukaryotes is often increased by inserting an enhancer sequence into the vector. Many enhancer sequences are now known from mammalian genes (globin, elastase, albumin, α-fetoprotein, and insulin). Typically, however, one will use an enhancer from a eukaryotic cell virus.

Expression vectors used in eukaryotic host cells (yeast, fungi, insect, plant, animal, human, or nucleated cells from other multicellular organisms) will also contain sequences necessary for the termination of transcription and for stabilizing the mRNA.

Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, etc. In addition to prokaryotes, eukaryotic microbes such as filamentous fungi or yeast are suitable cloning or expression hosts for antigen binding polypeptide variant-encoding vectors. Saccharomyces cerevisiae, or common baker's yeast, is the most commonly used among lower eukaryotic host microorganisms. Certain fungi and yeast strains may be selected in which glycosylation pathways have been “humanized,” resulting in the production of an antigen binding polypeptide variant with a partially or fully human glycosylation pattern. See, e.g., Li et al., Nat. Biotech. 24:210-215 (2006).

Plant cell cultures of cotton, corn, potato, soybean, petunia, tomato, duckweed (Leninaceae), alfalfa (M. truncatula), and tobacco can also be utilized as hosts.

Suitable host cells for the expression of glycosylated antigen binding polypeptide variant are also derived from multicellular organisms (invertebrates and vertebrates). Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified.

Vertebrate cells may be used as hosts, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J Gen Virol. 36:59 (1977)); baby hamster kidney cells (BHK, ATCC CCL 10); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N. Y. Acad. Sci. 383:44-68 (1982)); MRC 5 cells; FS4 cells; and a human hepatoma line (Hep G2). Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J., 2003), pp. 255-268. In some embodiments, the host cell is a CHO-K1 cell. The CHO-K1 cell line is a subclone of CHO cell line (see, e.g., www(dot)phe-culturecollections(dot)org(dot)uk/media/128263/chok1-cell-line-profile(dot)pdf and web(dot)expasy(dot)org/cellosaurus/CVCL_0214.

The host cells may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to one of skill in the art.

When using recombinant techniques, the antigen binding polypeptide variant can be produced intracellularly, in the periplasmic space, or directly secreted into the medium. If the antigen binding polypeptide variant is produced intracellularly, as a first step, the particulate debris, either host cells or lysed fragments, are removed, for example, by centrifugation or ultrafiltration. Carter et al., Bio Technology 10:163-167 (1992) describe a procedure for isolating antibodies which are secreted to the periplasmic space of E. coli.

The antigen binding polypeptide variant composition prepared from the cells can be purified using, for example, hydroxyapatite chromatography, hydrophobic interaction chromatography, gel electrophoresis, dialysis, and affinity chromatography, with affinity chromatography being among one of the typically preferred purification steps. In some embodiments, an antigen binding polypeptide variant obtained according to a method herein comprises an epitope tag (e.g., a tag attached to the antigen binding polypeptide variant via a cleavable linker) to facilitate purification. Exemplary epitope tags include, but are not limited to, e.g., 6×His (also known as His-tag or hexahistidine tag), FLAG, HA, Myc, V5, GFP (green fluorescent protein, e.g., enhanced green fluorescent protein or EGFP), GST (glutathione-S-transferase), β-GAL (β-galactosidase), Luciferase, MBP (Maltose Binding Protein), RFP (Red Fluorescence Protein), and VSV-G (Vesicular Stomatitis Virus Glycoprotein.

### Harvesting or Recovering and Purifying Antibodies

In a related aspect, producing an antigen binding polypeptide variant obtained according to a method described herein comprises culturing a host cell described above under conditions that allow expression of the modified antigen binding polypeptide variant and recovering (such as harvesting) the antigen binding polypeptide variant. In certain embodiments, producing an antigen binding polypeptide variant made according to a method described herein further comprises purifying the recovered antigen binding polypeptide variant to obtain a preparation that is substantially homogeneous, e.g., for further assays and uses.

A antigen binding polypeptide variant made according to a method described herein can be produced intracellularly, or directly secreted into the medium. If such antigen binding polypeptide variant is produced intracellularly, as a first step, the particulate debris, either host cells or lysed fragments, are removed, for example, by centrifugation or ultrafiltration. Where the antigen binding polypeptide variant made according to a method described herein is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

Standard protein purification methods known in the art can be employed to obtain substantially homogeneous preparations of an antigen binding polypeptide variant made according to a method described herein from cells. The following procedures are exemplary of suitable purification procedures: fractionation on immunoaffinity or ion-exchange columns, ethanol precipitation, reverse phase HPLC, chromatography on silica or on a cation-exchange resin such as DEAE, chromatofocusing, SDS-PAGE, ammonium sulfate precipitation, and gel filtration using, for example, Sephadex G-75.

Additionally or alternatively, a modified antigen binding polypeptide variant made using a method described herein can be purified using, for example, hydroxyapatite chromatography, gel electrophoresis, dialysis, and affinity chromatography, with affinity chromatography being the preferred purification technique.

In certain aspects, wherein the antigen binding polypeptide variant comprises a human IgG region or portion thereof, the preparation derived from the cell culture medium as described above is applied onto a Protein A immobilized solid phase to allow specific binding of the antigen binding polypeptide variant to protein A. The solid phase is then washed to remove contaminants non-specifically bound to the solid phase. The antigen binding polypeptide variant is recovered from the solid phase by elution into a solution containing a chaotropic agent or mild detergent. Exemplary chaotropic agents and mild detergents include, but are not limited to, Guanidine-HCl, urea, lithium perclorate, arginine, histidine, SDS (sodium dodecyl sulfate), Tween, Triton, and NP-40, all of which are commercially available.

The suitability of protein A as an affinity ligand depends on the species and isotype of any immunoglobulin Fc domain that is present in the antigen binding polypeptide variant. Protein A can be used to purify antibodies that are based on human γ1, γ2, or γ4 heavy chains (Lindmark et al., J. Immunol. Meth. 62:1-13 (1983)). Protein G is recommended for all mouse isotypes and for human γ3 (Guss et al., EMBO J. 5:15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the modified antigen binding polypeptide variant comprises a C_{H}3 domain, the Bakerbond ABX™ resin (J. T. Baker, Phillipsburg, NJ) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antigen binding polypeptide variant to be recovered.

Following any preliminary purification step(s), the mixture comprising the antigen binding polypeptide variant and contaminants may be subjected to low pH hydrophobic interaction chromatography using an elution buffer at a pH between about 2.5-4.5, preferably performed at low salt concentrations (e.g., from about 0-0.25M salt). The production of a modified antigen binding polypeptide variant can alternatively or additionally (to any of the foregoing particular methods) comprise dialyzing a solution comprising a mixture of the polypeptides.

Exemplary Antibody Variable Domains with Reduced Viscosity

In some embodiments, provided are antibody variable domain variants produced using a method described herein. In some embodiments, the antibody variable domain variants are variants of an antibody heavy chain variable domain (V_{H}) set forth in SEQ ID NO: 1. In some embodiments, the V_{H} variant comprises an amino acid sequence set forth in any one of SEQ ID NOs:3-17 or 19-22. The amino acid sequences of SEQ ID Nos:1 and 3-17 and 19-22 are provided below. The variable domains are numbered following the numbering scheme of Kabat.

(SEQ ID NO: 1)
EVQLVESGGGLVQPGGSLRLSCAASGFNIYYNYIHWVRQAPGKGLEWVAEFSPYSGSTY
YADSVKGRFTISADTSKNTAYLQMNSLRAEDTAVYYCARSAAIVDWYDYFKGFDYWG
QGTLVTVSS
(SEQ ID NO: 3)
EVQLVESGGGLVQPGGSLRLSCAASGFNIYYNYIHWVRQAPGKGLEWVAEFSPYSGSTY
YADSVKGRFTISADTSKNTAYLQMNSLRAEDTAVYYCARSAAIKDWYDYFKGFDYWG
QGTLVTVSS
V99K
(SEQ ID NO: 4)
EVQLVESGGGLVQPGGSLRLSCAASGFNIYYNYIHWVRQAPGKGLEWVAEFSPYSGSTY
YADSVKGRFTISADTSKNTAYLQMNSLRAEDTAVYYCARSAAIVDFYDYFKGFDYWGQ
GTLVTVSS
W100aF
(SEQ ID NO: 5)
EVQLVESGGGLVQPGGSLRLSCAASGFNIYYNYIHWVRQAPGKGLEWVAEFSPYSGSTY
YADSVKGRFTISADTSKNTAYLQMNSLRAEDTAVYYCARSAAIVDVYDYFKGFDYWGQ
GTLVTVSS
W100aV
(SEQ ID NO: 6)
EVQLVESGGGLVQPGGSLRLSCAASGFNIYYNYIHWVRQAPGKGLEWVAEFSPYSGSTY
YADSVKGRFTISADTSKNTAYLQMNSLRAEDTAVYYCARSAAIVDLYDYFKGFDYWGQ
GTLVTVSS
W100aL
(SEQ ID NO: 7)
EVQLVESGGGLVQPGGSLRLSCAASGFNIYYNYIHWVRQAPGKGLEWVAEFSPYSGSTY
YADSVKGRFTISADTSKNTAYLQMNSLRAEDTAVYYCARSAAIVDAYDYFKGFDYWGQ
GTLVTVSS
W100aA
(SEQ ID NO: 8)
EVQLVESGGGLVQPGGSLRLSCAASGFNIYYNYIHWVRQAPGKGLEWVAEFSPYSGSTY
YADSVKGRFTISADTSKNTAYLQMNSLRAEDTAVYYCARSAAIVDWVDYFKGFDYWG
QGTLVTVSS
Y100bV
(SEQ ID NO: 9)
EVQLVESGGGLVQPGGSLRLSCAASGFNIYYNYIHWVRQAPGKGLEWVAEFSPYSGSTY
YADSVKGRFTISADTSKNTAYLQMNSLRAEDTAVYYCARSAAIVDWADYFKGFDYWG
QGTLVTVSS
Y100bA
(SEQ ID NO: 10)
EVQLVESGGGLVQPGGSLRLSCAASGFNIYYNYIHWVRQAPGKGLEWVAEFSPYSGSTY
YADSVKGRFTISADTSKNTAYLQMNSLRAEDTAVYYCARSAAIVDWHDYFKGFDYWG
QGTLVTVSS
Y100bH
(SEQ ID NO: 11)
EVQLVESGGGLVQPGGSLRLSCAASGFNIYYNYIHWVRQAPGKGLEWVAEFSPYSGSTY
YADSVKGRFTISADTSKNTAYLQMNSLRAEDTAVYYCARSAAIVDWKDYFKGFDYWG
QGTLVTVSS
Y100bK
(SEQ ID NO: 12)
EVQLVESGGGLVQPGGSLRLSCAASGFNIHYNYIHWVRQAPGKGLEWVAEFSPYSGSTY
YADSVKGRFTISADTSKNTAYLQMNSLRAEDTAVYYCARSAAIVDWYDYFKGFDYWG
QGTLVTVSS
Y30H
(SEQ ID NO: 13)
EVQLVESGGGLVQPGGSLRLSCAASGFNIYHNYIHWVRQAPGKGLEWVAEFSPYSGSTY
YADSVKGRFTISADTSKNTAYLQMNSLRAEDTAVYYCARSAAIVDWYDYFKGFDYWG
QGTLVTVSS
Y31H
(SEQ ID NO: 14)
EVQLVESGGGLVQPGGSLRLSCAASGFNIYYNYIHWVRQAPGKGLEWVAEFSPYRGSTY
YADSVKGRFTISADTSKNTAYLQMNSLRAEDTAVYYCARSAAIVDWYDYFKGFDYWG
QGTLVTVSS
S54R
(SEQ ID NO: 15)
EVQLVESGGGLVQPGGSLRLSCAASGFNIYYNYIHWVRQAPGKGLEWVAEFSPYSGRTY
YADSVKGRFTISADTSKNTAYLQMNSLRAEDTAVYYCARSAAIVDWYDYFKGFDYWG
QGTLVTVSS
S56R
(SEQ ID NO: 16)
EVQLVESGGGLVQPGGSLRLSCAASGFNIYYNYIHWVRQAPGKGLEWVAEFSPYSGSTY
YADSVKGRFTISADTSKNTAYLQMNSLRAEDTAVYYCARSAAIVDWQDYFKGFDYWG
QGTLVTVSS
Y100bQ
(SEQ ID NO: 17)
EVQLVESGGGLVQPGGSLRLSCAASGFNIYYNYIHWVRQAPGKGLEWVAEFSPYSGSTY
YADSVKGRFTISADTSKNTAYLQMNSLRAEDTAVYYCARSAAIVDWRDYFKGFDYWG
QGTLVTVSS
Y100bR
(SEQ ID NO: 19)
EVQLVESGGGLVQPGGSLRLSCAASGFNIYYNYIHWVRQAPGKGLEWVAEFSPYSG
STYYADSVKGRFTISADTSKNTAYLQMNSLRAEDTAVYYCARSAAIVDHYDYFKGF
DYWGQGTLVTVSS
W100aH
(SEQ ID NO: 20)
EVQLVESGGGLVQPGGSLRLSCAASGFNIYYNYIHWVRQAPGKGLEWVAEFSPYSG
STYYADSVKGRFTISADTSKNTAYLQMNSLRAEDTAVYYCARSAAIVDKYDYFKGF
DYWGQGTLVTVSS
W100aK
(SEQ ID NO: 21)
EVQLVESGGGLVQPGGSLRLSCAASGFNIYYNYIHWVRQAPGKGLEWVAEFSPYSG
STYYADSVKGRFTISADTSKNTAYLQMNSLRAEDTAVYYCARSAAIVDRYDYFKGF
DYWGQGTLVTVSS
W100aR
(SEQ ID NO: 22)
EVQLVESGGGLVQPGGSLRLSCAASGFNIYYNYIHWVRQAPGKGLEWVAEFSPYSG
STYYADSVKGRFTISADTSKNTAYLQMNSLRAEDTAVYYCARSAAIVDWHDYFKGF
DYWGQGTLVTVSS
Y100bH

In some embodiments, provided is an antibody comprising a V_{H} set forth in any one of SEQ ID NOs: 3-17 or 19-22 and an antibody light chain variable domain (V_{L}) set forth in SEQ ID NO:2. In some embodiments, provided is an antibody comprising a V_{H} set forth in any one of SEQ ID NOs: 3-17 or 19-22 and an antibody light chain variable domain (V_{L}) set forth in SEQ ID NO: 18.

As shown in the Examples, antibodies comprising a V_{H} set forth in any one of SEQ ID NOs:3-17 or 19-22 and a V_{L} set forth in SEQ ID NO:2 or 18 exhibit lower viscosity as compared to antibodies comprising a V_{H} Set forth in SEQ ID NO:1 and a V_{L} set forth in SEQ ID NO:2. The variable domains are numbered following the numbering scheme of Kabat. The amino acid sequence of SEQ ID NO:2 is provided below:

(SEQ ID NO: 2)
DIQMTQSPSSLSASVGDRVTITCRASQSVSSAVAWYQQKPGKAPKLLIYSA
SSLYSGVPSRFSGSRSGTDFTLTISSLQPEDFATYYCQQYGSYLYTFGQGT
KVEIK.

In some embodiments, the antibody variable domain variants are variants of an antibody light chain variable domain (V_{L}) set forth in SEQ ID NO:2. In some embodiments, the V_{L} variant comprises an amino acid sequence set forth in any one of SEQ ID NO: 18. The amino acid sequences of SEQ ID NO: 18 is provided below.

(SEQ ID NO: 18)
DIQMTQSPSSLSASVGDRVTITCRASQSVSSAVAWYQQKPGKAPKLLIY
SASSLYSGVPSRFSGSRSGTDFTLTISSLQPEDFATYYCQQYGSKLYTF
GQGTKVEIK
Y94K

As shown in the Examples, antibodies comprising a V_{L} set forth in SEQ ID NO:18 exhibits lower viscosity as compared to antibodies comprising a V_{L} set forth in SEQ ID NO:2. In some embodiments, provided is an antibody comprising an antibody light chain variable domain (V_{L}) set forth in SEQ ID NO:18 and a V_{H} set forth in any one of SEQ ID NOs:1, 3-17, or 19-22.

All publications and patent applications cited in this specification are herein incorporated by reference as if each individual publication or patent application were specifically and individually indicated to be incorporated by reference.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1A: Deep Mutational Analysis of the Variable Domains of an Anti-Glucagon Receptor (GCGR) Antibody

The following experiments were performed using an anti-GCGR (G protein-coupled glucagon receptor) IgG1 antibody comprising the V_{H} domain set forth in SEQ ID NO:1 and the V_{L} domain set forth in SEQ ID NO:2 set as an exemplary antibody that exhibits elevated viscosity at high concentrations. SEQ ID NOs:1 and 2 are provided below. Further details regarding this anti-GCGR antibody are provided in WO 2013/059531, the contents of which are incorporated by reference herein in their entirety.

(SEQ ID NO: 1)
EVQLVESGGGLVQPGGSLRLSCAASGFNIYYNYIHWVRQAPGKGLEWV
AEFSPYSGSTYYADSVKGRFTISADTSKNTAYLQMNSLRAEDTAVYYC
ARSAAIVDWYDYFKGFDYWGQGTLVTVSS
(SEQ ID NO: 2)
DIQMTQSPSSLSASVGDRVTITCRASQSVSSAVAWYQQKPGKAPKLL
IYSASSLYSGVPSRFSGSRSGTDFTLTISSLQPEDFATYYCQQYGSY
LYTFGQGTKVEIK

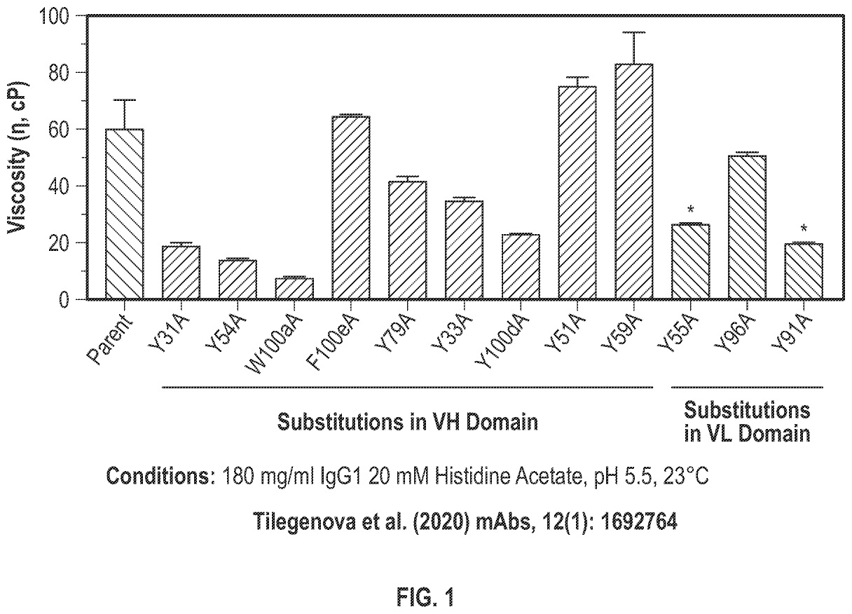

The CDRs of antibodies are commonly enriched in aromatic residues that contribute to antigen-binding affinity (Traxlmayr et al. (2016) J Biol Chem, 291:22496-508). In previous studies (see Tilegenova et al. (2020) mAbs, 12(1): 1692764) 16 aromatic residues within the variable regions of the anti-GCGR IgG1 were mutated to alanine to investigate a possible role for aromatic residues in the high viscosity of this antibody. Twelve of the 16 mutants were expressed in sufficient quantities to be used for further studies. See FIG. 1. Viscosity was measured at 180 mg/mL final concentration in 20 mM histidine-acetate buffer at pH 5.5. Seven different alanine variants were identified that substantially lowered viscosity (~2 to ~8-fold reduction). See FIG. 1. This result suggests the general involvement of aromatic amino acid residues in the intermolecular interactions underlying the high viscosity displayed by concentrated antibody solutions. Two of these seven variants, namely V_{L} Y55A and Y91A, also retained antigen-binding affinity for GCGR, a desirable attribute for a potential antibody clinical candidate. See Table 1. Antigen binding affinities (K_{D} values) were assessed via surface plasmon resonance.

TABLE 1

| Variant | K_{D} (nM) |
|---|---|
| Parent | 0.61 |
| V_{H} Y59A | 0.32 |
| V_{L} Y55A* | 0.74 |
| V_{L} Y91A* | 0.84 |
| V_{L} Y96A | 0.93 |
| V_{H} F51A | 1.5 |
| V_{H} Y79A | 1.8 |
| V_{H} Y100dA | 5.0 |
| V_{H} W100aA | 11 |
| V_{H} F100eA | 14 |
| V_{H} Y54A | 46 |
| V_{H} Y33A | 53 |
| V_{H} Y31A | 206 |

*Antibody variants that exhibited reduced viscosity by >2-fold relative to parental antibody and maintained K_{D} within 2-fold of the parent antibody.

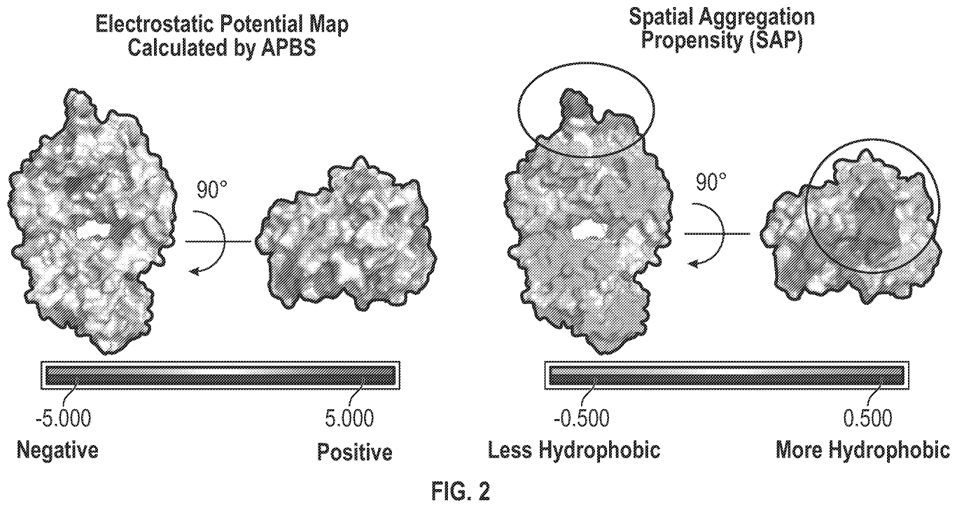

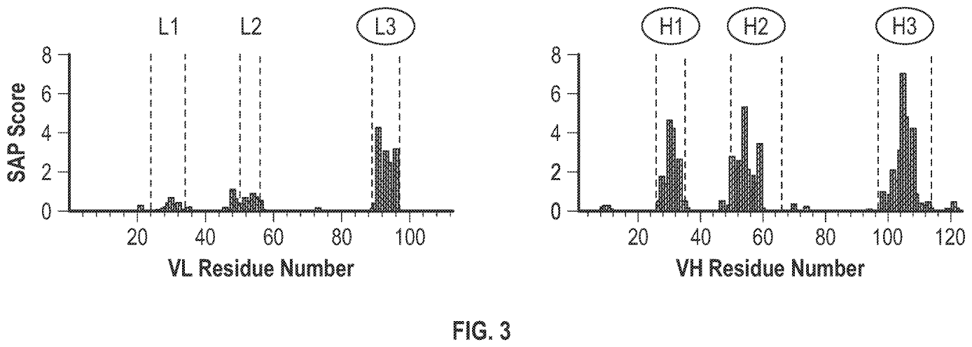

As shown in FIG. 1, not all alanine substitution variants exhibited reduced viscosity, suggesting that other types of intermolecular interactions may also contribute to high viscosity. To investigate such molecular interactions, the crystal structure of the anti-GCGR Fab was solved (2.7 Å resolution) and surface analyses were performed to locate charged and/or hydrophobic patches on the Fab. Briefly, an electrostatic potential map of the Fab was calculated by Adaptive Poisson-Boltzmann Solver (APBS, see, e.g., www(dot)cgl(dot)ucsf(dot)edu/chimera/docs/ContributedSoftware/apbs/apbs(dot)html and Baker et al. (2001) PNAS USA, 98(18): 10037-10041). See FIG. 2, left side. The spatial aggregation propensity (SAP) of the Fab was also determined, e.g., as described in Chennamsetty et al. (2009) PNAS USA, 106(29): 11937-11942 and Lauer et al. (2012) J Pharm Sci, 101(1): 102-115. See FIG. 2, right side. Molecular dynamic (MD) simulation was incorporated to prepare an averaged model for SAP considering the flexibility of antibody molecules in solution. See, e.g., R. Salomon-Ferrer, D. A. Case, R. C. Walker. (2013) “An overview of the Amber biomolecular simulation package.” WIREs Comput. Mol. Sci. 3, 198-210; and D. A. Case, T. E. Cheatham, III, T. Darden, H. Gohlke, R. Luo, K. M. Merz, Jr., A. Onufriev, C. Simmerling, B. Wang and R. Woods. (2005) “The Amber biomolecular simulation programs.” J Computat. Chem. 26, 1668-1688. A hydrophobic patch on the anti-GCGR Fab was identified (circled in FIG. 2, right side). Further, four high-SAP regions (e.g., having a SAP value of ≥2, such as 2.3 or greater) were identified within the CDRs of the anti-GCGR Fab. See FIG. 3. Of the 6 CDRs illustrated, 4 CDRs (L3, H1, H2 and H3) have multiple (but not all) residues with SAP values >2.

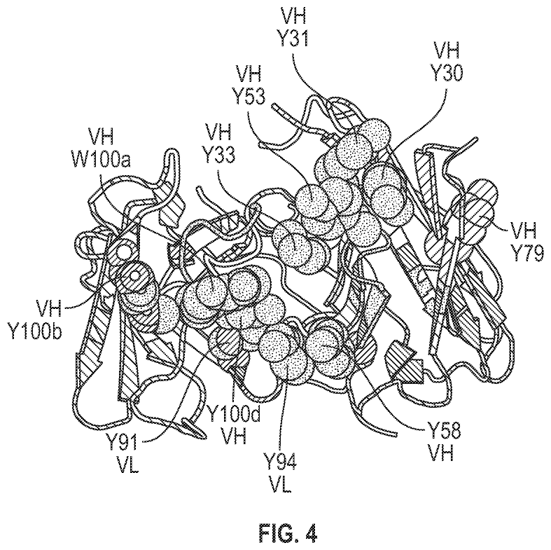

Applicant hypothesized that disruption of hydrophobic patches within the CDRs would reduce viscosity. To test this hypothesis, substitution variants were generated in which one or more aromatic residues in the hydrophobic patches in the CDR-H1, CDR-H2, CDR-H3, and CDR-L3 were replaced with alanines. FIG. 4 shows the aromatic residues in the CDRs of the anti-GCGR Fab that were substituted.

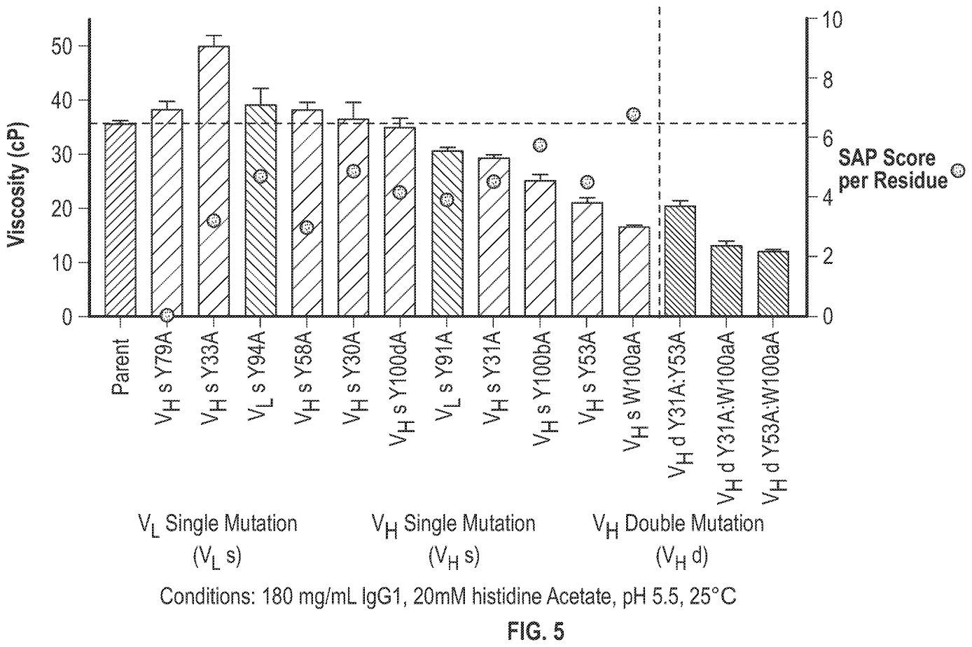

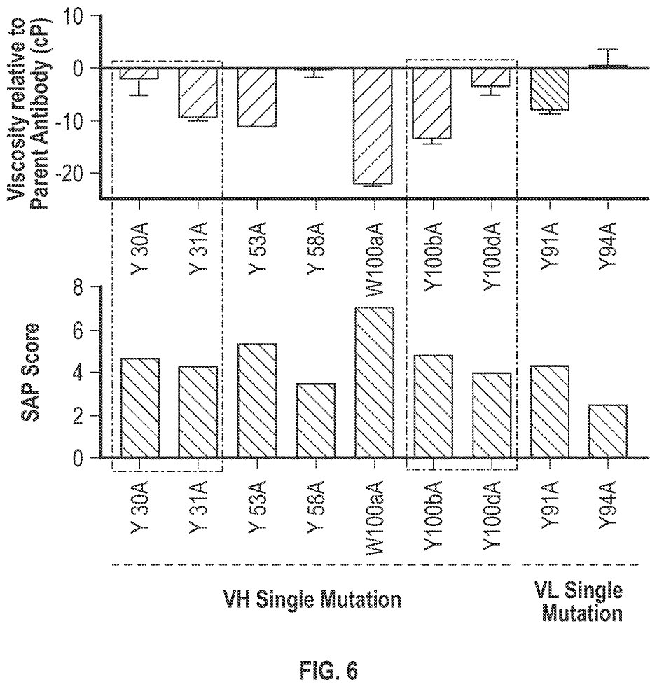

FIG. 5 shows the effect of single substitutions in the V_{H} and V_{L} and double substitutions in the VH on viscosity at 180 mg/ml IgG1, 20 mM histidine acetate, pH 5.5, 25° C. A comparison of the viscosity of single substitution variant IgG1 antibodies shown in FIG. 5 relative to the parent IgG1 antibody vs. SAP value of the original wild type aromatic residue at each position is shown in FIG. 6. Consistent with results reported in Tilegenova et al. (2020) mAbs, 12(1): 1692764, not all alanine substitutions decreased viscosity. For example, the Y31A substitution reduced viscosity of the variant relative to the parental antibody, whereas the Y30A substitution, which neighbors the Y31A substitution, did not reduce the viscosity of the variant relative to the parental antibody. Similarly, the Y100bA substitution reduced viscosity of the variant relative to the parental antibody, whereas the Y100dA substitution, which neighbors, the Y100bA substitution, did not. Such results suggest that SAP value alone is not sufficient to predict key viscosity-affecting residues.

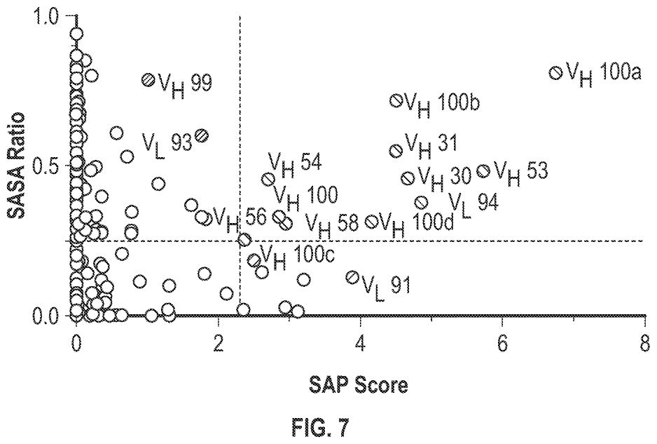

Next, the solvent accessible surface area (SASA) ratios for each of the amino acids identified as having high SAP values (e.g., SAP values ≥2, such as 2.3 or greater) were calculated, e.g., as described at ssbio(www)readthedocs(dot)io/en/latest/instructions/msms(dot)html. In brief, the SASA ratio is calculated by dividing the surface accessible area of an amino acid side chain by the total surface area of the side chain. FIG. 7 shows the SASA ratios of certain amino acid positions in the CDRs of the anti-GCGR IgG1 antibody, including those residues having SAP values ≥2. The dotted lines in FIG. 7 indicate cutoff values used for high SASA ratio (0.25) and high SAP score (2.3). Amino acids having high SAP values and high SASA, such as Y31, Y100b, Y53, and Y100a in the VH domain of the anti-GCGR antibody, are predicted to play a significant role in the viscosity of the antibody and are predicted play a role in mitigating viscosity when substituted. Additionally, representative sites in the high SASA ratio and low SAP region (labeled residues in the upper left quadrant) as well as in the low SASA ratio and high SAP region (labeled residues in the lower right quadrant) were also included to provide a more comprehensive analysis. The other amino acid positions shown as unlabeled circles in FIG. 7 are not predicted to affect viscosity, e.g., when mutated to alanine.

Applicant then constructed ~200 antibody single mutant antibody variants in which amino acids in the CDRs determined to have high SAP values (e.g., ≥2, such as 2.3 or greater) and high SASA ratios (e.g., ≥0.25 or ≥0.5) were substituted. (Gly, Pro, Cys, and Met substitution variants were not constructed.) The antibody variants were first screened via dynamic light scattering (DLS) at concentrations between about 2 mg/ml and about 10 mg/ml to measure each variant's interaction parameter using the following equation:

$$D = {D}_{0} ( 1 + {k}_{D} * c )$$

where D=translational diffusion coefficient; Do is the self-diffusion coefficient (the value of D at zero concentration); c=protein concentration; and k_{D}=interaction parameter. The more positive a variant's k_{D} value, the weaker the variant's self-interaction. Conversely, the more negative a variant's k_{D} value, the stronger the variant's self-interaction. See, e.g., Sorret et al. (2016) Biophysical Journal, 111: 1831-1842.

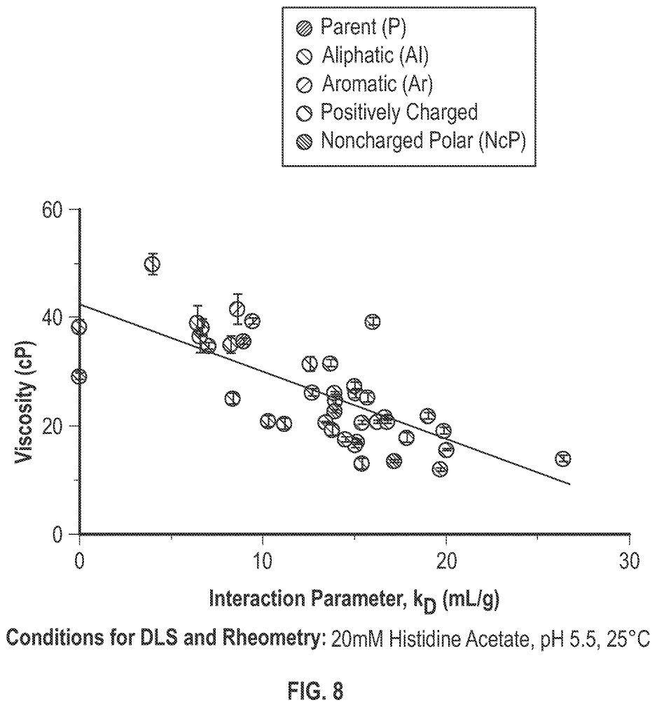

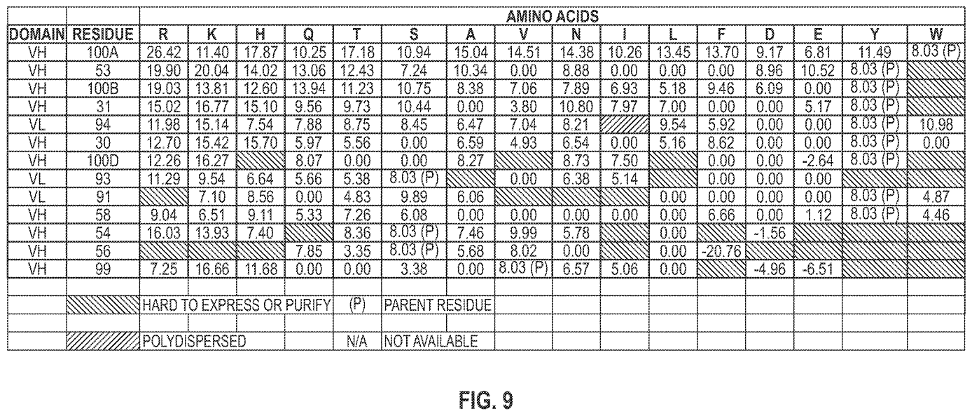

FIG. 9 provides a table showing the effect of certain replacement amino acid of the anti-GCGR IgG1 antibody on DLS interaction parameter (ml/g). Shown are DLS interaction parameters measured at 25.0° C. in 20 mM histidine acetate, pH 5.5. Variants with exceptionally low diffusion coefficient are highlighted (grey), indicating their polydispersed behavior in solution. The parental residues are highlighted using (P). Mutants with expression or purification issues or exhibited significant aggregation are also highlighted (black). A subset of anti-GCGR IgG₁ variants from this DLS study (FIG. 9) were assessed for viscosity at high concentration. Specifically, ~30 variants were selected spanning a broad range of interaction parameter values (0-26 mL/g) comparable, above or below that of the parent antibody (8 mL/g). These variants represent diverse replacement amino acid types: aliphatic, aromatic, positively charge and noncharged polar. Variants with a negative DLS interaction parameter were additionally planned for this study but were lost due to precipitation during the spin concentration process and were therefore excluded from viscosity measurements. Additionally included were ~10 single alanine substitution variants from FIG. 5. FIG. 8 shows a plot of DLS interaction parameter (k_{D}, ml/g) and viscosity (η, cP) for these exemplary anti-GCGR IgG1 antibody variants. The plot suggests that there is a correlation between DLS interaction parameter and viscosity of anti-GCGR antibody variants. DLS interaction parameter values that are lower than the wild type value correlate with higher viscosity, whereas DLS interaction parameter values that are higher than the wild type value correlate with lower viscosity.

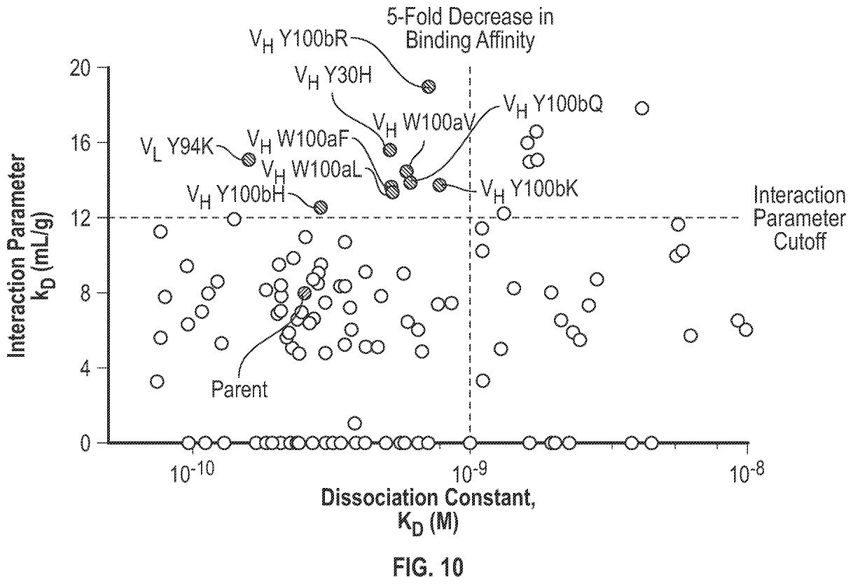

FIG. 10 shows a plot of DLS interaction parameter (k_{D}, ml/g) for each the anti-GCGR IgG1 antibody variants tested in FIG. 9 and each variant's dissociation constant (K_{D}, M). Some variants, namely, variants comprising a VH domain that comprises a W100aF, Y30H, W100aV, W100aL, Y100bK, Y100bH, Y100bR, or Y100bQ substitution and a VL domain set forth in SEQ ID NO:2 or a VL domain substitution of Y94K showed increased DLS interaction parameter with little effect on antigen-binding affinity (e.g. less than a 5-fold decrease in binding affinity as compared to the parent). See the top left quadrant of FIG. 10.

Provided in Table 2 is additional specific information of the variants of the top left quadrant of FIG. 10.

TABLE 2
Characterization data of certain variants provided herein.

| Anti-GCGR | | | | | | |
|---|---|---|---|---|---|---|
| IgG₁ | Interaction | | | | IgG | |
| antibody | parameter, | kₒₙ | k_{off} | K_{D} | monomer | Viscosity |
| variants | k_{D} (mL g⁻¹) | (M⁻¹ s⁻¹) | (s⁻¹) | (M) | (%) | (cP) |
| Parent | 8.03 | 1.92E+06 | 4.86E−04 | 2.53E−09 | 96.7 | 35.6 |
| VH Y100bH | 12.60 | 1.25E+06 | 3.62E−04 | 2.90E−09 | 100.0 | 31.6 |
| VH Y100bR | 19.03 | 6.79E+05 | 4.81E−04 | 7.08E−09 | 100.0 | 22.0 |
| VH W100aL | 13.45 | 5.67E+06 | 2.97E−03 | 5.24E−09 | 100.0 | 20.7 |
| VH W100aF | 13.70 | 2.13E+06 | 1.11E−03 | 5.20E−09 | 100.0 | 31.6 |
| VH Y30H | 15.70 | 2.22E+06 | 1.14E−03 | 5.14E−09 | 99.9 | 25.4 |
| VH Y100bQ | 13.94 | 1.54E+06 | 9.38E−04 | 6.08E−09 | 100.0 | 22.7 |
| VH W100aV | 14.51 | 4.33E+06 | 2.54E−03 | 5.88E−09 | 100.0 | 17.6 |
| VH Y100bK | 13.81 | 7.38E+05 | 5.72E−04 | 7.75E−09 | 100.0 | 19.3 |
| VL Y94K | 15.14 | 3.31E+06 | 5.31E−04 | 1.60E−09 | 99.1 | 17.1 |

Multiple additional parameters were tested for the parent and variants. Specifically, computational developability analysis was conducted using the therapeutic antibody profiler (TAP). The amber TAP flag identified for the VH W100aL variant was for surface hydrophobicity. Also shown are the calculated isoelectric points for the Fv fragment, polyspecificity from the baculovirus ELISA and predicted immunogenic T cell epitopes using NetMHCIIpan-4.0 EL. The humanness score was determined from the OASis percentile. Results are provided in Tables 3A and 3B. Specifically, Table 3A-3B provide characterization data of tested anti-GCGR IgG1 antibody variants described herein. Experimental data shown include the dynamic light scattering interaction parameter (k D), antigen-binding kinetics (kon, koff and KD) measured by surface plasmon resonance, percentage IgG1 monomer determined by analytical size exclusion chromatography and viscosity measured by cone-and-plate rheometry at 180 mg/mL IgG1 at 25.0° in 20 mM histidine acetate, pH 5.5. Computational data shown include the TAP scores and flags, isoelectric points and SAP values. nd, binding not detected; nm, not measured.

TABLE 3A
Multi-parameter evaluation of parent and varient antibodies.

| Anti-GCGR | Experimental data | | | | | | | | | | |
|---|---|---|---|---|---|---|---|---|---|---|---|
| IgG₁ | Interaction | | | | IgG₁ | | | | | | |
| antibody | parameter, | kₒₙ | k_{off} | K_{D} | monomer | Viscosity | TAP scores | | | | |
| variants | k_{D} (mL g⁻¹) | (M⁻¹ s⁻¹) | (s⁻¹) | (M) | (%) | (cP) | CDRL | PSH | PPC | PNC | SFvCSP |
| Parent | 8.03 | 1.92E+06 | 4.86E−04 | 2.53E−09 | 96.7 | 35.6 | 52 | 159.60 | 0.04 | 0.39 | 4.00 |
| VH S54A | 7.46 | 1.10E+06 | 8.45E−04 | 7.65E−09 | 100.0 | nm | 52 | 154.92 | 0.04 | 0.39 | 4.00 |
| VH S54D | −1.56 | 1.33E+06 | 2.58E−02 | 1.94E−07 | 100.0 | nm | 52 | 158.88 | 0.04 | 0.39 | 0.00 |
| VH S54H | 7.40 | 1.90E+07 | 5.11E−02 | 2.68E−08 | 98.1 | nm | 52 | 141.20 | 0.04 | 0.39 | 4.40 |
| VH S54K | 13.93 | 6.87E+05 | 1.00E−02 | 1.46E−07 | 99.1 | 26.1 | 52 | 157.60 | 0.04 | 0.39 | 8.00 |
| VH S54L | 0.00 | 1.93E+06 | 3.78E−03 | 1.96E−08 | 98.3 | nm | 52 | 161.58 | 0.04 | 0.39 | 4.00 |
| VH S54N | 5.78 | 1.13E+07 | 7.02E−02 | 6.20E−08 | 98.4 | nm | 52 | 147.43 | 0.04 | 0.39 | 4.00 |
| VH S54R | 16.03 | 1.82E+06 | 2.92E−03 | 1.60E−08 | 100.0 | 39.3 | 52 | 147.47 | 0.04 | 0.39 | 8.00 |
| VH S54T | 8.36 | 2.02E+06 | 6.87E−04 | 3.40E−09 | 100.0 | nm | 52 | 146.32 | 0.04 | 0.39 | 4.00 |
| VH S54V | 9.99 | 3.79E+05 | 2.10E−03 | 5.54E−08 | 100.0 | nm | 52 | 148.67 | 0.04 | 0.39 | 4.00 |
| VH S54A | 5.68 | 4.51E+06 | 3.46E−04 | 7.68E−10 | 100.0 | nm | 52 | 151.80 | 0.04 | 0.18 | 8.00 |
| VH S56F | −20.76 | 1.63E+06 | 2.68E−04 | 1.64E−09 | 100.0 | nm | 52 | 152.36 | 0.04 | 0.39 | 4.00 |
| VH S56L | 0.00 | 1.78E+06 | 3.39E−04 | 1.90E−09 | 100.0 | nm | 52 | 156.09 | 0.04 | 0.39 | 4.00 |
| VH S56N | 0.00 | 2.51E+06 | 1.04E−03 | 4.14E−09 | 95.2 | nm | 52 | 149.47 | 0.04 | 0.39 | 4.00 |
| VH S56Q | 7.85 | 2.24E+06 | 1.78E−04 | 7.94E−10 | 99.3 | nm | 52 | 147.14 | 0.04 | 0.39 | 4.00 |
| VH S56T | 3.35 | 1.74E+06 | 3.54E−04 | 7.48E−10 | 99.1 | nm | 52 | 152.09 | 0.04 | 0.39 | 4.00 |
| VH S56V | 8.02 | 1.96E+06 | 2.24E−04 | 1.14E−09 | 100.0 | nm | 52 | 165.51 | 0.04 | 0.39 | 4.00 |
| VH V99A | 0.00 | 1.53E+06 | 2.50E−03 | 1.64E−08 | 100.0 | nm | 52 | 153.40 | 0.04 | 0.39 | 4.00 |
| VH V99D | −4.96 | 1.71E+06 | 3.45E−03 | 2.01E−08 | 99.7 | nm | 52 | 141.38 | 0.04 | 1.58 | 0.00 |
| VH V99E | −6.51 | nd | nd | nd | 99.6 | nm | 52 | 157.13 | 0.04 | 1.58 | 0.00 |
| VH V99H | 11.68 | 1.55E+06 | 8.70E−03 | 5.62E−08 | 100.0 | nm | 52 | 141.21 | 0.04 | 0.39 | 4.40 |
| VH V99I | 5.06 | 2.30E+06 | 2.96E−03 | 1.29E−08 | 100.0 | nm | 52 | 173.54 | 0.04 | 0.22 | 4.00 |
| VH V99K | 16.66 | 2.21E+06 | 3.81E−03 | 1.73E−08 | 99.9 | 21.7 | 52 | 157.07 | 0.04 | 0.39 | 8.00 |
| VH V99L | 0.00 | 2.50E+06 | 5.71E−03 | 2.28E−08 | 100.0 | nm | 52 | 159.66 | 0.04 | 0.39 | 4.00 |
| VH V99N | 6.57 | 1.47E+06 | 1.35E−02 | 9.16E−08 | 100.0 | nm | 52 | 157.38 | 0.04 | 0.39 | 4.00 |
| VH V99Q | 0.00 | 2.68E+05 | 1.16E−02 | 4.33E−07 | 99.9 | nm | 52 | 147.08 | 0.04 | 0.39 | 4.00 |
| VH V99R | 7.25 | 4.77E+04 | 7.17E−03 | 1.50E−06 | 99.9 | nm | 52 | 146.97 | 0.04 | 0.39 | 8.00 |
| VH V99S | 3.38 | 2.81E+06 | 3.11E−03 | 1.11E−08 | 99.9 | nm | 52 | 158.02 | 0.04 | 0.39 | 4.00 |
| VH V99T | 0.00 | 2.90E+06 | 1.69E−03 | 5.82E−09 | 96.4 | nm | 52 | 158.16 | 0.04 | 0.39 | 4.00 |
| VH W100aA | 15.04 | 1.55E+06 | 2.52E−03 | 1.63E−08 | 99.5 | 16.6 | 52 | 173.73 | 0.04 | 0.22 | 4.00 |
| VH W100aD | 9.17 | nd | nd | nd | 99.3 | nm | 52 | 171.77 | 0.04 | 1.48 | 0.00 |
| VH W100aE | 6.81 | nd | nd | nd | 99.2 | nm | 52 | 147.42 | 0.04 | 1.39 | 4.00 |
| VH W100aF | 13.70 | 2.13E+06 | 1.11E−03 | 5.20E−09 | 100.0 | 31.6 | 52 | 163.60 | 0.04 | 0.39 | 4.00 |
| VH W100aH | 17.87 | 1.63E+06 | 6.78E−03 | 4.17E−08 | 99.6 | 17.9 | 52 | 152.14 | 0.04 | 0.18 | 8.40 |
| VH W100aI | 10.26 | 8.71E+05 | 9.68E−04 | 1.11E−08 | 100.0 | nm | 52 | 154.90 | 0.04 | 0.18 | 8.00 |
| VH W100aK | 11.40 | nd | nd | nd | 99.4 | nm | 52 | 147.40 | 0.04 | 0.18 | 12.00 |
| VH W100aL | 13.45 | 5.67E+06 | 2.97E−03 | 5.24E−09 | 100.0 | 20.7 | 52 | 174.83 | 0.04 | 0.22 | 4.00 |
| VH W100aN | 14.38 | nd | nd | nd | 99.5 | nm | 52 | 171.78 | 0.04 | 0.22 | 4.00 |
| VH W100aQ | 10.25 | 1.18E+06 | 6.86E−03 | 5.83E−08 | 99.7 | nm | 52 | 147.41 | 0.04 | 0.18 | 8.00 |
| VH W100aR | 26.42 | nd | nd | nd | 100.0 | 14.1 | 52 | 147.34 | 0.04 | 0.18 | 12.00 |
| VH W100aS | 10.94 | 1.15E+06 | 1.88E−02 | 1.63E−07 | 99.5 | nm | 52 | 153.17 | 0.00 | 0.37 | 4.00 |
| VH W100aT | 17.18 | 9.61E+05 | 1.11E−02 | 1.16E−07 | 100.0 | 13.7 | 52 | 153.21 | 0.00 | 0.37 | 4.00 |
| VH W100aV | 14.51 | 4.33E+06 | 2.54E−03 | 5.88E−09 | 100.0 | 17.6 | 52 | 154.55 | 0.04 | 0.18 | 8.00 |
| VH W100aY | 11.49 | 1.37E+06 | 1.50E−03 | 1.10E−08 | 99.6 | nm | 52 | 166.25 | 0.04 | 0.39 | 4.00 |
| VH Y100bA | 8.38 | 1.74E+06 | 6.17E−04 | 3.54E−09 | 99.7 | 25.2 | 52 | 152.30 | 0.00 | 0.11 | 12.00 |
| VH Y100bD | 6.09 | 2.10E+06 | 1.36E−03 | 6.48E−09 | 99.5 | nm | 52 | 152.34 | 0.00 | 1.72 | 0.00 |
| VH Y100bE | 0.00 | 5.02E+06 | 1.05E−03 | 2.08E−09 | 99.4 | nm | 52 | 156.95 | 0.00 | 1.72 | 4.00 |
| VH Y100bF | 9.46 | 7.41E+06 | 7.10E−04 | 9.58E−10 | 100.0 | 39.3 | 52 | 154.79 | 0.04 | 0.18 | 8.00 |
| VH Y100bH | 12.60 | 1.25E+06 | 3.62E−04 | 2.90E−09 | 100.0 | 31.6 | 52 | 156.11 | 0.00 | 0.19 | 8.40 |
| VH Y100bI | 6.93 | 4.35E+06 | 8.77E−04 | 2.02E−09 | 100.0 | nm | 52 | 159.26 | 0.00 | 0.19 | 8.00 |
| VH Y100bK | 13.81 | 7.38E+05 | 5.72E−04 | 7.75E−09 | 100.0 | 19.3 | 52 | 155.77 | 0.00 | 0.19 | 12.00 |
| VH Y100bL | 5.18 | 1.84E+06 | 7.75E−04 | 4.21E−09 | 100.0 | nm | 52 | 158.98 | 0.00 | 0.19 | 8.00 |
| VH Y100bN | 7.89 | 1.25E+06 | 5.97E−04 | 4.78E−09 | 99.6 | nm | 52 | 150.36 | 0.00 | 0.11 | 12.00 |
| VH Y100bQ | 13.94 | 1.54E+06 | 9.38E−04 | 6.08E−09 | 100.0 | 22.7 | 52 | 150.36 | 0.00 | 0.11 | 12.00 |
| VH Y100bR | 19.03 | 6.79E+05 | 4.81E−04 | 7.08E−09 | 100.0 | 22.0 | 52 | 150.03 | 0.00 | 0.11 | 16.00 |
| VH Y100bS | 10.75 | 1.46E+06 | 5.17E−04 | 3.55E−09 | 99.7 | nm | 52 | 157.01 | 0.00 | 0.19 | 8.00 |
| VH Y100bT | 11.23 | 1.14E+06 | 6.19E−04 | 5.44E−09 | 99.7 | nm | 52 | 151.12 | 0.00 | 0.11 | 12.00 |
| VH Y100bV | 7.06 | 4.63E+06 | 4.99E−04 | 1.08E−09 | 100.0 | 34.7 | 52 | 159.13 | 0.00 | 0.19 | 8.00 |
| VH Y100bA | 8.27 | 1.69E+06 | 2.43E−03 | 1.44E−08 | 94.9 | 35.0 | 52 | 165.20 | 0.00 | 0.19 | 8.00 |
| VH Y100bD | 0.00 | nd | nd | nd | 99.9 | nm | 52 | 162.57 | 0.00 | 1.32 | 4.00 |
| VH Y100bE | −2.64 | 8.38E+06 | 5.15E−02 | 6.14E−08 | 100.0 | nm | 52 | 157.78 | 0.00 | 1.32 | 4.00 |
| VH Y100bF | 0.00 | 1.64E+06 | 8.17E−04 | 4.99E−09 | 97.4 | nm | 52 | 176.77 | 0.04 | 0.22 | 4.00 |
| VH Y100bI | 7.50 | 2.13E+06 | 1.82E−03 | 8.54E−09 | 97.3 | nm | 52 | 160.40 | 0.00 | 0.19 | 8.00 |
| VH Y100bK | 16.27 | 2.00E+04 | 3.69E−03 | 1.84E−06 | 100.0 | 20.8 | 52 | 156.36 | 0.27 | 0.19 | 12.00 |
| VH Y100bN | 8.73 | 1.57E+06 | 4.50E−03 | 2.86E−08 | 100.0 | nm | 52 | 162.59 | 0.00 | 0.19 | 8.00 |
| VH Y100bQ | 8.07 | 1.92E+06 | 3.74E−03 | 1.95E−08 | 97.7 | nm | 52 | 163.15 | 0.00 | 0.19 | 8.00 |
| VH Y100bR | 12.26 | 1.95E+06 | 2.57E−03 | 1.32E−08 | 92.5 | nm | 52 | 153.72 | 0.20 | 0.19 | 8.00 |
| VH Y100bS | 0.00 | 2.54E+06 | 2.52E−03 | 9.92E−09 | 96.5 | nm | 52 | 163.64 | 0.00 | 0.19 | 8.00 |
| VH Y100bT | 0.00 | 2.06E+05 | 4.09E−03 | 1.98E−07 | 98.2 | nm | 52 | 156.97 | 0.00 | 0.19 | 8.00 |
| VH Y30A | 6.59 | 1.01E+06 | 2.16E−03 | 2.13E−08 | 99.9 | 36.5 | 52 | 161.03 | 0.04 | 0.39 | 4.00 |
| VH Y30D | 0.00 | 2.89E+06 | 5.83E−03 | 2.02E−08 | 100.0 | nm | 52 | 158.57 | 0.04 | 0.39 | 0.00 |
| VH Y30E | 0.00 | 6.58E+05 | 2.97E−03 | 4.51E−08 | 100.0 | nm | 52 | 147.62 | 0.04 | 0.39 | 0.00 |
| VH Y30F | 8.62 | 4.34E+06 | 5.33E−04 | 1.23E−09 | 100.0 | 41.5 | 52 | 161.93 | 0.04 | 0.39 | 4.00 |
| VH Y30H | 15.70 | 2.22E+06 | 1.14E−03 | 5.14E−09 | 99.9 | 25.4 | 52 | 159.06 | 0.04 | 0.39 | 4.40 |
| VH Y30I | 0.00 | 2.00E+06 | 1.11E−03 | 5.54E−09 | 100.0 | nm | 52 | 138.05 | 0.00 | 0.11 | 12.00 |
| VH Y30K | 15.42 | 5.86E+04 | 2.13E−03 | 3.63E−07 | 100.0 | 20.7 | 52 | 147.42 | 0.04 | 0.39 | 8.00 |
| VH Y30L | 5.16 | 1.36E+06 | 6.29E−04 | 4.63E−09 | 100.0 | nm | 52 | 152.83 | 0.04 | 0.39 | 4.00 |
| VH Y30N | 6.54 | 3.15E+05 | 3.25E−03 | 1.03E−07 | 100.0 | nm | 52 | 148.52 | 0.04 | 0.39 | 4.00 |
| VH Y30Q | 5.97 | 8.89E+05 | 2.08E−03 | 2.34E−08 | 100.0 | nm | 52 | 158.47 | 0.04 | 0.39 | 4.00 |
| VH Y30R | 12.70 | 2.03E+05 | 2.24E−03 | 1.10E−07 | 100.0 | 26.3 | 52 | 147.83 | 0.04 | 0.39 | 8.00 |
| VH Y30S | 0.00 | 2.33E+05 | 6.09E−03 | 2.61E−07 | 100.0 | nm | 52 | 149.79 | 0.04 | 0.39 | 4.00 |
| VH Y30T | 5.56 | 7.93E+05 | 1.96E−03 | 2.48E−08 | 99.5 | nm | 52 | 148.84 | 0.04 | 0.39 | 4.00 |
| VH Y30V | 4.93 | 1.24E+06 | 8.27E−04 | 6.68E−09 | 100.0 | nm | 52 | 162.98 | 0.04 | 0.39 | 4.00 |
| VH Y30W | 0.00 | 7.23E+06 | 4.66E−03 | 6.44E−09 | 100.0 | nm | 52 | 149.94 | 0.04 | 0.39 | 4.00 |
| VH Y31A | 0.00 | nd | nd | nd | 99.7 | 29.3 | 52 | 150.08 | 0.04 | 0.39 | 4.00 |
| VH Y31D | 0.00 | nd | nd | nd | 99.3 | nm | 52 | 148.50 | 0.04 | 0.45 | 0.00 |
| VH Y31E | 5.17 | nd | nd | nd | 99.7 | nm | 52 | 148.47 | 0.04 | 0.45 | 0.00 |
| VH Y31F | 0.00 | 1.14E+06 | 7.94E−04 | 6.94E−09 | 100.0 | nm | 52 | 161.77 | 0.04 | 0.39 | 4.00 |
| VH Y31H | 15.10 | 3.52E+06 | 6.15E−03 | 1.75E−08 | 99.9 | 26.1 | 52 | 147.62 | 0.05 | 0.39 | 4.40 |
| VH Y31I | 7.97 | nd | nd | nd | 97.6 | nm | 52 | 151.71 | 0.04 | 0.39 | 4.00 |
| VH Y31K | 16.77 | nd | nd | nd | 99.8 | 21.0 | 52 | 135.58 | 0.07 | 0.11 | 16.00 |
| VH Y31L | 7.00 | 9.23E+04 | 2.44E−02 | 2.64E−06 | 100.0 | nm | 52 | 162.02 | 0.04 | 0.39 | 4.00 |
| VH Y31N | 10.80 | nd | nd | nd | 99.8 | nm | 52 | 148.42 | 0.04 | 0.39 | 4.00 |
| VH Y31W | 9.56 | nd | nd | nd | 99.9 | nm | 52 | 158.32 | 0.04 | 0.39 | 4.00 |
| VH Y31R | 15.02 | nd | nd | nd | 99.9 | 27.5 | 52 | 142.36 | 0.07 | 0.19 | 12.00 |
| VH Y31S | 10.44 | nd | nd | nd | 99.9 | nm | 52 | 149.07 | 0.04 | 0.39 | 4.00 |
| VH Y31T | 9.73 | 1.86E+06 | 3.60E−02 | 1.94E−07 | 99.9 | nm | 52 | 149.89 | 0.04 | 0.39 | 4.00 |
| VH Y31V | 3.80 | 1.02E+06 | 4.13E−02 | 4.06E−07 | 100.0 | nm | 52 | 151.64 | 0.04 | 0.39 | 4.00 |
| VH Y33A | 4.01 | 6.05E+05 | 1.62E−02 | 2.67E−07 | 100.0 | 49.9 | 52 | 147.87 | 0.04 | 0.39 | 4.00 |
| VH Y53A | 10.34 | nd | nd | nd | 100.0 | 21.0 | 52 | 149.32 | 0.04 | 0.39 | 4.00 |
| VH Y53D | 8.96 | nd | nd | nd | 99.8 | nm | 52 | 143.05 | 0.04 | 0.44 | 0.00 |
| VH Y53E | 10.52 | nd | nd | nd | 99.9 | nm | 52 | 159.16 | 0.04 | 0.45 | 0.00 |
| VH Y53F | 0.00 | 3.02E+06 | 1.16E−02 | 3.83E−08 | 100.0 | nm | 52 | 144.14 | 0.04 | 0.39 | 4.00 |
| VH Y53H | 14.02 | 4.73E+06 | 1.02E−01 | 2.16E−07 | 100.0 | 24.8 | 52 | 152.82 | 0.04 | 0.39 | 4.40 |
| VH Y53I | 0.00 | nd | nd | nd | 100.0 | nm | 52 | 154.88 | 0.04 | 0.39 | 4.00 |
| VH Y53K | 20.04 | nd | nd | nd | 100.0 | 15.7 | 52 | 158.88 | 0.04 | 0.39 | 8.00 |
| VH Y53L | 0.00 | 9.47E+05 | 3.04E−02 | 3.21E−07 | 100.0 | nm | 52 | 144.55 | 0.04 | 0.39 | 4.00 |
| VH Y53N | 8.88 | nd | nd | nd | 94.3 | nm | 52 | 143.05 | 0.04 | 0.39 | 4.00 |
| VH Y53Q | 13.06 | nd | nd | nd | 98.3 | nm | 52 | 148.99 | 0.04 | 0.39 | 4.00 |
| VH Y53R | 19.90 | nd | nd | nd | 98.7 | 19.1 | 52 | 141.22 | 0.04 | 0.39 | 8.00 |
| VH Y53S | 7.24 | nd | nd | nd | 98.4 | nm | 52 | 149.08 | 0.04 | 0.39 | 4.00 |
| VH Y53T | 12.43 | 2.27E+06 | 5.31E−02 | 2.34E−07 | 97.2 | nm | 52 | 142.60 | 0.04 | 0.39 | 4.00 |
| VH Y53V | 0.00 | 9.97E+05 | 5.02E−02 | 5.04E−07 | 99.9 | nm | 52 | 150.69 | 0.04 | 0.39 | 4.00 |
| VH Y58A | 0.00 | 2.75E+06 | 1.02E−03 | 3.71E−09 | 99.1 | 38.2 | 52 | 155.25 | 0.04 | 0.39 | 4.00 |
| VH Y58D | 0.00 | 3.80E+06 | 1.22E−03 | 3.21E−09 | 98.4 | nm | 52 | 161.89 | 0.04 | 0.39 | 0.00 |
| VH Y58E | 1.12 | 3.12E+06 | 1.20E−03 | 3.83E−09 | 98.3 | nm | 52 | 161.81 | 0.04 | 0.39 | 0.00 |
| VH Y58F | 6.66 | 3.80E+06 | 1.04E−03 | 2.73E−09 | 93.6 | nm | 52 | 165.12 | 0.04 | 0.39 | 4.00 |
| VF Y58H | 9.11 | 2.56E+06 | 1.08E−03 | 4.21E−09 | 98.1 | nm | 52 | 162.11 | 0.04 | 0.39 | 4.40 |
| VH Y58I | 0.00 | 2.91E+06 | 7.89E−04 | 2.72E−09 | 99.2 | nm | 52 | 148.42 | 0.04 | 0.39 | 4.00 |
| VH Y58K | 6.51 | 2.28E+06 | 1.36E−03 | 5.95E−09 | 98.1 | nm | 52 | 156.88 | 0.04 | 0.39 | 8.00 |
| VH Y58L | 0.00 | 3.18E+06 | 7.16E−04 | 2.25E−09 | 98.3 | nm | 52 | 165.92 | 0.04 | 0.39 | 4.00 |
| VH Y58N | 0.00 | 3.20E+06 | 1.22E−03 | 3.83E−09 | 98.3 | nm | 52 | 151.92 | 0.04 | 0.39 | 4.00 |
| VH Y58Q | 5.33 | 2.92E+06 | 1.03E−03 | 3.54E−09 | 98.4 | nm | 52 | 159.28 | 0.04 | 0.39 | 4.00 |
| VH Y58R | 9.04 | 2.89E+06 | 1.67E−03 | 5.77E−09 | 97.5 | nm | 52 | 158.02 | 0.04 | 0.39 | 8.00 |
| VH Y58S | 6.08 | 2.71E+06 | 1.01E−03 | 3.74E−09 | 98.3 | nm | 52 | 163.05 | 0.04 | 0.39 | 4.00 |
| VH Y58T | 7.26 | 2.33E+06 | 8.62E−04 | 3.70E−09 | 92.1 | nm | 52 | 153.13 | 0.04 | 0.39 | 4.00 |
| VH Y58V | 0.00 | 3.23E+06 | 1.02E−03 | 3.15E−09 | 98.3 | nm | 52 | 151.63 | 0.04 | 0.39 | 4.00 |
| VH Y58W | 4.46 | nd | nd | nd | 98.4 | nm | 52 | 163.50 | 0.04 | 0.39 | 4.00 |
| VL S93D | 0.00 | 2.54E+06 | 3.29E−04 | 1.30E−09 | 100.0 | nm | 52 | 148.39 | 0.04 | 0.43 | 3.00 |
| VL S93E | 0.00 | 2.15E+06 | 2.08E−04 | 9.68E−10 | 99.3 | nm | 52 | 158.44 | 0.04 | 0.44 | 3.00 |
| VL S93F | 0.00 | 7.21E+05 | 4.03E−04 | 5.59E−09 | 98.8 | nm | 52 | 150.67 | 0.04 | 0.39 | 4.00 |
| VL S93H | 6.64 | 2.38E+06 | 5.66E−04 | 2.38E−09 | 98.9 | nm | 52 | 148.62 | 0.04 | 0.39 | 4.10 |
| VL S93I | 5.14 | 1.22E+06 | 2.79E−04 | 2.28E−09 | 95.5 | nm | 52 | 152.00 | 0.04 | 0.39 | 4.00 |
| VL S93K | 9.54 | 2.46E+06 | 7.16E−04 | 2.91E−09 | 98.8 | nm | 52 | 148.18 | 0.04 | 0.39 | 5.00 |
| VL S93N | 6.38 | 4.27E+06 | 4.11E−04 | 9.62E−10 | 98.5 | nm | 52 | 158.58 | 0.04 | 0.39 | 4.00 |
| VL S93Q | 5.66 | 2.79E+06 | 6.08E−04 | 2.18E−09 | 98.4 | nm | 52 | 158.35 | 0.04 | 0.39 | 4.00 |
| VL S93R | 11.29 | 1.90E+06 | 1.45E−04 | 7.66E−10 | 98.8 | nm | 52 | 148.08 | 0.04 | 0.39 | 5.00 |
| VL S93T | 5.38 | 2.01E+06 | 2.57E−04 | 1.27E−09 | 98.4 | nm | 52 | 159.67 | 0.04 | 0.39 | 4.00 |
| VL S93V | 0.00 | 1.56E+06 | 2.72E−04 | 1.74E−09 | 94.1 | nm | 52 | 150.96 | 0.04 | 0.39 | 4.00 |
| VL Y91A | 6.06 | 2.57E+06 | 2.53E−02 | 9.85E−08 | 96.1 | nm | 52 | 167.58 | 0.04 | 0.39 | 4.00 |
| VL Y91D | 0.00 | 1.07E+06 | 7.55E−04 | 7.08E−09 | 98.9 | nm | 52 | 180.88 | 0.04 | 0.39 | 4.00 |
| VL Y91E | 0.00 | 2.09E+06 | 7.14E−04 | 3.42E−09 | 97.7 | nm | 52 | 167.58 | 0.04 | 0.39 | 4.00 |
| VL Y91F | 0.00 | 3.93E+06 | 6.66E−04 | 1.69E−09 | 96.5 | nm | 52 | 148.72 | 0.04 | 0.39 | 4.00 |
| VL Y91H | 8.56 | 2.61E+06 | 7.38E−04 | 2.82E−09 | 95.6 | nm | 52 | 153.58 | 0.05 | 0.39 | 4.10 |
| VL Y91K | 7.10 | 2.23E+06 | 4.64E−04 | 2.08E−09 | 91.0 | nm | 52 | 174.43 | 0.04 | 0.39 | 4.00 |
| VL Y91L | 0.00 | 1.92E+06 | 8.05E−04 | 4.20E−09 | 95.6 | nm | 52 | 167.58 | 0.04 | 0.39 | 4.00 |
| VL Y91Q | 0.00 | 2.16E+06 | 5.11E−04 | 2.36E−09 | 97.2 | nm | 52 | 180.88 | 0.04 | 0.39 | 4.00 |
| VL Y91S | 9.89 | 2.09E+06 | 4.84E−04 | 2.31E−09 | 97.1 | nm | 52 | 167.30 | 0.04 | 0.39 | 4.00 |
| VL Y91T | 4.83 | 1.82E+06 | 4.42E−04 | 2.42E−09 | 97.6 | nm | 52 | 167.30 | 0.04 | 0.39 | 4.00 |
| VL Y91W | 4.87 | 3.80E+06 | 1.15E−03 | 3.02E−09 | 94.9 | nm | 52 | 162.86 | 0.04 | 0.39 | 4.00 |
| VL Y94A | 6.47 | 1.90E+06 | 5.01E−04 | 2.64E−09 | 97.1 | 39.1 | 52 | 169.39 | 0.04 | 0.29 | 4.00 |
| VL Y94D | 0.00 | 2.15E+06 | 4.12E−04 | 1.92E−09 | 99.4 | nm | 52 | 168.36 | 0.04 | 0.47 | 3.00 |
| VL Y94E | 0.00 | 2.18E+06 | 4.02E−04 | 1.85E−09 | 99.4 | nm | 52 | 160.57 | 0.04 | 0.29 | 3.00 |
| VL Y94F | 5.92 | 2.00E+06 | 4.47E−04 | 2.23E−09 | 98.1 | nm | 52 | 161.18 | 0.04 | 0.39 | 4.00 |
| VL Y94H | 7.54 | 2.19E+06 | 6.62E−04 | 3.02E−09 | 94.2 | nm | 52 | 165.52 | 0.04 | 0.39 | 4.10 |
| VL Y94K | 15.14 | 3.31E+06 | 5.31E−04 | 1.60E−09 | 99.1 | 17.1 | 52 | 160.88 | 0.04 | 0.29 | 5.00 |
| VL Y94L | 9.54 | 1.71E+06 | 3.52E−04 | 2.05E−09 | 99.4 | nm | 52 | 175.92 | 0.04 | 0.39 | 4.00 |
| VL Y94N | 8.21 | 2.78E+06 | 5.11E−04 | 1.84E−09 | 98.5 | nm | 52 | 172.29 | 0.04 | 0.39 | 4.00 |
| VL Y94Q | 7.88 | 2.86E+06 | 5.93E−04 | 2.08E−09 | 97.0 | nm | 52 | 167.49 | 0.04 | 0.29 | 4.00 |
| VL Y94R | 11.98 | 2.21E+06 | 3.11E−04 | 1.41E−09 | 98.7 | nm | 52 | 171.67 | 0.04 | 0.39 | 5.00 |
| VL Y94S | 8.45 | 2.03E+06 | 4.21E−04 | 2.08E−09 | 98.5 | nm | 52 | 168.24 | 0.04 | 0.29 | 4.00 |
| VL Y94T | 8.75 | 1.68E+06 | 4.56E−04 | 2.71E−09 | 97.8 | nm | 52 | 163.21 | 0.04 | 0.29 | 4.00 |
| VL Y94V | 7.04 | 1.59E+06 | 3.92E−04 | 2.46E−09 | 99.2 | nm | 52 | 171.25 | 0.04 | 0.29 | 4.00 |
| VL Y94W | 10.98 | 1.85E+06 | 4.72E−04 | 2.55E−09 | 100.0 | nm | 52 | 158.61 | 0.04 | 0.39 | 4.00 |

TABLE 3B
Multi-parameter evaluation of parent and variant antibodies.

| | | | | | | Isoelectric | | |
|---|---|---|---|---|---|---|---|---|
| Anti-GCGR | TAP flags | | | | | point (pI) | | |
| IgG₁ | (Green, Amber, Red) | | | | | IgG₁ | | SAP |
| antibody | CDRL | PSH | PPC | PNC | SFvCSP | (minus HC C- | | score |
| variants | flag | flag | flag | flag | flag | terminal Lys) | Fv | Fv |
| Parent | Green | Green | Green | Green | Green | 8.02 | 7.75 | 73.38 |
| VH S54A | Green | Green | Green | Green | Green | 8.02 | 7.75 | 72.38 |
| VH S54D | Green | Green | Green | Green | Green | 7.82 | 6.77 | 63.10 |
| VH S54H | Green | Green | Green | Green | Green | 8.02 | 7.77 | 60.99 |
| VH S54K | Green | Green | Green | Green | Green | 8.17 | 8.26 | 63.24 |
| VH S54L | Green | Green | Green | Green | Green | 8.02 | 7.75 | 78.02 |
| VH S54N | Green | Green | Green | Green | Green | 8.02 | 7.75 | 66.22 |
| VH S54R | Green | Green | Green | Green | Green | 8.17 | 8.27 | 67.23 |
| VH S54T | Green | Green | Green | Green | Green | 8.02 | 7.75 | 68.97 |
| VH S54V | Green | Green | Green | Green | Green | 8.02 | 7.75 | 72.71 |
| VH S56A | Green | Green | Green | Green | Green | 8.02 | 7.75 | 70.51 |
| VH S56F | Green | Green | Green | Green | Green | 8.02 | 7.75 | 88.67 |
| VH S56L | Green | Green | Green | Green | Green | 8.02 | 7.75 | 75.05 |
| VH S56N | Green | Green | Green | Green | Green | 8.02 | 7.75 | 69.18 |
| VH S56Q | Green | Green | Green | Green | Green | 8.02 | 7.75 | 67.66 |
| VH S56T | Green | Green | Green | Green | Green | 8.02 | 7.75 | 68.48 |
| VH S56V | Green | Green | Green | Green | Green | 8.02 | 7.75 | 72.59 |
| VH V99A | Green | Green | Green | Green | Green | 8.02 | 7.75 | 67.82 |
| VH V99D | Green | Green | Green | Green | Green | 7.82 | 6.77 | 95.62 |
| VH V99E | Green | Green | Green | Green | Green | 7.82 | 6.77 | 89.05 |
| VH V99H | Green | Green | Green | Green | Green | 8.02 | 7.77 | 97.89 |
| VH V99I | Green | Amber | Green | Green | Green | 8.02 | 7.75 | 73.55 |
| VH V99K | Green | Green | Green | Green | Green | 8.17 | 8.26 | 97.84 |
| VH V99L | Green | Green | Green | Green | Green | 8.02 | 7.75 | 76.65 |
| VH V99N | Green | Green | Green | Green | Green | 8.02 | 7.75 | 102.56 |
| VH V99Q | Green | Green | Green | Green | Green | 8.02 | 7.75 | 101.90 |
| VH V99R | Green | Green | Green | Green | Green | 8.17 | 8.27 | 99.91 |
| VH V99S | Green | Green | Green | Green | Green | 8.02 | 7.75 | 105.46 |
| VH V99T | Green | Green | Green | Green | Green | 8.02 | 7.75 | 66.84 |
| VH W100aA | Green | Amber | Green | Green | Green | 8.02 | 7.75 | 74.87 |
| VH W100aD | Green | Green | Green | Green | Green | 7.82 | 6.77 | 68.64 |
| VH W100aE | Green | Green | Green | Green | Green | 7.82 | 6.77 | 71.15 |
| VH W100aF | Green | Green | Green | Green | Green | 8.02 | 7.75 | 92.45 |
| VH W100aH | Green | Green | Green | Green | Green | 8.02 | 7.77 | 70.08 |
| VH W100aI | Green | Green | Green | Green | Green | 8.02 | 7.75 | 85.48 |
| VH W100aK | Green | Green | Green | Green | Green | 8.17 | 8.26 | 72.51 |
| VH W100aL | Green | Amber | Green | Green | Green | 8.02 | 7.75 | 85.02 |
| VH W100aN | Green | Green | Green | Green | Green | 8.02 | 7.75 | 76.57 |
| VH W100aQ | Green | Green | Green | Green | Green | 8.02 | 7.75 | 74.62 |
| VH W100aR | Green | Green | Green | Green | Green | 8.17 | 8.27 | 74.09 |
| VH W100aS | Green | Green | Green | Green | Green | 8.02 | 7.75 | 104.80 |
| VH W100aT | Green | Green | Green | Green | Green | 8.02 | 7.75 | 78.34 |
| VH W100aV | Green | Green | Green | Green | Green | 8.02 | 7.75 | 79.93 |
| VH W100aY | Green | Green | Green | Green | Green | 8.02 | 7.75 | 92.92 |
| VH W100bA | Green | Green | Green | Green | Green | 8.02 | 7.76 | 70.65 |
| VH W100bD | Green | Green | Green | Green | Green | 7.82 | 6.77 | 66.67 |
| VH W100bE | Green | Green | Green | Green | Green | 7.82 | 6.77 | 64.96 |
| VH W100bF | Green | Green | Green | Green | Green | 8.02 | 7.76 | 68.50 |
| VH W100bH | Green | Green | Green | Green | Green | 8.02 | 7.77 | 63.76 |
| VH W100bI | Green | Green | Green | Green | Green | 8.02 | 7.76 | 68.01 |
| VH Y100bK | Green | Green | Green | Green | Green | 8.17 | 8.27 | 63.83 |
| VH Y100bL | Green | Green | Green | Green | Green | 8.02 | 7.76 | 67.38 |
| VH Y100bN | Green | Green | Green | Green | Green | 8.02 | 7.76 | 67.87 |
| VH Y100bQ | Green | Green | Green | Green | Green | 8.02 | 7.76 | 65.20 |
| VH Y100bR | Green | Green | Green | Green | Green | 8.18 | 8.27 | 69.54 |
| VH Y100bS | Green | Green | Green | Green | Green | 8.02 | 7.76 | 68.59 |
| VH Y100bT | Green | Green | Green | Green | Green | 8.02 | 7.76 | 71.53 |
| VH Y100bV | Green | Green | Green | Green | Green | 8.02 | 7.76 | 67.41 |
| VH Y100dA | Green | Green | Green | Green | Green | 8.02 | 7.76 | 97.77 |
| VH Y100dD | Green | Green | Green | Green | Green | 7.82 | 6.77 | 91.30 |
| VH Y100dE | Green | Green | Green | Green | Green | 7.82 | 6.77 | 90.56 |
| VH Y100dF | Green | Amber | Green | Green | Green | 8.02 | 7.76 | 75.56 |
| VH Y100dI | Green | Green | Green | Green | Green | 8.02 | 7.76 | 97.02 |
| VH Y100dK | Green | Green | Green | Green | Green | 8.17 | 8.27 | 97.07 |
| VH Y100dN | Green | Green | Green | Green | Green | 8.02 | 7.76 | 97.74 |
| VH Y100dQ | Green | Green | Green | Green | Green | 8.02 | 7.76 | 97.73 |
| VH Y100dR | Green | Green | Green | Green | Green | 8.18 | 8.27 | 98.23 |
| VH Y100dS | Green | Green | Green | Green | Green | 8.02 | 7.76 | 97.02 |
| VH Y100dT | Green | Green | Green | Green | Green | 8.02 | 7.76 | 95.25 |
| VH Y30A | Green | Green | Green | Green | Green | 8.02 | 7.76 | 61.85 |
| VH Y30D | Green | Green | Green | Green | Green | 7.82 | 6.77 | 50.72 |
| VH Y30E | Green | Green | Green | Green | Green | 7.82 | 6.77 | 44.68 |
| VH Y30F | Green | Green | Green | Green | Green | 8.02 | 7.76 | 71.45 |
| VH Y30H | Green | Green | Green | Green | Green | 8.02 | 7.77 | 52.61 |
| VH Y30I | Green | Green | Green | Green | Green | 8.02 | 7.76 | 62.70 |
| VH Y30K | Green | Green | Green | Green | Green | 8.17 | 8.27 | 51.75 |
| VH Y30L | Green | Green | Green | Green | Green | 8.02 | 7.76 | 67.18 |
| VH Y30N | Green | Green | Green | Green | Green | 8.02 | 7.76 | 54.99 |
| VH Y30Q | Green | Green | Green | Green | Green | 8.02 | 7.76 | 55.03 |
| VH Y30R | Green | Green | Green | Green | Green | 8.18 | 8.27 | 56.25 |
| VH Y30S | Green | Green | Green | Green | Green | 8.02 | 7.76 | 60.34 |
| VH Y30T | Green | Green | Green | Green | Green | 8.02 | 7.76 | 58.90 |
| VH Y30V | Green | Green | Green | Green | Green | 8.02 | 7.76 | 62.08 |
| VH Y30W | Green | Green | Green | Green | Green | 8.02 | 7.76 | 81.87 |
| VH Y31A | Green | Green | Green | Green | Green | 8.02 | 7.76 | 63.07 |
| VH Y31D | Green | Green | Green | Green | Green | 7.82 | 6.77 | 50.42 |
| VH Y31E | Green | Green | Green | Green | Green | 7.82 | 6.77 | 53.11 |
| VH Y31F | Green | Green | Green | Green | Green | 8.02 | 7.76 | 77.16 |
| VH Y31H | Green | Green | Green | Green | Green | 8.02 | 7.77 | 49.98 |
| VH Y31I | Green | Green | Green | Green | Green | 8.02 | 7.76 | 71.23 |
| VH Y31K | Green | Green | Green | Green | Green | 8.17 | 8.27 | 60.79 |
| VH Y31L | Green | Green | Green | Green | Green | 8.02 | 7.76 | 71.48 |
| VH Y31N | Green | Green | Green | Green | Green | 8.02 | 7.76 | 59.24 |
| VH Y31Q | Green | Green | Green | Green | Green | 8.02 | 7.76 | 57.66 |
| VH Y31R | Green | Green | Green | Green | Green | 8.18 | 8.27 | 59.96 |
| VH Y31S | Green | Green | Green | Green | Green | 8.02 | 7.76 | 63.42 |
| VH Y31T | Green | Green | Green | Green | Green | 8.02 | 7.76 | 67.44 |
| VH Y31V | Green | Green | Green | Green | Green | 8.02 | 7.76 | 69.76 |
| VH Y33A | Green | Green | Green | Green | Green | 8.02 | 7.76 | 75.25 |
| VH Y53A | Green | Green | Green | Green | Green | 8.02 | 7.76 | 60.13 |
| VH Y53D | Green | Green | Green | Green | Green | 7.82 | 6.77 | 49.82 |
| VH Y53E | Green | Green | Green | Green | Green | 7.82 | 6.77 | 47.55 |
| VH Y53F | Green | Green | Green | Green | Green | 8.02 | 7.76 | 77.50 |
| VH Y53H | Green | Green | Green | Green | Green | 8.02 | 7.77 | 51.66 |
| VH Y53I | Green | Green | Green | Green | Green | 8.02 | 7.76 | 66.87 |
| VH Y53K | Green | Green | Green | Green | Green | 8.17 | 8.27 | 55.57 |
| VH Y53L | Green | Green | Green | Green | Green | 8.02 | 7.76 | 70.27 |
| VH Y53N | Green | Green | Green | Green | Green | 8.02 | 7.76 | 57.90 |
| VH Y53Q | Green | Green | Green | Green | Green | 8.02 | 7.76 | 60.42 |
| VH Y53R | Green | Green | Green | Green | Green | 8.18 | 8.27 | 51.10 |
| VH Y53S | Green | Green | Green | Green | Green | 8.02 | 7.76 | 58.39 |
| VH Y53T | Green | Green | Green | Green | Green | 8.02 | 7.76 | 55.67 |
| VH Y53V | Green | Green | Green | Green | Green | 8.02 | 7.76 | 61.28 |
| VH Y58A | Green | Green | Green | Green | Green | 8.02 | 7.76 | 71.45 |
| VH Y58D | Green | Green | Green | Green | Green | 7.82 | 6.77 | 71.82 |
| VH Y58E | Green | Green | Green | Green | Green | 7.82 | 6.77 | 67.38 |
| VH Y58F | Green | Green | Green | Green | Green | 8.02 | 7.76 | 74.40 |
| VH Y58H | Green | Green | Green | Green | Green | 8.02 | 7.77 | 64.60 |
| VH Y58I | Green | Green | Green | Green | Green | 8.02 | 7.76 | 74.50 |
| VH Y58K | Green | Green | Green | Green | Green | 8.17 | 8.27 | 66.39 |
| VH Y58L | Green | Green | Green | Green | Green | 8.02 | 7.76 | 78.12 |
| VH Y58N | Green | Green | Green | Green | Green | 8.02 | 7.76 | 70.89 |
| VH Y58Q | Green | Green | Green | Green | Green | 8.02 | 7.76 | 72.29 |
| VH Y58R | Green | Green | Green | Green | Green | 8.18 | 8.27 | 64.83 |
| VH Y58S | Green | Green | Green | Green | Green | 8.02 | 7.76 | 68.74 |
| VH Y58T | Green | Green | Green | Green | Green | 8.02 | 7.76 | 72.69 |
| VH Y58V | Green | Green | Green | Green | Green | 8.02 | 7.76 | 73.59 |
| VH Y58W | Green | Green | Green | Green | Green | 8.02 | 7.76 | 70.85 |
| VL S93D | Green | Green | Green | Green | Green | 7.82 | 6.77 | 70.82 |
| VL S93E | Green | Green | Green | Green | Green | 7.82 | 6.77 | 63.37 |
| VL S93F | Green | Green | Green | Green | Green | 8.02 | 7.75 | 81.57 |
| VL S93H | Green | Green | Green | Green | Green | 8.02 | 7.77 | 74.30 |
| VL S93I | Green | Green | Green | Green | Green | 8.02 | 7.75 | 71.50 |
| VL S93K | Green | Green | Green | Green | Green | 8.17 | 8.26 | 63.13 |
| VL S93N | Green | Green | Green | Green | Green | 8.02 | 7.75 | 69.36 |
| VL S93Q | Green | Green | Green | Green | Green | 8.02 | 7.75 | 64.80 |
| VL S93R | Green | Green | Green | Green | Green | 8.17 | 8.27 | 64.23 |
| VL S93T | Green | Green | Green | Green | Green | 8.02 | 7.75 | 72.80 |
| VL S93V | Green | Green | Green | Green | Green | 8.02 | 7.75 | 70.51 |
| VL Y91A | Green | Green | Green | Green | Green | 8.02 | 7.76 | 71.06 |
| VL Y91D | Green | Amber | Green | Green | Green | 7.82 | 6.77 | 70.89 |
| VL Y91E | Green | Green | Green | Green | Green | 7.82 | 6.77 | 69.63 |
| VL Y91F | Green | Green | Green | Green | Green | 8.02 | 7.76 | 69.55 |
| VL Y91H | Green | Green | Green | Green | Green | 8.02 | 7.77 | 72.79 |
| VL Y91K | Green | Amber | Green | Green | Green | 8.17 | 8.27 | 69.34 |
| VL Y91L | Green | Green | Green | Green | Green | 8.02 | 7.76 | 72.15 |
| VL Y91Q | Green | Amber | Green | Green | Green | 8.02 | 7.76 | 67.03 |
| VL Y91S | Green | Green | Green | Green | Green | 8.02 | 7.76 | 70.36 |
| VL Y91T | Green | Green | Green | Green | Green | 8.02 | 7.76 | 71.59 |
| VL Y91W | Green | Green | Green | Green | Green | 8.02 | 7.76 | 65.63 |
| VL Y94A | Green | Green | Green | Green | Green | 8.02 | 7.76 | 66.26 |
| VL Y94D | Green | Green | Green | Green | Green | 7.82 | 6.77 | 54.12 |
| VL Y94E | Green | Green | Green | Green | Green | 7.82 | 6.77 | 60.53 |
| VL Y94F | Green | Green | Green | Green | Green | 8.02 | 7.76 | 79.73 |
| VL Y94H | Green | Green | Green | Green | Green | 8.02 | 7.77 | 67.49 |
| VL Y94K | Green | Green | Green | Green | Green | 8.17 | 8.27 | 64.19 |
| VL Y94L | Green | Amber | Green | Green | Green | 8.02 | 7.76 | 64.34 |
| VL Y94N | Green | Green | Green | Green | Green | 8.02 | 7.76 | 65.65 |
| VL Y94Q | Green | Green | Green | Green | Green | 8.02 | 7.76 | 66.42 |
| VL Y94R | Green | Green | Green | Green | Green | 8.18 | 8.27 | 62.81 |
| VL Y94S | Green | Green | Green | Green | Green | 8.02 | 7.76 | 70.29 |
| VL Y94T | Green | Green | Green | Green | Green | 8.02 | 7.76 | 70.96 |
| VL Y94V | Green | Green | Green | Green | Green | 8.02 | 7.76 | 66.52 |
| VL Y94W | Green | Green | Green | Green | Green | 8.02 | 7.76 | 70.04 |

The amino acid sequences of the VH domains of the Y100bV, W100aF, S56R, Y100bA, Y100bH, Y30H, W100aV, W100aL, Y100bK. W100aA, S54R, V99K, Y31H are shown below:

(SEQ ID NO: 3)
EVQLVESGGGLVQPGGSLRLSCAASGFNIYYNYIHWVRQAPGKGLEWVAEFSPYSGSTYYA
DSVKGRFTISADTSKNTAYLQMNSLRAEDTAVYYCARSAAIKDWYDYFKGFDYWGQGTLV
TVSS
V99K
(SEQ ID NO: 4)
EVQLVESGGGLVQPGGSLRLSCAASGFNIYYNYIHWVRQAPGKGLEWVAEFSPYSGSTYYA
DSVKGRFTISADTSKNTAYLQMNSLRAEDTAVYYCARSAAIVDFYDYFKGFDYWGQGTLV
TVSS
W100aF
(SEQ ID NO: 5)
EVQLVESGGGLVQPGGSLRLSCAASGFNIYYNYIHWVRQAPGKGLEWVAEFSPYSGSTYYA
DSVKGRFTISADTSKNTAYLQMNSLRAEDTAVYYCARSAAIVDVYDYFKGFDYWGQGTLV
TVSS
W100aV
(SEQ ID NO: 6)
EVQLVESGGGLVQPGGSLRLSCAASGFNIYYNYIHWVRQAPGKGLEWVAEFSPYSGSTYYA
DSVKGRFTISADTSKNTAYLQMNSLRAEDTAVYYCARSAAIVDLYDYFKGFDYWGQGTLV
TVSS
W100aL
(SEQ ID NO: 7)
EVQLVESGGGLVQPGGSLRLSCAASGFNIYYNYIHWVRQAPGKGLEWVAEFSPYSGSTYYA
DSVKGRFTISADTSKNTAYLQMNSLRAEDTAVYYCARSAAIVDAYDYFKGFDYWGQGTLV
TVSS
W100aA
(SEQ ID NO: 8)
EVQLVESGGGLVQPGGSLRLSCAASGFNIYYNYIHWVRQAPGKGLEWVAEFSPYSGSTYYA
DSVKGRFTISADTSKNTAYLQMNSLRAEDTAVYYCARSAAIVDWVDYFKGFDYWGQGTLV
TVSS
Y100bV
(SEQ ID NO: 9)
EVQLVESGGGLVQPGGSLRLSCAASGFNIYYNYIHWVRQAPGKGLEWVAEFSPYSGSTYYA
DSVKGRFTISADTSKNTAYLQMNSLRAEDTAVYYCARSAAIVDWADYFKGFDYWGQGTLV
TVSS
Y100bA
(SEQ ID NO: 10)
EVQLVESGGGLVQPGGSLRLSCAASGFNIYYNYIHWVRQAPGKGLEWVAEFSPYSGSTYYA
DSVKGRFTISADTSKNTAYLQMNSLRAEDTAVYYCARSAAIVDWHDYFKGFDYWGQGTLV
TVSS
Y100bH
(SEQ ID NO: 11)
EVQLVESGGGLVQPGGSLRLSCAASGFNIYYNYIHWVRQAPGKGLEWVAEFSPYSGSTYYA
DSVKGRFTISADTSKNTAYLQMNSLRAEDTAVYYCARSAAIVDWKDYFKGFDYWGQGTLV
TVSS
Y100bK
(SEQ ID NO: 12)
EVQLVESGGGLVQPGGSLRLSCAASGFNIHYNYIHWVRQAPGKGLEWVAEFSPYSGSTYYA
DSVKGRFTISADTSKNTAYLQMNSLRAEDTAVYYCARSAAIVDWYDYFKGFDYWGQGTLV
TVSS
Y30H
(SEQ ID NO: 13)
EVQLVESGGGLVQPGGSLRLSCAASGFNIYHNYIHWVRQAPGKGLEWVAEFSPYSGSTYYA
DSVKGRFTISADTSKNTAYLQMNSLRAEDTAVYYCARSAAIVDWYDYFKGFDYWGQGTLV
TVSS
Y31H
(SEQ ID NO: 14)
EVQLVESGGGLVQPGGSLRLSCAASGFNIYYNYIHWVRQAPGKGLEWVAEFSPYRGSTYYA
DSVKGRFTISADTSKNTAYLQMNSLRAEDTAVYYCARSAAIVDWYDYFKGFDYWGQGTLV
TVSS
S54R
(SEQ ID NO: 15)
EVQLVESGGGLVQPGGSLRLSCAASGFNIYYNYIHWVRQAPGKGLEWVAEFSPYSGRTYYA
DSVKGRFTISADTSKNTAYLQMNSLRAEDTAVYYCARSAAIVDWYDYFKGFDYWGQGTLV
TVSS
S56R

Additional tested amino acid sequences are also included in this application based on the information provided herein, e.g., FIG. 9, including:

(SEQ ID NO: 19)
EVQLVESGGGLVQPGGSLRLSCAASGFNIYYNYIHWVRQAPGKGLEWVAEFSPYSGST
YYADSVKGRFTISADTSKNTAYLQMNSLRAEDTAVYYCARSAAIVDHYDYFKGFDYW
GQGTLVTVSS
W100aH
(SEQ ID NO: 20)
EVQLVESGGGLVQPGGSLRLSCAASGFNIYYNYIHWVRQAPGKGLEWVAEFSPYSGST
YYADSVKGRFTISADTSKNTAYLQMNSLRAEDTAVYYCARSAAIVDKYDYFKGFDYW
GQGTLVTVSS
W100aK
(SEQ ID NO: 21)
EVQLVESGGGLVQPGGSLRLSCAASGFNIYYNYIHWVRQAPGKGLEWVAEFSPYSGST
YYADSVKGRFTISADTSKNTAYLQMNSLRAEDTAVYYCARSAAIVDRYDYFKGFDYW
GQGTLVTVSS
W100aR
(SEQ ID NO: 22)
EVQLVESGGGLVQPGGSLRLSCAASGFNIYYNYIHWVRQAPGKGLEWVAEFSPYSGST
YYADSVKGRFTISADTSKNTAYLQMNSLRAEDTAVYYCARSAAIVDWHDYFKGFDYW
GQGTLVTVSS
Y100bH

### Summary of Example 1A

The structure of an anti-GCGR Fab comprising the VH set forth in SEQ ID NO:1 and the VL domain set forth in SEQ ID NO:2 was solved to 2.7 Å resolution

Extensive mutational analysis of the VH and VL domains was performed to probe the contribution of hydrophobic patches to self-interaction. Applicants found that key viscosity-affecting residues are characterized by SAP value ≥2 and SASA ratios ≥0.5.

Applicant found that substituting residues having a SAP value ≥2 and a SASA ratio ≥0.5 with a charged or polar residue increases the DLS interaction parameter of the resulting variant. Such variants potentially demonstrate reduced viscosity.

Applicant also identified mutations in SEQ ID NO:1 that both reduce viscosity and maintain antigen-binding affinity.

### Example 1B: Determining the Viscosity of Anti-Glucagon Receptor (GCGR) Antibody Variants Found to Exhibit Increased Interaction Parameter (k_{D}) Relative to the Parental Anti-GCGR Antibody

The viscosities of anti-GCGR IgG1 antibody variants discussed in Example 1A that were found to exhibit increased interaction parameter (k_{D}) relative to the parental anti-GCGR IgG1 antibody are analyzed via rheometry, e.g., as described in Lang et al. (2020) Appl Sci, 10(1), 172 and Zhang et al. (2017) Curr Opin in Chem Eng, 16: 48-55; and Sharma et al. (2011) Soft Matter 10.1039/c0sm01312a, at concentrations of ≤180 mg/ml.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

1. A method of identifying an antigen binding polypeptide variant having reduced viscosity relative to a parental antigen binding polypeptide, comprising:
(a) identifying one or more wild type amino acids in the parental antigen binding polypeptide that have high spatial aggregation propensity (SAP) values and/or high solvent-accessible surface area (SASA) ratios;
(b) substituting the one or more wild type amino acids identified as having the high SAP values and/or the high SASA ratios with amino acids having lower SAP values and/or lower SASA ratios as compared to the wild type amino acids;
(c) measuring the viscosity of the antigen binding polypeptide variant; and
(d) identifying the antigen binding polypeptide variant having reduced viscosity relative to parental polypeptide.

2. The method of claim 1, wherein the one or more wild type amino acids in the parental antigen binding polypeptide are surface exposed.

3. The method of claim 1 or 2, wherein the one or more wild type amino acid(s) are aromatic amino acids, and wherein the one or more aromatic amino acids are substituted with charged amino acids or amino acids having smaller side chains.

4. The method of claim 3, wherein one or more aromatic amino acids are selected from the group consisting of: W, Y, and F.

5. The method of claim 3 or 4, wherein the charged amino acid(s) or amino acid(s) having smaller side chains are selected from the group consisting of A, I, L, V, D, E, H, K, R, N, Q, S, and T.

6. The method of any one of claims 1-5, wherein the one or more wild type amino acid(s) have smaller side chains, and wherein the one or more amino acids that have smaller chains are substituted with charged amino acids.

7. The method of claim 6, wherein the one or more wild type amino acid(s) have smaller side chains are selected from the group consisting of: A, I, L, V, N, Q, S, G, P, C, M, or T.

8. The method of claim 6 or 7, wherein the charged amino acid(s) are selected from the group consisting of: R, K, H, D, and E.

9. The method of any one of claims 1-8, wherein the high SAP values are 2 or greater.

10. The method of claim 9, wherein the high SAP values are 2.3 or greater.

11. The method of any one of claims 1-10, wherein the high SASA ratios are 0.25 or greater.

12. The method of any one of claims 1-11, wherein the high SASA ratios are 0.5 or greater.

13. The method of any one of claims 1-12, wherein the viscosity of the antigen binding polypeptide variant is assessed via dynamic light scattering (DLS).

14. The method of any one of claims 1-13, wherein the viscosity of the antigen binding polypeptide variant is measured via cone and plate rheometry.

15. The method of claim 14, wherein the viscosity of the antigen binding polypeptide variant is measured at a high concentration.

16. The method of claim 15, wherein the high concentration is between about 50 mg/ml and 300 mg/ml.

17. The method of any of claims 1-16,
wherein the parental antigen binding polypeptide is an antibody, an antigen-binding fragment of an antibody, or an antibody construct,
wherein the antibody or antibody construct comprises one or more of a CDR-H1, a CDR-H2, a CDR-H3, a CDR-L1, a CDR-L2 and a CDR-L3, and
wherein one or more wild type amino acids in the one or more CDR-H1, CDR-H2, CDR-H3, CDR-L1, CDR-L2 and CDR-L3 are identified as having high SAP values and/or high SASA ratios.

18. The method of claim 17, wherein the parental antigen binding polypeptide is an antibody.

19. The method of claim 17 or 18, wherein the antibody is a therapeutic antibody.

20. The method of any one of claims 17-19, wherein the antibody is a chimeric antibody, a humanized antibody, or a human antibody.

21. The method of any one of claims 17-20, wherein the antibody is a monospecific antibody, a bispecific antibody, or a multispecific antibody.

22. The method of any one of claims 17-21 wherein the antibody comprises a human IgG Fc region.

23. The method of claim 22, wherein the human IgG constant region is an IgG1, IgG2, or IgG4 Fc region.

24. The method of claim 17, wherein the parental polypeptide is an antigen binding fragment of an antibody.

25. The method of claim 24, wherein the antigen binding fragment is a Fab, a F(ab′)₂, a trispecific Fab₃, an scFv, a monovalent IgG, a diabody, a triabody, an scFv-Vc, a minibody, a V_{H}H, a V-NAR, an hcIgG, or an IgNAR.

26. The method of claim 17, wherein the parental polypeptide is an antibody construct.

27. The method of claim 26, wherein the antibody construct is a CrossMab, a dual action Fab (DAF), a DVD-IgG, or a knob-in-hole bispecific antibody.

28. The method of any one of claims 1-27, further comprising the step of subjecting the antigen binding polypeptide variant to at least one affinity maturation step

29. The method of claim 28, wherein the substituted amino acids in the antigen binding polypeptide variant are not randomized during affinity maturation.

30. A polypeptide variant produced by the method of any one of claims 1-29.

31. A library comprising a plurality of antigen binding polypeptide variants,
wherein at least one variant comprises one or more amino acid substitutions relative to a parental antigen binding polypeptide,
wherein the one or more amino acid substitutions relative to the parental antigen binding polypeptide replace one or more wild type amino acids identified as having the high SAP values and the high SASA ratios with amino acids having lower SAP values and/or lower SASA ratios as compared to the wild type amino acids.

32. The library of claim 31, wherein the plurality comprises at least 1,000 unique variants.

33. The library of claim 31 or 32, wherein the plurality of antigen binding variants is a plurality of antibodies, antigen-binding fragments of antibodies, or antibody constructs.

34. A method of predicting one or more amino acids in an antigen binding polypeptide that affect the viscosity of the antigen binding polypeptide, the method comprising:
identifying one or more amino acids in the antigen binding polypeptide that have high spatial aggregation propensity (SAP) values and/or high solvent-accessible surface area (SASA) ratios,
wherein the one or more amino acids that have high spatial aggregation propensity (SAP) values and/or high solvent-accessible surface area (SASA) ratios are predicted to affect viscosity of the antigen binding polypeptide.

35. A method of predicting one or more amino acids in an antibody, an antigen binding fragment of an antibody, or an antibody construct that affect the viscosity of the antibody, the antigen binding fragment, or the antibody construct, wherein the antibody, the antigen binding fragment, or the antibody construct comprises one or more of a CDR-H1, a CDR-H2, a CDR-H3, a CDR-L1, a CDR-L2 and a CDR-L3, the method comprising:
identifying one or more amino acid positions in the one or more of the CDR-H1, the CDR-H2, the CDR-H3, the CDR-L1, the CDR-L2 and the CDR-L3 that have high spatial aggregation propensity (SAP) values and high solvent-accessible surface area (SASA) ratios,
wherein the one or more amino acid positions that have the high spatial aggregation propensity (SAP) values and the high solvent-accessible surface area (SASA) ratios are predicted to affect viscosity of the antibody, the antigen binding fragment, or the antibody construct.

36. An antibody heavy chain variable domain (V_{H}) set forth in any one of SEQ ID NOs:3-17 or 19-22.

37. An antibody comprising a heavy chain variable domain (V_{H}) set forth in any one of SEQ ID NOs:3-17 or 19-22 and a light chain variable domain (V_{L}) set forth in SEQ ID NO:2 or 18.

38. An antibody comprising a light chain variable domain (V_{L}) set forth in SEQ ID NO: 18.

39. An antibody comprising a light chain variable domain (V_{L}) set forth in SEQ ID NO: 18 and a heavy chain variable domain (V_{H}) set forth in any one of SEQ ID NOs:1, 3-17, or 19-22.
